(19) United States
(12) Patent Application Publication
(10) Pub. No.: US 2025/0034331 A1
(43) Pub. Date: Jan. 30, 2025

(54) POLYESTER URETHANE BIORESORBABLE ELASTOMERS WITH MICROSTRUCTURES FOR TUNABLE DRUG RELEASE AND TUNABLE DEGRADATION

(71) Applicants: SOLESIS, INC., Telford, PA (US); COMPAGNIE GENERALE DES ETABLISSEMENTS MICHELIN, Clermont-Ferrand (FR)

(72) Inventors: Victor MISHIN, Philadelphia, PA (US); Stephanie REED, Conshohocken, PA (US); Mohamed ELKHODIRY, Lansdale, PA (US); Jinal POTHUPITIYA, Warrington, PA (US); Manasi BAKER, Ambler, PA (US); Scott RADZINSKI, Gilbertsville, PA (US)

(21) Appl. No.: 18/784,056

(22) Filed: Jul. 25, 2024

Related U.S. Application Data
(60) Provisional application No. 63/515,399, filed on Jul. 25, 2023.
(60) Provisional application No. 63/515,402, filed on Jul. 25, 2023.
(60) Provisional application No. 63/515,403, filed on Jul. 25, 2023.

(51) Int. Cl. C08G 63/91; A61K 31/573; A61K 31/7076; A61K 47/34; C08G 18/42; C08G 18/73; C08G 18/77; C08G 63/16; C08G 63/78; C08J 3/24

(52) U.S. Cl. CPC C08G 63/916; A61K 31/573; A61K 31/7076; A61K 47/34; C08G 18/4238; C08G 18/73; C08G 18/771; C08G 63/16; C08G 63/78; C08J 3/24; C08G 2230/00; C08J 2367/02

(57) ABSTRACT

A composition includes a polyester urethane. The polyester urethane includes a crosslinker based on diisocyanate and an alternating copolymer resin of polyol and polyacid. The resin has a degree of branching, weight average molecular weight, polydispersity index, and viscosity prior to the crosslinking. The polyester urethane has a microstructure of hard segments and soft segments based on a selected combination of polyol, polyacid, degree of branching, the weight average molecular weight, the polydispersity index, the viscosity, the diisocyanate, the first stoichiometric ratio, and the second stoichiometric ratio. The microstructure provides a predetermined property to the composition selected from a degradation rate in an aqueous environment, release rate of a drug loaded in the polyester urethane in the aqueous environment, solubility of the drug, and combinations thereof.

### REFERENCE TO RELATED APPLICATIONS

This application claims priority to and the benefit of U.S. Provisional Application No. 63/515,399, U.S. Provisional Application No. 63/515,402, and U.S. Provisional Application No. 63/515,403, all filed Jul. 25, 2023, which are hereby incorporated by reference in their entirety.

### FIELD OF THE INVENTION

The present disclosure is generally directed to processes of forming urethane-containing polymers and compositions formed by such processes. More specifically, the present disclosure is directed to processes of forming polyester urethane (PEU) polymers with tunable degradation and controlled release rates and compositions formed by such processes.

### BACKGROUND OF THE INVENTION

The majority of biodegradable biomaterial polymers used for drug delivery are bulk eroders that exhibit a dose-dependent active pharmaceutical ingredient (API) release rate, where increasing the drug loading concentration increases the relative release rate. With such polymers, achieving a high drug loading that also provides sustained release for greater than three months is challenging, because the increased loading also generates a steeper concentration gradient between the polymer matrix and the surrounding environment. That, in turn, drives release to occur faster. Hence, for bulk eroders, such as poly(lactic-co-glycolic acid) (PLGA), polyglycolic acid (PGA), polylactic acid (PLA), or polycaprolactone (PCL), the release rate is often sufficiently low to achieve greater than three months of controlled release therapy when the loading is about 40 wt % or less, but loadings of about 50 wt % or greater often exhibit significantly faster release rates and thus often only provide at most one month of controlled release therapy.

This same limitation occurs with non-degradable polymers, such as poly(ethylene-co-vinyl acetate) (EVA), polyurethane (PU), and silicone, since bulk eroding and non-eroding drug delivery systems are both diffusion-driven. This is demonstrated, for example, in Barrett et al. (“Extended Duration MK-8591-Eluting Implant as a Candidate for HIV Treatment and Prevention”, Antimicrob. Agents Chemother., Vol. 62, Issue 10, 2018), where EVA, PCL, and PLA showed a steep increase in release rate as the drug loading increased from 40 wt %, to 50 wt %, to 60 wt %, and to 80 wt %. At 60 wt % loading, release from all three polymers was only two months in duration. At 80 wt % loading, the release duration dropped to one month in duration. Moreover, bulk eroding polymers often demonstrate dose dumping once a critical mass loss has been reached.

While release rate may be highly dependent on the solubility of the API, it would be highly advantageous to have a polymer carrier that is capable of delivering APIs across a solubility spectrum in a sustained manner, for at least three months and potentially many months longer. Highly soluble APIs pose a challenge to non-degradable and bulk eroding polymers, since Biopharmaceutical Classification System (BCS) class I (high solubility, high permeability) and class III (high solubility, poor permeability) APIs are likely to rapidly diffuse away from the polymer matrix, causing a large burst release and fast release rate. On the other hand, poorly soluble APIs also pose a challenge to non-degradable and bulk eroding polymers, since BCS class II (low solubility, high permeability) and BCS class IV (low solubility, low permeability) APIs have a difficult time diffusing away from the polymer matrix. Sufficient release rates typically cannot be achieved, especially within a reasonable timeframe after implantation. The majority of new drug entities developed by the pharmaceutical industry are BCS class II and IV, and so solubility and permeability concerns are becoming increasingly important to manage for effective controlled drug delivery. However, BCS class I and III APIs are still very much of interest for controlled release as well. Thus, having a polymeric delivery system that can deliver both highly-soluble and poorly-soluble APIs in the form of a matrix that is essentially agnostic to the API would be desirable. Further, having a polymeric delivery system that does not solely rely on diffusion, but instead releases the API through surface erosion, either in combination with diffusion or by surface erosion alone, is also highly desirable.

Poly(glycerol sebacate) urethane (PGSU) includes the building blocks glycerol, sebacic acid, and diisocyanate. Specifically, PGSU composed of glycerol, sebacic acid, and hexamethylene diisocyanate (HDI) has been crosslinked at a relatively higher crosslink density, such as 1:1.25 to 1:0.25 isocyanate-to-hydroxyl stoichiometric ratio. This higher crosslinking affords PGSU many useful properties, such as shelf stability at room temperature, low extractables and leachables, excellent biocompatibility, and mechanical robustness. This composition of PGSU is quite successful at achieving sustained release of many different small molecule drugs, or APIs. APIs with various solubilities, hydrophilicity/hydrophobicity, molecular weights, log(P) value, pKa, particle size and morphology, surface energy, and charge can be delivered from PGSU implants, which can provide a sustained release lasting for 3 months or longer, even as long as 24 months in some cases.

Conventional compositions of PGSU include a poly(glycerol sebacate) (PGS) soft segment crosslinked with HDI. PGSU degradation occurs after water interacts with ester bonds in the soft segment leading to bond breakage via hydrolysis, with the potential for water to also interact with and hydrolyze the urethane bonds in the hard segment, albeit on a much longer and slower time scale, and eventually the dissolution of polymer degradants. Given how much slower and less likely it is to hydrolyze urethane bonds compared to ester bonds, one way to formulate polyester urethanes for faster degradation is to have fewer urethane bonds. Some existing strategies employed to tune the degradation kinetics of these networks rely on changing the crosslink density, or isocyanate-to-polyol ratio. However, altering the crosslink density directly affects the polymer mesh size, drug release kinetics, shelf stability, durability, strength, extractables and leachables profile, biocompatibility, and surface versus bulk erosion properties. Another existing strategy includes varying the curing conditions and the length of a diacid copolymerized with erythritol (see, for example, Barrett et al., “Aliphatic polyester elastomers derived from erythritol and α,ω-diacids”, Polym. Chem., Vol. 1, pp. 296-302 (2010)).

Conventional PGSU compositions contain a soft segment with ester bonds that are separated by the eight-carbon methylene chain of the sebacic acid molecule. The breakage of one ester bond increases the likelihood of the neighboring ester bond breaking, because hydrolysis products, specifically acidic degradation products, can catalyze adjacent hydrolysis reactions. An equivalent network with shorter carbon chains between adjacent ester bonds, in other words a network with a higher ester density, or richer in catalytic products can increase the hydrolysis rate in the network. Hence, designing polymer networks with varying degrees of domains rich in polyacids creates a network with tunable hydrolysis rates.

Substituting sebacic acid with a shorter polycarboxylic acid to create new hyperbranched polyesters with the glycerol-polyacid repeating units but with different degradation profiles has been previously described (see, for example, Zhang et al., “Synthesis and characterization of glycerol-adipic acid hyperbranched polyesters”, Polymer, Vol. 55, pp. 5065-5072, 2014). Others have also synthesized copolymers combining sebacic acid and succinic acid to make a branched copolymer including both polyacids (see, for example, Godinho et al., “Synthesis of Prepolymers of Poly(glycerol-co-diacids) Based on Sebacic and Succinic Acid Mixtures”, ACS Omega, Vol. 18, pp. 16194-16205, 2023).

Conventional PGSU with approximately 1:1 to 1.1:1 isocyanate-to-hydroxyl stoichiometric ratio has a degradation time in the range of 12-36 months, depending on device geometry and anatomical position. Recent data on such PGSU, when tested across multiple in vitro and in vivo data sets, showed that it degrades in about two years as an unloaded construct and in about two to three years as a 40-60% loaded construct, depending on the geometry of the construct. For example, a 40%-loaded, 457-micron diameter microrods fully degraded in 20-24 months in vitro. In contrast, a 1-mm diameter rod at 50% loading with an extremely hydrophobic API, still held its shape after 2.6 years with twice-weekly media exchanges. Existing conventional compositions of PGSU cannot degrade in shorter timeframes without changing the crosslinking ratio and affecting the benefits listed above, even though various solvation, porogen, and dimension strategies have been tried. Therefore, there is a need for different ways of tuning polymer degradation rates to match different therapeutic applications without altering the crosslink density. Even a conventional PGS resin, without any crosslinking, does not readily degrade within a suitably fast enough timeframe for many applications, highlighting the need for an improved polyester resin composition beyond conventional PGS that can hydrolyze faster.

For controlled drug release applications, it is very desirable for drug release kinetics to synchronize with polymer degradation kinetics. This avoids the polymer lingering in situ after the drug payload has already been depleted. Therefore, it is desirable to have polymer compositions that can degrade at faster rates, to match various drug release rates. More specifically, it is desirable to achieve polymer degradation life spans as short as 1 month, 3 months, 6 months, 9 months, 12 months, and up through 18 months.

While reducing the crosslink density may be one approach to achieve faster degradation, simply reducing the crosslink density of PGSU by reducing the urethane-polyol ratio, or the isocyanate-to-hydroxyl stoichiometric ratio (NCO:OH mol:mol), however, does not lead to favorable performance outcomes. Reducing the isocyanate-to-hydroxyl stoichiometric ratio below 1:1.25, for example to 1:2.5, 1:3.5, and 1:4.5, results in a bulk degradation mechanism. Bulk degradation may be less favorable for sustained release compared to surface erosion, due to erratic, less linear, and less predictable polymer chain scission and drug release. Bulk degradation occurs when water infiltration out-paces the rate of polymer bond hydrolysis. Moreover, reducing the isocyanate-to-hydroxyl stoichiometric ratio below 1:1.25 results in very poor release kinetics. In addition, the degradation profile of these less-crosslinked PGSU formulations appear to be solubility limited, due to the low aqueous solubility of sebacic acid (0.25 mg/mL) and size limitation of PGS oligomers containing glycerol and sebacic acid (insoluble above 1000 Da), which leads to slower than expected mass loss even for lower crosslinking. This is a bigger issue for implants intended for anatomical positions with low fluid volumes and low fluid flow conditions, for example ocular or subcutaneous implants.

When a biodegradable polymer implant degrades, it breaks down into biodegradants of smaller molecules, oligomers, or fragments that separate from the bulk polymer, solubilize into surrounding fluid, and can be eliminated from the body. Increasing the biodegradant solubility can result in a more favorable biodegradable implantable polymer, because it allows for more control over the rate and mechanism of degradation. During hydrolysis, the movement of the fragments away from the bulk polymer relies on the aqueous solubility of the fragments. Even the movement of fragments within the bulk polymer is improved if the fragments are water-soluble, as this imparts mobility that allows the fragments to escape and eventually leave the bulk, which provides porosity to form and causes a more open mesh size and hydrolysis susceptibility of the remaining polymer bulk. If the fragments have low solubility in water, they cannot evacuate from the bulk, and future water infiltration and access can be blocked, causing the rate of degradation to be low. If the polymer includes acidic components, the degraded fragments containing acid functionality need to be water soluble in order to acid-catalyze further degradation. Moreover, if the fragments are not very soluble in the body's natural aqueous-based fluids, they will not easily come into contact with the enzymes or immune cells that can further break them down. Taken together, it is not enough to have cleavable sites within the polymer network, but rather the cleaved fragments need to evacuate, and fragment evacuation is improved if the fragments have aqueous solubility.

Biodegradable urethanes generally degrade via a three-step process that includes water diffusion into the polymer, hydrolysis and bond cleavage, and degradant dissolution. Conventional compositions and methods to create PGSU control the degradation kinetics primarily by changing the ratio of crosslinker to prepolymer to change the crosslinking density in the resulting PGSU. This conventional approach, although effective in creating a composition that loses mechanical strength faster, does not sufficiently hasten mass loss. Instead, this conventional approach reaches solubility limitations in the degradant dissolution step of polymer degradation. This limits the ability to tune polymer degradation especially in therapeutic in vivo applications, where the polymer is not suspended in an excess of fluid volume and flow as in common in vitro test conditions. Additionally, this negatively impacts drug release from API-loaded biodegradable urethanes, because the primary mechanism for release becomes diffusion instead of surface erosion when degradation kinetics are too slow.

Conventional compositions of PGSU break down to form biodegradants with low water solubility. Conventional PGSU formulations can often have a significant lag time between the loss of mechanical integrity and polymer dissolution, mass loss, and dimensional loss. This lag time can make the overall degradation process less controllable and the device lifetime and properties less predictable, especially depending on the fluid flow properties in the anatomical position of the polymer implant. Degradants with better water solubility can be more easily and quickly hydrolyzed, since water can access the degradants' molecular structure and hydrolytic cleavage sites with greater freedom when solubilized. Less soluble degradants may also require more breaks to reach small enough fragments that are water soluble. Slow biodegradation can be a disadvantage in applications where the material needs to degrade quickly, such as in a biomedical, pharmaceutical, veterinary, cell culture, diagnostic, bioprocessing, cosmetic, personal care, industrial, agricultural, or environmental applications. Less soluble degradants may accumulate in the environment or in a biological system, leading to potential toxicity, inflammation, or other harmful effects; may be more likely to cause inflammation or an immune response, which can limit the usefulness of such polymers in medical applications or other situations where biocompatibility is important; and may be more difficult to process and handle during manufacturing or disposal, which can increase costs, logistical challenges, and environmental impact. The generation of less soluble degradants may limit the potential uses of the biodegradable polymer, particularly in applications where solubility is important, such as drug delivery or other medical applications. It may also limit the use of the polymer to anatomical locations with high levels of fluids and fluid flow conditions, with high and low pH, with high enzyme concentration and activity, with an abundance of phagocytic cell types to help clear out the less soluble degradants, and with nearby lymphatic drainage.

Taken together, this indicates the need for more sophisticated approaches to polymer design, to achieve polyester urethanes that degrade more quickly but still sustain drug release.

### BRIEF DESCRIPTION OF THE INVENTION

In some embodiments, a composition includes a polyester urethane. The polyester urethane includes a crosslinker based on a diisocyanate and an alternating copolymer resin of at least one polyol monomer and at least one polyacid monomer. The crosslinker crosslinks the alternating copolymer resin. The alternating copolymer resin has a degree of branching, a weight average molecular weight, a polydispersity index, and a viscosity prior to the crosslinking. The polyester urethane has a first stoichiometric ratio of the at least one polyol to the at least one polyacid. The polyester urethane has a second stoichiometric ratio of isocyanate-to-hydroxyl. The polyester urethane has a degree of phase-separated microstructure between hard segments of the crosslinker and soft segments of the alternating copolymer resin. The crosslinking stabilizes the degree of phase-separated microstructure. The degree of phase-separated microstructure is based on a selected combination of the at least one polyol monomer, the at least one polyacid monomer, the degree of branching, the weight average molecular weight, the polydispersity index, the viscosity, the diisocyanate, the first stoichiometric ratio, and the second stoichiometric ratio. The degree of phase-separated microstructure provides a predetermined property to the composition selected from the group consisting of a degradation rate in an aqueous environment, a release rate of a drug loaded in the polyester urethane in the aqueous environment, a solubility of the drug, and combinations thereof.

In some embodiments, a process of forming a polyester urethane includes selecting at least one polyol monomer, at least one polyacid monomer, and a diisocyanate. The process also includes combining the at least one polyol monomer and an aqueous liquid in a vessel. The process further includes selecting a first stoichiometric ratio of the at least one polyol monomer to the at least one polyacid monomer and adding the at least one polyacid monomer to the vessel at the first stoichiometric ratio. The process further includes removing water from the vessel and producing an alternating copolymer resin of the at least one polyol monomer and the at least one polyacid monomer. The alternating copolymer resin has a degree of branching, a weight average molecular weight, a polydispersity index, and a viscosity. The process also includes selecting a second stoichiometric ratio of isocyanate-to-hydroxyl and homogenously combining a flowable blend including the alternating copolymer resin with the diisocyanate at the second stoichiometric ratio to form the polyester urethane. The at least one polyol monomer, the at least one polyacid monomer, the diisocyanate, the first stoichiometric ratio, the second stoichiometric ratio, the degree of branching, the weight average molecular weight, the polydispersity index, and the viscosity are selected to form a degree of phase-separated microstructure between hard segments of the crosslinker and soft segments of the alternating copolymer resin. The degree of phase-separated microstructure provides a predetermined property selected from the group consisting of a degradation rate in an aqueous environment, a release rate of a drug loaded in the polyester urethane in the aqueous environment, a solubility of the drug, and combinations thereof.

In some embodiments, an implantable product includes a drug and a polyester urethane. The polyester urethane includes a crosslinker based on a diisocyanate and an alternating copolymer resin of at least one polyol monomer and at least one polyacid monomer. The crosslinker crosslinks the alternating copolymer resin. The alternating copolymer resin has a degree of branching, a weight average molecular weight, a polydispersity index, and a viscosity prior to the crosslinking. The polyester urethane has a first stoichiometric ratio of the at least one polyol to the at least one polyacid. The polyester urethane has a second stoichiometric ratio of isocyanate-to-hydroxyl. The polyester urethane has a degree of phase-separated microstructure between hard segments of the crosslinker and soft segments of the alternating copolymer resin. The crosslinking stabilizes the degree of phase-separated microstructure. The degree of phase-separated microstructure is based on a selected combination of the at least one polyol monomer, the at least one polyacid monomer, the degree of branching, the weight average molecular weight, the polydispersity index, the viscosity, the diisocyanate, the first stoichiometric ratio, and the second stoichiometric ratio. The degree of phase-separated microstructure provides a predetermined property to the composition selected from the group consisting of a degradation rate in an aqueous environment, a release rate of the drug loaded in the polyester urethane in the aqueous environment, a solubility of the drug, and combinations thereof.

### BRIEF DESCRIPTION OF THE DRAWINGS

FIG. 1 shows a plot of conversion of glycerol, sebacic acid, and adipic acid during polycondensation to form poly(glycerol sebacate-co-adipate) at a monomer stoichiometric feed ratio of 1:0.5:0.5.

FIG. 2 shows global evolution of monomers, polymers, and microstructures during polycondensation to form poly(glycerol sebacate-co-adipate).

FIG. 3 shows a plot of the degree of branching of polyester resins versus the relative molar ratio of 1,2,3-triacylglyceride.

FIG. 4 shows a plot of the weight-average molecular weight of polyester resins versus the relative molar ratio of 1-acylglyceride.

FIG. 5 shows a plot of the weight-average molecular weight of polyester resins versus the relative molar ratio of 1,2,3-triacylglyceride.

FIG. 6 shows a radar plot of polyester urethanes having polyester components formed from polyesters composed of various molecular weights.

FIG. 7 shows a radar plot of PGSU formulations made using polyesters with either a 1:1 or a 1.1:1 polyol:polyacid molar ratio and either HDI or lysine diisocyanate ethyl ester (LDI).

FIG. 8 shows a radar plot of PGSU formulations made using polyesters with either a 1:1 or a 1.1:1 polyol:polyacid molar ratio and with HDI.

FIG. 9 shows a radar plot of PGSU formulations made using polyesters with either a 1:1 or a 1.1:1 polyol:polyacid molar ratio and with LDI.

FIG. 10 shows a radar plot of PGSU formulations made using polyesters with sebacic acid and either adipic acid or succinic acid and with HDI.

FIG. 11 shows a radar plot of PGSU formulations made using polyesters with either adipic acid or polyacid copolymers of adipic acid with sebacic acid at adipic acid:sebacic acid molar ratios of 50:50 or 25:75 and with HDI.

FIG. 12 shows a radar plot of PGSU formulations made using polyesters with either adipic acid or sebacic acid, either a 1:1 or 1.1:1 a polyol:polyacid molar ratio, and with HDI.

FIG. 13 shows degradation time at 70° C. versus molecular weight of HDI-crosslinked polyester urethanes.

FIG. 14 shows degradation time at 70° C. versus molecular weight of LDI-crosslinked polyester urethanes.

FIG. 15 shows degradation percentage after four weeks at 70° C. versus crosslink density for LDI-crosslinked polyester urethanes.

FIG. 16 shows degradation percentage after four weeks at 70° C. versus crosslink density for polyester urethanes doped with free acid.

FIG. 17 shows degradation percentage after four weeks at 70° C. versus crosslink density for HDI-crosslinked polyester urethanes.

FIG. 18 shows degradation percentage after four weeks at 70° C. versus crosslink density for polyester urethanes with a mixture of crosslinkers.

FIG. 19 shows sol content for certain polyester urethanes.

FIG. 20A shows the mass loss results for a selected group of polyester urethane implants.

FIG. 20B shows the diameter loss results for the polyester urethane implants of FIG. 20A.

FIG. 21 shows degradation profiles for polyester urethane implants.

FIG. 22 shows degradation profiles for additional polyester urethane implants.

FIG. 23 shows swell in tetrahydrofuran (THF) for selected polyester urethanes.

FIG. 24 shows swell in THF for additional polyester urethanes.

FIG. 25 shows swell in water for selected polyester urethanes.

FIG. 26 shows swell in water for additional selected polyester urethanes.

FIG. 27 shows water uptake for additional selected polyester urethanes.

FIG. 28 shows the crosslink densities of various polyester urethanes.

FIG. 29 shows the crosslink densities of a certain family of polyester urethanes.

FIG. 30A shows an SEM of a first polyester urethane implant prior to a three-month degradation study.

FIG. 30B shows an SEM of the polyester urethane implant of FIG. 30A after the three-month degradation study and drying by lyophilization.

FIG. 31A shows an SEM of a second polyester urethane implant prior to a three-month degradation study.

FIG. 31B shows an SEM of the polyester urethane implant of FIG. 31A after the three-month degradation study and drying by lyophilization.

FIG. 32 shows cumulative release profiles for a first set of polyester urethane microdevices loaded with 60 wt % dexamethasone.

FIG. 33 shows cumulative release profiles for a second set of polyester urethane microdevices loaded with 60 wt % dexamethasone.

FIG. 34 shows cumulative release profiles for a third set of polyester urethane microdevices loaded with 60 wt % dexamethasone.

### DETAILED DESCRIPTION OF THE INVENTION

Provided herein are polymer compositions including a polyester urethane with a microstructure of soft segments of generally amorphous polyester and hard segments of urethane crosslinking selected to provide a predetermined property to the polymer composition. Also disclosed herein are synthesis and formulation methods for changing the polyester urethane microstructure, separate from crosslink density, that can affect drug release and polymer degradation behavior. The microstructure of the soft segments and the hard segments is selected based on a recognition of their contribution, individually and collectively, on the resulting predetermined property of the resulting polyester urethane composition.

In exemplary embodiments, the crosslinking captures the microstructure of soft segments of polyester and hard segments of urethane crosslinking of the polyester urethane to provide a degradation and/or release character. In exemplary embodiments, the degradation and/or release character is different from what is achieved by a conventional PGSU.

In some embodiments, the crosslink density of a polyester urethane is tuned by changing the ratio of the soft segment polyol to the hard segment urethane, which consequently tunes the mechanical properties, release kinetics, and degradation kinetics.

In some embodiments, the polyester urethane includes a crosslinker based on a diisocyanate and an alternating copolymer resin of at least one polyol monomer and at least one polyacid monomer. The crosslinker crosslinks the alternating copolymer resin. The alternating copolymer resin has a degree of branching, a weight average molecular weight, a polydispersity index, and a viscosity prior to the crosslinking. The polyester urethane has a first stoichiometric ratio of the at least one polyol to the at least one polyacid. The polyester urethane has a second stoichiometric ratio of isocyanate-to-hydroxyl. The polyester urethane has a degree of phase-separated microstructure between hard segments of the crosslinker and soft segments of the alternating copolymer resin. The crosslinking stabilizes the degree of phase-separated microstructure. The degree of phase-separated microstructure is based on a selected combination of the at least one polyol monomer, the at least one polyacid monomer, the degree of branching, the weight average molecular weight, the polydispersity index, the viscosity, the diisocyanate, the first stoichiometric ratio, and the second stoichiometric ratio. The degree of phase-separated microstructure provides a predetermined property to the composition selected from the group consisting of a degradation rate in an aqueous environment, a release rate of a drug loaded in the polyester urethane in the aqueous environment, a Δ log(P) between the soft segments and the drug, a Δ log(P) between the soft segments and the hard segments, a Δ log(P) between the drug and the hard segments, a solubility of the drug, and combinations thereof.

Here, microstructure can be defined on at least two levels. One level is the microstructure of the polyester itself, which can be determined by ¹³C NMR, and which describes the branching, end groups, pendant functional groups, locations and frequency of esterification, proportions of these species, and overall architecture of the polyester. For example, when the polyol is a triol like glycerol and the polyacid is a diacid, various degrees of branching can occur, depending on which alcohol groups have formed an ester bond with a carboxylic acid, as shown schematically below for glycerol, where “P” represents a polymer chain. With two free alcohol groups, the glycerol is a 1-acylglyceride (1T), as shown by Formula (1), or a 2-acylglyceride (2T), as shown by Formula (2). With one free alcohol group, the glycerol is a 1,3-diacylglyceride (1,3L), as shown by Formula (3), or a 1,2-diacylglyceride (1,2L), as shown by Formula (4). With no free alcohol groups, the glycerol is a 1,2,3-triacylglyceride (1,2,3D), as shown by Formula (5).

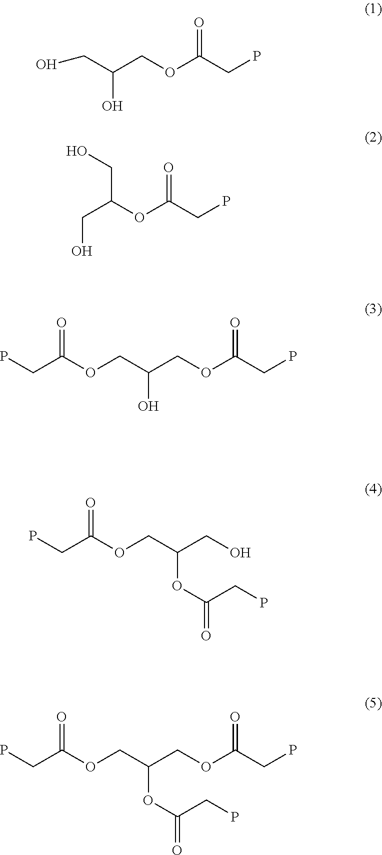

As used herein, the degree of branching is defined by Equation (1):

$$Degree of branching = 2 ( 1 , 2 , 3 D ) / ( 2 ( 1 , 2 , 3 D ) + 1 , 2 L + 1 , 3 L ) \qquad ( 1 )$$

In some embodiments, the degree of branching is selected to provide a predetermined property or set of properties to the polyester urethane. A decrease in branching in the polyester structure tends to increase the crystallinity of the matrix, which causes the melting temperature (Tm) to be higher. This is one of a number of features that may be tuned to provide a predetermined property or set of properties to the polyester urethane. Incorporation of short chain diacids increases the number of ester bonds to increase for a given weight average molecular weight (M_{w}), thus affecting the crystallinity and the hydrophilicity of the matrix. The shorter diacids also significantly increase the hydroxyl number, thus leading to inclusion of higher amounts of crosslinker and thereby increasing the crosslink density. Surprisingly, even though the crosslink density of matrices with short chain diacids were high, the degradation rates were significantly higher than with longer chain diacids.

A second level is how the polyester soft segment arranges relative to the urethane-crosslinked hard segment. The arrangement of this second polymer microstructure, specifically the degree of phase mixing, or segment mixing, between the hard segments and the soft segments of the polyurethane, has been shown to allow for increased hydrolytic degradation while sustaining or even improving release kinetics of hydrophobic APIs. The degree of phase mixing is dependent on the intermolecular interactions and thermodynamic stability of the soft and hard segments.

Arrangement of the soft segments and hard segments may include regions of only soft segments, regions of segment mixing, and/or regions of segment separation within polyurethane polymer structure. The hard domains, including the urethane crosslinking, can separate or mix with soft segments, including the polyesters, based on thermodynamic and chemical properties of the hard/soft segments. Agglomeration of distinct hard/soft domains, producing phase separation, can result in discrete drug release/degradation properties within the different segments. The overall microstructure and morphology of the polyester urethane can also depend on the interactions between the larger aggregated hard and soft segment domains. The presence of an API and the amount and properties of the API may also affect the arrangement of hard and soft segment domains.

The microstructural arrangement of hard and soft segments in a polyester urethane may be determined by one or more methods, which may include, but are not limited to, differential scanning calorimetry (DSC), thermal gravimetric analysis (TGA), Fourier transform infrared (FTIR) spectroscopy, X-ray diffraction (XRD), nanospectroscopy, or infrared nanospectroscopy, such as, for example, atomic force microscope infrared (AFM-IR) spectroscopy. Microstructure arrangement of drug particles within the polymer phase, such as, for example, drug particle dispersion and distribution, may be determined by one or more methods, which may include, but are not limited to, x-ray microscopy (XRM), such as, for example, micro-computed tomography (micro-CT) or nano-computed tomography (nano-CT), scanning electron microscopy (SEM), mosaic field of view (FOV) SEM, or energy dispersive x-ray analysis (EDX).

In some embodiments, a microstructure chemistry and arrangement is selected to obtain a desired drug release profile that is disconnected from the polymer erosion profile by way of diffusion-based release mechanisms.

In some embodiments, a microstructure chemistry and arrangement is selected to obtain a desired drug release profile that is disconnected from the polymer erosion profile by way of non-covalent interaction-based release mechanisms.

In some embodiments, a microstructure chemistry and arrangement is selected to obtain a desired drug release profile that is disconnected from the polymer erosion profile by way of drug particle dispersion and distribution.

In some embodiments, the microstructure of the polyester resin is selected based on the selection of a polyol, the selection of a polyacid, and the selection of a stoichiometric ratio of polyol-to-polyacid. In some embodiments, the polyester includes a chain extender. Apart from polyester resin and crosslinker chemistry imparting solubility to degradant fragments, chain extenders and other additives can be used to not only impart bonds that are prone to hydrolysis, but also chemical functionalities that promote interaction and greater solubility in water. Chain extenders and additives that are added to the polyester resin or during the formulation steps can contain hydrophilic functional groups that impart greater water solubility to the soft segment degradants upon hydrolysis. In some embodiments, the reaction conditions for forming and processing the polyester are also selected to achieve a predetermined microstructure of the polyester, such as, for example, a predetermined weight average molecular weight, number average molecular weight, polydispersity, degree of branching, glyceride species proportion and microstructure, acid species proportion and microstructure, acid value, hydroxyl value, free acid monomers, or free polyol monomers.

The ester bond spacing on the polyester resin remains fixedly unaffected by urethane crosslinking. When the polyol is glycerol (G), the ester bond spacing is a statistical average with spacing contributions from glycerol, where the two primary and single secondary hydroxyl groups can become ester crosslinks, and the polyacid, where the at least two carboxylic acids can become ester crosslinks. Sebacic acid (Seb) has a hydrocarbon (CH₂) length of eight, while adipic acid (Ad) has a hydrocarbon length of four, and succinic acid (Suc) has a hydrocarbon length of two. Copolymers of these different diacids have a combination of hydrocarbon lengths, which can be mole-averaged across the bulk material. For example, 50:50 mol % Ad:Seb has an average hydrocarbon length of six and 50:50 mol % Suc:Seb has an average hydrocarbon length of five. The ester bond spacing is influenced by how the glycerol and polyacid combine during synthesis, how the polymer chains build over time, and which hydroxyl sites on the glycerol participate in esterification. The propensity of glycerol's two primary hydroxyls versus its one secondary hydroxyl to participate in ester bond formation may differ during synthesis with different diacids. This leads to more or less branching, where a branch point is defined by a glycerol having each of its three hydroxyls forming an ester bond.

In some embodiments, a composition includes a polyester urethane that degrades in an aqueous environment into degradants that include degradants having a molecular weight greater than 1000 Da that are soluble in the aqueous environment. The polyester urethane itself is insoluble in the aqueous environment. In some embodiments, a polyester urethane includes a chemical composition and/or a chemical structure selected to form biodegradants having a higher solubility in an aqueous environment than conventional PGSU.

In some embodiments, the reference aqueous environment for determining the solubility of degradants of a polyester urethane or for comparing the solubility of degradants of two or more polyester urethanes is 0.1 M phosphate-buffered saline (PBS) at a pH of 7.4 and a temperature of 37° C. In other embodiments, the reference aqueous environment may be 0.1 M PBS at a pH of 7.4 and 70° C. In other embodiments, the reference aqueous environment may be 0.01 M PBS at a pH of 7.4 and 37° C., 0.9 wt % sodium chloride (saline) at 37° C., simulated gastric fluid (SGF) at a pH of 1.2, 1.8, or 5.5 and 37° C., or water at 37° C.

In exemplary embodiments, polyester urethane crosslinkers, additives, and/or polyester resins are selected that form polymer degradants with predetermined solubilities allowing for tunable degradation kinetics. The effects of these components on polymer degradant solubility independently and in combination with each other provides surprising and unexpected polymer characteristics. Creating more degradable polyurethanes has conventionally involved reducing the crosslinking density by reducing the isocyanate-to-hydroxyl stoichiometric ratio, which can lead to a reduced shelf-life, decreased mechanical integrity, worse sustained drug release, greater extractables and leachables, greater unreacted sol content, and/or more blooming of unreacted oligomers. Another conventional approach to forming faster-degrading biomaterials is to reduce the molecular weight, which can lead to decreased thermal stability, decreased mechanical integrity, worse sustained release, and/or more blooming of mobile oligomers. In some embodiments, a polyester urethane includes degradants having an increased solubility without a reduction in the crosslinking density and/or a reduction in the molecular weight.

In some embodiments, a polyester urethane polymer network with amphiphilic properties is created that contains both hydrophilic and hydrophobic components. These polymers can therefore have the benefits of a hydrophobic polymer, such as, for example, sustained drug release, surface erosion, and/or viscoelastic properties, while also overcoming solubility issues during degradation. The increased solubility seen in some larger molecular weight degradants, such as, for example, greater than 1000 Da, appears to be not simply the result of the hydrophilic components' solubility, but also the result of some small soluble amphiphilic fragments being able to solubilize larger fragments that are otherwise insoluble at such high molecular weights. In some embodiments, careful selection of the polymer network leads to the generation of soluble and solubilizable fragments during biodegradation, which can help to trigger solubilization of even larger sized molecules that independently would not be soluble. Without wishing to be bound by theory, this may be due to the entanglement and/or other non-covalent interactions of many smaller fragments that are more hydrophilic with larger chains, which may assist the solubilization of the larger molecules. In some embodiments, the more soluble polyester urethane networks described herein have a fragment solubility of up to 120 repeating units in length (see Example 21). This 30-fold increase cannot be solely attributed to simple component solubility or functional group addition, but also to the non-obvious interactions of the new small amphiphilic fragments with the larger insoluble fragments of the network. This effect is also a result of the polydispersity of polyester pre-polymer resins synthesized using a water-mediated process such as described in U.S. Pat. No. 9,359,472, which allows for more significant solubilization effects. Some resin formulations have a high proportion of lower molecular weight species, and even unreacted monomers, that participate in solubilization of the mix. In some embodiments, the resulting polymer network includes a hydrophobic soft-segment covalently linked to a hydrophilic hard segment (PGS-LDI), or vice versa in the case of incorporating shorter polyacids. In both cases, the degradants are amphiphilic molecules that can more readily dissolve in the neighboring aqueous environment at higher molecular weight/lower extent of degradation, resulting in more controlled polymer erosion and degradation.

In some embodiments, a polyester urethane bioresorbable elastomer with degradants having an enhanced solubility is formed with a polyester resin formed by a water-mediated polycondensation process having a polyol:polyacid molar ratio in the range of 0.9:1 to 1.4:1. The polyol and the polyacid may be added together at the same time, or in a step-wise, staged manner. Various reaction temperatures, times, and vacuum conditions may be used. Appropriate levels of free polyacid and free polyol monomers may include, but are not limited to, less than about 3 wt % each, alternatively less than about 2 wt % each, or any value, range, or sub-range therebetween. Appropriate acid values for the polyester resin may include, but are not limited to, less than about 75 mg KOH/g, alternatively less than about 60 mg KOH/g, alternatively less than about 50 mg KOH/g, alternatively more than about 35 mg KOH/g, or any value, range, or sub-range therebetween. Appropriate hydroxyl values for the polyester resin may include, but are not limited to, less than about 240 mg KOH/g, alternatively less than about 220 mg KOH/g, alternatively less than about 210 mg KOH/g, alternatively more than about 180 mg KOH/g, or any value, range, or sub-range therebetween.

For reference, copolymers of glycerol and sebacic acid only of four repeating units or less in length, corresponding to a molecular weight less than about 1000 Da, are generally soluble in an aqueous environment. Larger oligomers of glycerol-sebacic acid beyond a molecular weight of about 1000 Da are generally not water soluble. Degradants of conventional PGSU, which may include forms of their HDI crosslinker, beyond a molecular weight of about 1000 Da are also insoluble in an aqueous environment.

In some embodiments, a polyester urethane bioresorbable elastomer composition is formed with a predetermined average ester bond spacing. In some embodiments, a polyester urethane bioresorbable elastomer composition is formed with a predetermined hydrolysis rate selected while having an isocyanate-to-hydroxyl stoichiometric ratio that remains in the range of approximately 1:1 to 1.1:1. In other embodiments, the isocyanate-to-hydroxyl stoichiometric ratio is in the range of 1:5 to 1:0.25, alternatively 1:1.25 to 1:0.25, alternatively 1:1 to 1.1:1, or any value, range, or sub-range therebetween. In some embodiments, the composition includes building blocks or starting components that can result in a polymer network with a significantly higher hydrolysis rate than that of conventional PGSU. In some embodiments, combinations of building blocks change the hydrolysis rate in unexpected non-additive ways. In some embodiments, the resulting polyester urethane bioresorbable elastomer can hydrolyze down to small molecular weight biodegradant fragments in a time scale that is faster than conventional PGSU.

The prepolymers of polyester resin before crosslinking were evaluated by looking at an array of characteristics important for the downstream processing into pharmaceutical drug products and medical devices, including weight average molecular weight, number-average molecular weight, polydispersity, degree of branching, glyceride species proportion and microstructure, acid species proportion and microstructure, acid value, hydroxyl value, free polyacid monomers, free polyol monomers, residual moisture content, radius of gyration, hydrodynamic radius, glass transition temperature, melting temperature, crystallization temperature, viscosity, refractive index increment, miscibility with diisocyanates, miscibility with other prepolymers, and mixing with drug substances.

The polyol component has a significant impact on functional, mechanical, and degradation behaviors of crosslinked polyester urethanes. An appropriate polyol may include, but is not limited to, glycerol, ethylene diol, propylene diol, 1,4-butanediol, 1,5-pentanediol, hexanediol, heptanediol, octanediol, nonanediol, 1,10-decanediol, triethylene glycol, xylitol, poly(ethylene glycol), poly(ortho esters), or combinations thereof.

Specifically, use of a diol rather than a triol as the polyol may result in a more linear polyester resin due to reaction being limited to terminal hydroxyl groups. Alternatively, a polyol with additional functionality may be used to alter functional, mechanical, and degradation behaviors of crosslinked polyester urethanes. For example, changing the polyol component from glycerol to a polyol having greater than three hydroxyl groups, such as xylitol, which has five hydroxyl groups, may result in a more highly branched polyester resin structure.

The polyacid component of the polyester resin has a significant impact on functional, mechanical, and degradation behaviors of crosslinked polyester urethanes. More generally, changing from a linear polyacid to a polyacid with additional functionality can also change the behaviors of a crosslinked polyester urethane. An appropriate linear polyacid may include, but is not limited to, sebacic acid, suberic acid, adipic acid, succinic acid, itaconic acid, pimelic acid, or combinations thereof. Appropriate nonlinear polyacids may include, but are not limited to, citric acid. Other appropriate monomers may include, but are not limited to, acetic acid, glycolic acid, lactic acid, or combinations thereof.

In some embodiments, the polyacid is sebacic acid and a more hydrophobic network is formed, which may better retain a hydrophobic API and sustain its release. In other embodiments, the polyacid is less hydrophobic than sebacic acid and a less hydrophobic network is formed, providing for intermolecular interactions with a more hydrophilic API and sustaining its release. For example, when the drug is dexamethasone, the release rate is significantly different between when the polyol is sebacic acid and when the polyol is adipic acid. Thus, the composition of the polyester may be used to tune the release of different APIs with different degrees of hydrophilicity/hydrophobicity.

In some embodiments, the increased frequency of ester bonds, based on a shorter polyacid segment, allows faster degradation due to higher density of hydrolysis sites in a given volume. Appropriate shorter polyacids may include, but are not limited to, suberic acid, pimelic acid, adipic acid, glutaric acid, succinic acid, oxalic acid, itaconic acid, fumaric acid, maleic acid, or diglycolic acid. In some embodiments, the resulting polyester urethane network includes a soft segment with varying ester-to-ester lengths that controls the rate of hydrolysis and overall degradation without altering the ratio of soft to hard segments. In some embodiments, the increased solubility of byproduct degradants, based on a shorter polyacid segment, allows faster degradation due to degradants being easier solubilized in an aqueous environment and able to leave polymer construct network more easily. In some embodiments, the increased solubility of byproduct degradants, based on a particular isocyanate, allows faster degradation due to degradants being easier solubilized in an aqueous environment and able to leave polymer construct network more easily. These polymers may degrade in as quickly as three months or as slowly as three years. In all cases, the degradants are preferably small enough to be easily cleared by the body in vivo.

Depending on the polycondensation reaction conditions, different polyacids can incorporate at different rates, leading to polyester resins with different microstructures. Using shorter polyacids results in significantly faster reaction kinetics during polymer resin synthesis that are often harder to control using conventional poly-condensation reactions. This is an even more significant issue in reactions involving more than one polyacid in the case of copolymers. If the shorter polyacid reacts much faster with glycerol than the longer polyacid, the resulting copolymer has skewed monomer distribution and uncontrolled ratios. In other cases, the longer polyacid reacts more completely with glycerol than the shorter polyacid, perhaps due to chain flexibility. Shorter polyacids are also more water-soluble and hydrophilic, changing their reaction kinetics during poly-condensation, especially if using a water-mediated poly-condensation process. A water-mediated synthesis process similar to the process disclosed in U.S. Pat. No. 9,359,472 allowed sebacic acid and adipic acid to both incorporate well and at similar rates, while succinic acid incorporated less efficiently. This was a surprising result, given literature shows the opposite trend for non-water-mediated synthesis. These results were confirmed by ¹H-NMR and ¹³C-NMR analysis of microstructure, glyceride species distribution, residual monomer content, degree of polymerization, and degree of diacid incorporation (see, for example, Examples 3 and 4). Gel permeation chromatography (GPC) with a refractive index (RI) detector was used to further confirm residual monomer content, but gas chromatography-mass spectrometry (GC-MS) may be used as an alternative or additional technique for residual monomer quantification. The adipic-sebacic co-polymers likely have a different chain organization than succinic-sebacic co-polymers, given the difference in their rates of incorporation throughout polycondensation, and also given the difference in their residual monomers after polycondensation is complete.

How polyacid and polyol components react into the polyester network that builds during polycondensation likely has an appreciable impact on the resulting microstructure, free pendant functional groups, background functional groups, chain end functional groups, unreacted free monomers, and extractables and leachables. For example, a water-mediated synthesis of PGS with higher ratios of glycerol to sebacic acid lowers the number 1,2,3 triacyl glyceride units in the PGS polymer, thereby resulting in a less branched polymer.

In some embodiments, the flexibility of the soft segment polymer chains, based on a long polyacid component of the soft segment, improves sustained drug release. A long polyacid component here has a carbon chain length of eight or greater. For example, the use of sebacic acid with eight carbons and a linear structure improved dexamethasone sustained release over adipic acid or succinic acid with four and two carbons, respectively, also with linear structures. The long aliphatic chain backbone of sebacic acid allowed it more chain flexibility, compared to shorter polyacids like adipic acid and succinic acid. Sebacic acid may be able to coil or condense more freely due to its chain length, which can entrap drugs.

Various functional groups can be introduced in the polyester resin by way of the selected polyol or polyacid or an added chain extender that results in separation from the hard segment during crosslinking, creating distinct crystalline regions due to intermolecular interactions between the soft segment polymer chains. For example, a poly(glycerol sebacate) resin with an M_{w} of about 5,000 and a glycerol-to-sebacate molar ratio of about 1.1:1, crosslinked by HDI, and loaded with 60 wt % dexamethasone, shows significant separation between soft segments and hard segments. Alternatively, these interactions can be disrupted and weakened by introducing chemical moieties that preferentially interact with the hard segment, producing a phase-mixed microstructure with increased amorphous properties. Further, during the polyurethane formulation and crosslinking process, careful selection of crosslinker chemistry can produce segmented, or phase-separated, hydrophobic hard segment regions or alternatively can increase interactions, or phase mixing, with the soft segment.

In some embodiments, the frequency of free, unbound, pendant hydroxyl groups, based on a shorter polyacid segment in the polyester resin, allows more non-covalent interactions with drugs. In some embodiments, the frequency of ester bonds in the polymer backbone, based on a shorter polyacid segment, allows more non-covalent interactions with drugs. The length of the diisocyanate segment also plays a role in ester bond frequency. The diisocyanate may also contain free, unbound, pendant functional groups in its backbone.

In some embodiments, the frequency of amide or urethane bonds in the polymer, based on a shorter polyacid segment, allows more non-covalent interactions with drugs. The length of the diisocyanate segment also plays a role in amide or urethane bond frequency.

In some embodiments, the frequency of terminal end groups, based on lower molecular weight chains, allows more non-covalent interactions with drugs. The terminal end groups may be present in the polyester resin structure and carry through to still be present after urethane crosslinking. Alternatively, the terminal end groups may be revealed as the polyester urethane biodegrades. In some embodiments, the functionality of terminal end groups, whether hydroxyl or carboxylic acid, contributes to non-covalent interactions with drugs. The diamines generated from diisocyanate biodegradation also contribute as terminal end groups. In the case of LDI, biodegradation reveals a lysine amino acid containing an alpha-amino group, an alpha-carboxylic acid group, and a lysyl side chain. In the case of hexamethylene diisocyanate, biodegradation reveals hexamethylene diamine.

Beyond the frequency of these functional groups within the polymer network, the spatial arrangement is also important. The spatial arrangement is determined by polymer chain molecular weight and branching, first in the polyester resin architecture that results from polycondensation (OH groups reacting with COOH groups), and then in the network architecture that results from urethane crosslinking (OH groups reacting with NCO groups).

Another tunable parameter is how the hydroxyl groups of the polyol are reacted, either during polycondensation when they react with carboxylic acid groups to form ester bonds or during urethane crosslinking when they react with isocyanate groups to form urethane bonds. This highlights the importance of understanding and quantifying the hydroxyl value of the polyester resin, which indicates the free hydroxyl groups that are either pendant on the backbone or present on chain ends. Additionally, it is important to understand and quantify the available versus the reacted primary and secondary hydroxyls in the polyester resin. For example, a large number of secondary hydroxyls that remain unreacted during polycondensation (unconverted to ester crosslink by polyacid), are then available for urethane crosslinking (converted to urethane crosslinks by diisocyanate). A more linear polyester with low branching and correspondingly low 1,2,3-triglyceride content, such as less than 15 mol % 1,2,3-triglyceride, is a good example of this (see, for example, Examples 3 and 4). In this case, there were more unreacted secondary hydroxyls upon completion of polycondensation, and esterification more often occurred on the chain ends, extending the linear portion. This can give rise to a polymer network with ester linkages primarily along the backbone chain length and urethane linkages as branch points primarily orthogonal to the chain backbone. These architectural differences impact drug release, water interaction, and polymer degradants.

Generally, the polymer microstructure may be altered to enhance or reduce non-covalent interactions between the polymer backbone, polymer chain ends, and/or polymer pendant functional groups with drugs. Non-covalent interactions include electrostatic interactions, hydrophobic interactions, hydrogen bonding, pi-pi stacking, and dipole-dipole interactions. Hydrophobic interactions and hydrogen bonding are perceived as most relevant of these non-covalent behaviors for drug-loaded polyester urethanes.

For example, dexamethasone contains a fluorine atom, which is hydrophobic, polar, very electronegative, and electron dense. The electronegativity of fluorine is 3.98. Electronegativity describes the ability of an atom to attract and share electron pairs with another atom. The electronegativity of fluorine on dexamethasone likely causes it to interact with nearby polymer chain functional groups, such as the pendant hydroxyls, chain end hydroxyls, chain end carboxylic acids, backbone esters, backbone polyacid hydrocarbon chains, backbone diisocyanate hydrocarbon chains, backbone urethanes or amides, and biodegraded amines. Fluorinated compounds also have polar hydrophobicity. The polar hydrophobicity of fluorinated dexamethasone likely causes it to interact with nearby polymer chain functional groups, such as the pendant hydroxyls, chain end hydroxyls, chain end carboxylic acids, backbone esters, backbone polyacid hydrocarbons, backbone diisocyanate hydrocarbon chains, backbone urethanes or amides, and biodegraded amines. Dexamethasone likely participates in dipole interactions, hydrogen bonding, and hydrophobic interactions with the soft segments and/or hard segments. Furthermore, the steroid nucleus present in many corticosteroids, including, but not limited to dexamethasone, dexamethasone acetate, prednisolone, hydrocortisone, or betamethasone, has been shown to have affinity for acyl groups within a polymer backbone or pendant group. This affinity between acyl-based chemical structures within the polymer and the steroid nucleus of corticosteroids can be utilized to improve retention of such APIs within crosslinked polyester urethane polymer networks.

Other functional groups may be introduced as well, in the components incorporated during polycondensation reaction, in post-synthesis functionalization, in the components incorporated in the mixing step during urethane crosslinking, or in post-crosslinking functionalization, to achieve a desired interaction with a particular drug. Functional groups can be incorporated into the soft segment backbone, hard segment backbone, soft segment pendant groups, or soft segment chain ends. Appropriate components to include during polycondensation, to introduce new functionality, may include, but are not limited to, itaconic acid, compounds containing amines, methacrylates, or acrylates. An appropriate post-synthesis functionalization may include, but is not limited to, CLICK chemistry, acetylation of the hydroxyl groups, or tethering off polyesters from the hydroxyl pendants to make block copolymers using ring opening polymerization of cyclic lactones and thiolactones. Acetylation of the hydroxyl pendants may increase the lipophilicity or hydrophobicity of the soft segment allowing the reduction of the hydroxyl value giving more control over changing the crosslinking densities. Acetylated soft segments can be blended with non-acetylated soft segments followed by mixing with the crosslinking agent.

Appropriate additives to incorporate during the mixing step during urethane crosslinking may include, but are not limited to, PCL, PLGA, polyethylene glycol (PEG), compounds containing amines, maleimides, monofunctional isocyanates, or monofunctional isocyanates with other-ended functionality. Appropriate post-crosslinking functionalizations to introduce new functionality may include, but are not limited to, methacrylates, acrylates, cationic charges, anionic charges, PEG, primary, secondary, or tertiary amine groups, or alkyl chains. Post-crosslinking, bioconjugation chemistry schemes may be applied to any remaining free functional groups available in the polyester urethane, including, but not limited to, 1-ethyl-3-(3-dimethylaminopropyl)carbodiimide/N-hydroxysuccinimide (EDC/NHS), carbonyldiimidazole CDI, avidin-biotin, streptavidin-biotin, or CLICK chemistry.

In particular, monofunctional isocyanates are useful since their single-ended NCO contributes to the overall isocyanate-to-hydroxyl stoichiometric ratio, yet they do not form a double-sided bridge or crosslink in the network. Instead, they form a single-ended cantilever or dangling end where the other end is free to participate differently. This leads to an overall less crosslinked network, yet also the free end can contain a functionality designed to have a specific affinity for a particular drug. For example, a monofunctional isocyanate with an aliphatic chain or fatty acid tail may interact favorably and exhibit affinity with hydrophobic drugs like dexamethasone. Alternatively, a monofunctional isocyanate with a pendant unsaturated aromatic ring, or saturated six-carbon ring, or other bulky functional group may be used to disrupt packing or stacking or otherwise change the spatial arrangement of the polymer network. Alternatively, a monofunctional isocyanate with a hydrophilic pendant functional group may be used to improve water uptake or water swelling or affinity with hydrophilic drugs. In a similar sense, this can also be achieved with a diisocyanate species where one or more of the NCO functional groups are sterically hindered and do not participate as readily in forming crosslinks. An example of this is LDI, among others. The same number of moles of LDI as HDI were incorporated in formulations for crosslinking, yet LDI-crosslinked networks resulted in lower crosslink density than HDI-crosslinked networks. The reduced crosslinking of LDI may be a result of the difference in the reactivities of the two isocyanate groups in LDI. The 2-isocyanate group of ethyl (2S)-2,6-diisocyanatohexanoate is less reactive due to steric effects. This may lead to a portion of LDI that is only monofunctionally tethered into the network and not forming bifunctional crosslinks, due to steric hindrance on LDI's 2-isocyanate group. The unreacted isocyanate groups are most likely to convert to amine groups, that may also enhance the ability of LDI-containing matrices to hydrogen bond with the API. This may affect the rate of release of API.

Appropriate mono-functional isocyanates may include, but are not limited to, octyl isocyanate, butyl isocyanate, tolyl isocyanate, toluenesulfonyl isocyanate, chlorosulfonyl isocyanate, fluorophenyl isocyanate, 4-chloro-3-(trifluoromethyl)phenyl isocyanate, or (tert-butyl)phenyl isocyanate. Blends of mono-functional and di-functional isocyanates were tested, combining HDI with octyl isocyanate (OI) at various ratios of approximately 0.55:0, 0.4:0.3, 0.27:0.6, and 0.13:0.9 vol:vol HDI:OI (100:0, 75:25, 50:50, 25:75 mol:mol HDI:OI). Solid, clear, bubble-free PGSU elastomers were formed at all ratios, although the elastomers were increasingly softer with increasing OI and decreasing HDI. The crosslink density (mol/L) of these PGSU elastomers correspondingly decreased more than 10-fold, going from 100:0 to 25:75 mol:mol HDI:OI. This approach of combining mono- and poly-functional isocyanates can be a suitable way to achieve mechanically softer and faster degrading polyester urethane formulations.

Changing the stoichiometric ratio of the polyol to polyacid in the polyester resin, such as, for example, from 1:1 to 1.1:1 mol:mol, produces more hydrophilic chain terminations, increasing intermolecular interactions between the soft segment polymer chains and affecting functional behavior in terms of both degradation of the polymer and sustained release of loaded APIs. Release of APIs may be sustained by non-covalent interactions between the hydroxyl of the polyols and API. Non-covalent interactions may include, but are not limited to, hydrogen bonding, ionic interactions, hydrophobic interactions, or combinations thereof. The particle size of the API may also affect polymer degradation and API release rates. Furthermore, this 1.1:1 stoichiometric ratio may be used to tune the molecular weight of the polymer and may favor the synthesis of a more linear polyester resin due to primary terminal hydroxyl groups being more available and thermodynamically favored in the case of a triol such as glycerol. An excess of glycerol results in more primary hydroxyls reacting with the polyacid, and it is statistically less probable for secondary hydroxyls to react with the polyacid, rendering greater polyester linearity and more free secondary hydroxyls as pendant groups and/or urethane branch points.

A number of soft segment polyester resins were synthesized. Although 1:1 and 1.1:1 polyol:polyacid stoichiometric ratios were used, 0.9:1 polyol:polyacid stoichiometric ratios are also possible. In some embodiments, the stoichiometric ratio of the polyol to polyacid is changed, such as, for example, from 1:1 to 0.9:1 mol:mol, to produce a more polyacid dominant network. Furthermore, this stoichiometric ratio may favor synthesis of a polyester resin with more branching due to an excess of polyacid being able to target all the available hydroxyl groups on a triol such as glycerol.

In some embodiments, a catalyst, such as, for example, certain lipase enzymes, is included during polymerization that produces a very linear polyester resin.

In some embodiments, a chain extender is included in the polyester to increase the distance between the soft segments and thus increase the mesh size of the crosslinked polymer. This may increase or decrease microphase mixing between the hard and soft segments due to the thermodynamic incompatibility between segments and greater interphase interaction within the hard and soft segments. In some embodiments, chain extenders are incorporated into the hard segments by first reacting with and extending the diisocyanate. This ensures that the distance between two polyester resin chains is maintained constant while increasing the mesh size. Alternatively, the chain extender may be added to the side of PGS with the catalyst and not the side with isocyanate. This prevents the cross-reaction between chain extender and isocyanate.

An appropriate chain extender may include, but is not limited to, 1,4-butanediol, 1,5-pentanediol, 1,6-hexanediol, isopentyldiol, 2-methyl-1,3-propanediol, bis(2-hydroxyethyl) terephthalate, ethylene glycol, diethylene glycol, propylene glycol, dipropylene glycol, ethylene diamine, 2-hydroxyethyl 2-hydroxypropanoate, 2,2-bis(hydroxymethyl) propionic acid (DMPA), bis(2-hydroxyethyl) terephthalate (BET), dithiothreitol (DTT), poly(ortho esters), or a combination thereof. In some embodiments, diol-based or polyol-based components of various chain lengths and architectures can be incorporated into the soft segments during the polycondensation reaction with polyol and polyacid, alternatively upstream of polycondensation by first reacting with and extending the polyol, or alternatively upstream of polycondensation by selectively and partially reacting with the polyacid. An appropriate amount of chain extenders in the formulation may include, but is not limited to, up to 10 wt %, alternatively up to 7 wt %, alternatively up to 5 wt %, alternatively up to 2 wt %, or any value, range, or sub-range therebetween, of the total components of the formulation including the polyester resin, isocyanate, and any drug.

In some embodiments, a water-mediated polycondensation process similar to the process disclosed in U.S. Pat. No. 9,359,472 is used. In some embodiments, polyol and polyacid are added together at the same time. In other embodiments, polyol and polyacid are added in a step-wise, staged manner. Additionally, the reaction could be actively monitored for concentrations of various additives, such as polyols and polyacids, to determine the optimal timing for staged addition of reagents. A feedback loop can be implemented to have tighter control over stoichiometric ratios of polycondensation reaction additives. Various reaction temperatures, times, and vacuum conditions may be used to obtain predetermined results.

In some embodiments, the polycondensation reaction conditions are selected such that the polyester resin has a low M_{w}. An appropriate low M_{w} for the soft segment may include, but is not limited to, below 9000 Da, below 8000 Da, alternatively below 6000 Da, alternatively below 5000 Da, alternatively below 4000 Da, alternatively below 3000 Da, or any value, range, or sub-range therebetween. The corresponding number-average molecular weight (Mₙ) may be below 4000 Da, alternatively below 3000 Da, alternatively below 2000 Da, or any value, range, or sub-range therebetween.

In some embodiments, the polycondensation components and reaction conditions are selected such that the soft segment has low branching. As used herein, low branching refers to having less than 15 mol % of 1,2,3-triacylglyceride species as determined by ¹³C-NMR. In some embodiments, the soft segment has less than 14 mol % of 1,2,3-triacylglyceride species, alternatively less than 13 mol %, alternatively less than 12 mol %, or any value, range, or sub-range therebetween. As used herein, high branching refers to having greater than 15 mol % of 1,2,3-triacylglyceride species as determined by ¹³C-NMR.

In some embodiments, the polyester resin is synthesized by adding portions of the monomer components stepwise or dropwise or a combination thereof to tune the branching of the polymer chain. Adding the polyol in small portions or dropwise to the polyacid can lead to a more hyperbranched polyester resin chain, whereas adding small portions of the polyacid component to the polyol may lead to less branching in the polyester resin. A stepwise strategy, whereby the polyester resin is synthesized by first adding an excess of polyacid to the polyol followed by repeated alternating steps of adding a stoichiometric equivalent or excess of polyol and adding an excess of polyacid, may be used to create hyperbranched polymers.

In some embodiments, the polyester resin is dialyzed to remove residual free polyacid and residual free polyol prior to crosslinking. In some embodiments, dialysis is performed with 2000 Da cutoff membranes and methanol as the solvent. In some embodiments, dialysis is performed with 2000 Da cutoff membranes and isopropyl alcohol (IPA) as the solvent. In some embodiments, too much residual free polyacid leads to bubbles in the formulation, due to side reactions of isocyanate with acid.

In some embodiments, the polyester resin is dried to remove residual water content prior to crosslinking. In some embodiments, the drying occurs in a vacuum oven at about 30° C. to about 60° C. and about 10 torr for about 24 hours. In some embodiments, too much residual water content leads to bubbles in the formulation, due to side reactions of isocyanate with water. An appropriate level of residual water content may include, but is not limited to, less than about 0.1 wt %, alternatively less than about 0.05 wt %, or any value, range, or sub-range therebetween.

In some embodiments, the polyester resin is crosslinked with an isocyanate, at an isocyanate-to-hydroxyl stoichiometric crosslinking ratio in the range of about 1:1 to 1.1:1. In some embodiments, the polyester resin is melted at about 70° C. to ensure efficient catalyst incorporation and degassing despite the higher viscosities of some of the polyester resins. In some embodiments, the polyester resin is cooled to below 35° C. before adding and mixing in the crosslinker to reduce the likelihood of diisocyanate-free acid side reactions. In some embodiments, the speed mixer is also chilled prior to this step. In some embodiments, crosslinking is allowed to proceed for 24 hours before any further sample manipulation. In some embodiments, crosslinking is accelerated using a temperature increase and/or a catalyst to reduce the cure time prior to sample manipulation.

In some embodiments, the copolymer synthesis process to generate block, alternate, or random copolymers is varied to provide the effect of these microstructures on mechanical properties and release kinetics. In some embodiments, the varying affects both diffusion of water molecules into the polymer structure and interactions of chains with entrapped API molecules.

In some embodiments, the microstructure of the soft and hard segments of the polyester urethane is selected based on the selection of the polyester resin, the selection of the diisocyanate crosslinker, and the selection of a stoichiometric ratio of isocyanate-to-hydroxyl. In some embodiments, the diisocyanate includes a chain extender. In some embodiments, a free acid is added prior to crosslinking. In some embodiments, the reaction conditions for crosslinking are also selected to achieve a predetermined microstructure of the soft and hard segments.

In terms of mechanical properties, it was observed that the stiffness of the polyester urethanes correlated to the viscosity of the polyester resin, the degree of crosslinking, and the microphase behavior of the resulting polyester urethane. Incorporation of shorter chain polyacids, such as succinic and adipic acids, produced polyester resins with increasing viscosity as polyacid length decreased, for a given molecular weight. For example, an 8-kD poly(glycerol adipate) had a viscosity of 4.53 Pa·s, while an 8-kD poly(glycerol 50% sebacate-co-50% adipate) had a lower viscosity of 1.85 Pa·s. The longer chain length of sebacic acid imparts flexibility in the polymer backbone, and so shorter polyacids produce polymer chains with less mobility, less flexibility, and consequently higher viscosity. The increased viscosities may also be attributed to an increase in crosslinking via ester bonds between the polyester resin chains as the frequency of ester bonds increases with decreasing polyacid length. Furthermore, adipic acid-containing and succinic acid-containing polyurethane polymers were substantially stiffer once crosslinked into urethanes, with the same trend of increasing viscosity as the carbon chain length of the polyacid decreased. The longer chain length of sebacic acid imparts flexibility in the polymer backbone that carries through into the crosslinked urethane, and so shorter polyacids yield crosslinked urethanes with less flexibility and consequently increased stiffness. The increase in stiffness of the polyurethane material may also be attributed to increased intermolecular interactions and frequency of crosslinks between the polyacid groups within the soft segments. Various copolymer combinations of polyacids, such as, for example, succinic, adipic, and/or sebacic acid, such as, for example, 5-95 mol %, 10-90 mol %, alternatively 20-80 mol %, alternatively 25-75 mol %, alternatively 50-50 mol %, alternatively 75-25 mol %, alternatively 80-20 mol %, alternatively 90-10 mol %, alternatively 95-5 mol %, or any value, range, or sub-range therebetween, polyacid added to polyester resin synthesis by mol % may be used to tune the viscosity of the synthesized polyester resin based on the degrees of ester group crosslinking densities and polyacid chain length flexibility. In some embodiments, a polyester urethane includes a polyester of a copolymer of glycerol and a polyacid of adipic acid and sebacic acid at a molar ratio in the range of 5:95 to 95:5. In some embodiments, a polyester urethane includes a polyester of a copolymer of glycerol and a polyacid of succinic acid and sebacic acid at a molar ratio in the range of 5:95 to 80:20.

An appropriate polyisocyanate may include, but is not limited to, HDI (log(P) of 0.89, where log(P) is the log of the partition coefficient of HDI between octanol and water at infinite dilution), LDI (log(P) of 0.76), methylene diphenyl diisocyanate (MDI), toluene diisocyanate (TDI), isophorone diisocyanate (IPDI), tetramethylxylene diisocyanate (TMXDI), dodecamethylene diisocyanate (1,12-diisocyanatododecane or 12DI, theoretical log(P) of 6.4 and experimental log(P) of 3.559), lysine triisocyanate, (4-isocyanato-phenoxy)-acetic acid 2-[2-(4-isocyanato-phenoxy)-hexanoyloxy]-ethyl ester (log(P) of 1.28), 6-(4-isocyanato-phenoxy)-hexanoic acid 2-[6-(4-isocyanato-phenoxy)-hexanoyloxy]-ethyl ester (log(P) of 2.77), poly(propylene glycol) tolylene 2,4-diisocyanate terminated, benzoic acid 4-isocyanato-ethylene glycol bis lactic acid ester (log(P) of 1.64), poly(hexamethylene diisocyanate), 4,4′-methylenebis(cyclohexyl isocyanate) log(P) (H12MDI, of 5.48), 1,3-bis(isocyanatemethyl)cyclohexane (BIMC, log(P) of 3.92) aliphatic diisocyanates, aromatic diisocyanates, aliphatic-aromatic combination diisocyanates, blocked diisocyanates, polyisocyanates with hydrolyzable groups within the backbone such as acyl chloride, anhydride, or ester, or combinations thereof.

Beyond merely crosslinking the polyester resin, the selection of a polyisocyanate can affect a number of properties of the resulting polyester urethane. For example, the solubility of the degradants may be increased by changing from HDI to a different diisocyanate crosslinker. In some embodiments, the crosslinker assists degradation, regardless of the molecular weight or polydispersity of the polyester resin, reducing or eliminating the need for molecular weight limitations or compromises. For example, the resulting elastomer, when crosslinked with LDI, was found to degrade three times faster than conventional PGSU crosslinked with HDI. In addition, LDI-crosslinked elastomers degraded and fully solubilized while retaining their original shape, whereas conventional PGSU constructs, crosslinked with HDI, lost all mechanical properties and their original shape before fully degrading. In some embodiments, LDI-crosslinked polymers exhibit a lower crosslink density than HDI-crosslinked polymers when mixed at the same isocyanate-to-hydroxyl stoichiometric ratio. The LDI-crosslinked elastomers also generated degradants that were completely water soluble at much higher molecular weights than HDI-crosslinked elastomers, leading to faster degradation than HDI in all tested cases. The impact of LDI-crosslinking on drug release, however, did not follow a universal trend as discussed in further detail below.

For the final crosslinked polyurethane, the degree of microphase separation, reflected by the difference in the log of the partition coefficient between octanol and water (log(P)) of the crosslinker and the polyester resin (Δ log(P)), as well as the crystallinity of the hard and soft segments, resulted in polyurethanes with a range of viscoelastic behaviors. Increasing the degree of microphase separation imparts increased stiffness and strength. Mechanical stiffness is a good illustration of the effect of the differences in polyol microstructure even with equivalent urethane crosslinking based on the same isocyanate-to-hydroxyl stoichiometric ratio. This illustrates the effect of stabilizing the polyol microstructure using urethane crosslinking and preserving its physical effects. Crosslinker-mediated effects on solubility were seen when using a crosslinker that has a log of partition coefficient between octanol and water (log(P)) that is between 0 and 0.8. LDI has a log(P) of 0.76. More hydrophobic crosslinkers, including HDI, with a log(P) value between 0.8 and 6.5 were also tested. In one example, PGS-HDI took 15 weeks (about 105 days) to fully solubilize in aqueous media in an accelerated 70° C. study, and the resulting degradants had an overall Mw of 545 Da and an overall Mn of 387 Da. In contrast, PGS-LDI, made with the same polyester resin, took only 35 days to fully solubilize in aqueous media under the same conditions, and resulting degradants were much larger with an overall Mw of 13,004 Da and an overall Mn of 1,455 Da. This suggest that the PGS-HDI needed to experience many more hydrolytic chain scission events before the polymer network could eventually fully solubilize, whereas PGS-LDI could fully solubilize after far fewer hydrolytic events. LDI reduces the need to hydrolyze polymer bonds quickly and/or with high frequency, in order to degrade the polymer network. LDI helps larger polymer chains go into aqueous solution and helps to reach full solubilization. In another example, PGS crosslinked with 12DI demonstrated even slower degradation than conventional PGSU crosslinked with HDI.

Examples that reflect this behavior include the use of polyester resins with more hydrophilic polyacids and lower M_{w}, combined with a more hydrophilic crosslinker with a lower log(P) value, resulting in better phase mixing and significantly softer and more elastic polyurethanes. When the crosslinker was switched to more hydrophobic HDI with a higher log(P) value, the lack of pendant groups on the crosslinker, as well as the increase in log(P), resulted in a material with more microphase separation and crystallinity within the hard segment. When a more hydrophobic polyester resin, such as a high M_{w} PGS, was combined with LDI, the pendant lysine group on the crosslinker disrupted the packing of the hard segment, despite the increase in Δ log(P). This is a non-obvious phenomenon that may be used to control the degree of microphase separation to improve sustained drug release, while preserving desired mechanical properties. In another example, combining the same high M_{w} PGS with a larger, more hydrophobic, linear crosslinker, such as 12DI, resulted in a significantly stiffer and more crystalline polyurethane material, which also degraded more slowly than the same high M_{w} PGS crosslinked with HDI. An important observation here was the effect of functional groups and polarity of the hard segment on microphase separation. The use of isocyanate-based crosslinkers with low polarity and linear structures (HDI and 12DI) decreases the intermolecular interactions between the hard and soft segment, producing crystalline polyurethane materials with high degrees of microphase separation. On the other hand, isocyanate-based crosslinkers, such as LDI, with functionalities that result in hydrogen bonding and other interactions between the hard and soft segments decrease microphase separation. The resultant decrease in stiffness is exemplified by formulations of various PG:Seb-based and PG:Ad-based polyester resins combined with LDI. Once again, despite the decrease in stiffness of a formulation of PGS resin with LDI, the sustained release kinetics were improved, indicating a non-obvious interaction that can be attributed to a combination of ester crosslinks within the polyester resin, the crosslink density of the polyurethane, and the microphase interactions between the hard segments of urethane and soft segments of polyester.

Surprisingly, using different diisocyanates at the same molar ratio resulted in different crosslink densities, as determined by solvent swell test. Upon combination of the polyester resins with isocyanate-based crosslinkers, it was observed, also surprisingly, that the stiffness of the cured polyurethane material did not always correlate with the crosslink density as determined by solvent swell test. Both stiffness and crosslink density by solvent swelling are bulk physical measurements of the polymer mesh microstructure. The stoichiometric ratio of isocyanate-to-hydroxyl, on the other hand, is a description of the components added to the formulation but not necessarily a description of how the resulting polyester urethane is physically or chemically arranged or structured. All formulations were crosslinked with an isocyanate-to-hydroxyl stoichiometric ratio in the range of 1.1:1 to 1:1. Yet, a wide variety of crosslink densities were achieved. Also, a wide variety of mechanical properties were achieved. For a slower reacting crosslinker than HDI, such as LDI, a lower crosslink density (1.058 mol/L for LDI compared to 1.8-2.2 mol/L for HDI) resulted in softer, more elastic materials. The slower reactivity of LDI may be a result of steric hinderance due to the presence of the lysine group. When a larger, linear diisocyanate crosslinker (12DI) was used, the resultant material was glassier and stiffer despite reporting a lower crosslink density by swell test (1.252 mol/L). This was a non-obvious feature, which indicated the mechanical properties of the crosslinked polyurethane are not solely dependent on crosslink density. Another non-obvious feature was that the crosslink density was not necessarily dependent on the isocyanate-to-hydroxyl stoichiometric ratio.

Although the library of polymers outlined here can have a range of crosslink densities based on the isocyanate-to-hydroxyl stoichiometric ratio, it also provides an opportunity to tune polymer degradation without changing the ratio of crosslinker to hydroxyl groups. In such cases, this approach focused on increasing network hydrolysis by changing polyacid make up and produced constructs with more degradable timeframes without negatively affecting mechanical integrity, extractable and leachable levels, shelf life, crosslink density, or polymer mesh size. In some embodiments, the resulting crosslink density of the polymer network, based on the physical mesh size of the polymer, varies among the various soft segment and hard segment formulation combinations, despite a consistent crosslinking ratio of approximately 1:1 to 1.1:1 isocyanate-to-hydroxyl stoichiometric ratio being maintained during formulation mixing. This highlights that even when trying to control for equivalent molar proportions of isocyanate (NCO) and hydroxyl (OH) ingredients into the mix, the resulting urethane crosslinked microstructure is nuanced and depends on many factors. These factors may include, but are not limited to, properties of the component, such as hydrophobicity/hydrophilicity, polarity, molecular weight, branching, packing, radius of gyration, or spatial accessibility, properties of the formulation, such as free monomers or residual water content, and properties of the crosslinking reaction, such as efficiency of urethane reaction or rate of urethane reaction.

In some embodiments, the polyester resin and/or the diisocyanate is combined with a solvent prior to combining the polyester resin and the diisocyanate. An appropriate organic solvent may include, but is not limited to, acetone, propyl acetate, tetrahydrofuran (THF), ethyl acetate, butyl acetate, dichloromethane (DCM), dimethyl formamide (DMF), dimethylsulfoxide (DMSO), methanol, ethanol, IPA, or a combination thereof. In some embodiments, the solvent is a mixture of acetone and propyl acetate at 50:50, or 10:90, or 25:75, or 75:25, or 90:10 v:v %, or a sub-range therebetween. An appropriate amount of solids, when the solvent is added to the soft segment polymer, may include, but is not limited to, 10 to 90 wt % solids, alternatively 40 to 60 wt % solids, alternatively 40 to 80 wt % solids, or any value, range, or sub-range therebetween, with the remaining amount solvent. In other embodiments, the polyester resin and the diisocyanate are combined in the absence of a solvent, and the polyester urethane formulation includes a polyester resin, a crosslinker, optional additives such as chain extenders or free acids, and optional APIs, where the soft segment, and the entire formulation, is free of solvent.

In some embodiments, a free acid additive is included prior to crosslinking. In some embodiments, the free acid increases the hydrolysis rate of the polyester urethane through acid-catalyzed hydrolysis. Appropriate free acids may include, but are not limited to, tartaric acid, acetic acid, citric acid, glycolic acid, or lactic acid. In some embodiments, a solid free acid is mixed into the polyester resin immediately after melting to ensure a more homogenous distribution. In some embodiments, the free acid additive is maintained in the solid state as a dispersion to avoid cross-reaction and ensure the free acid can later solubilize and catalyze.

Instead of including shorter polyacids in the polyol synthesis, free acids that were short in length were added to the formulation during the urethane crosslinking step and remained in the network as catalysts to the hydrolysis process to accelerate degradation. These free acids could be monoacids, diacids, or polyacids. In particular, DL-tartaric acid had unique effects on the degradation kinetics of PGSU compared to acetic acid, citric acid, and lactic acid. Free acid could be added from 0.1 wt % up to 1 wt % without negative consequences to the properties of the formulation, such as, for example, side reactions causing carbon dioxide bubbles, and tartaric acid specifically could be added up to 5 wt % and still produce good polyester urethane properties. Adding as little as 0.1 wt % of tartaric acid to the formulation increased mass loss of PGSU from 24% to 55% after 4 weeks in aqueous media under accelerated (70° C.) conditions. In addition, free tartaric acid, unlike other free polyacids, was incorporated in the crosslinked polymer network at up to 5 wt % without any noticeable gas bubbles produced from acid-diisocyanate side reactions that were observed with the other acids even as low as at 1 wt %. This may be because tartaric acid was incorporated as a solid, as opposed to a liquid, during mixing into the formulation. It has been shown that additives or drugs, when mixed into a formulation as solid insoluble particles, do not easily cross-react with isocyanate during urethane crosslinking. In contrast, acid additives that were added to PGSU in liquid form may have cross-reacted with isocyanate as a side reaction, leading to the formation of carbon dioxide, which can present as bubbles.

In some examples, HDI-crosslinked networks degraded faster when tartaric acid was incorporated into the formulation as a free acid, due to acid-catalyzed hydrolysis, but surprisingly LDI-crosslinked networks did not degrade faster when tartaric acid was present. This may indicate a specific and selective relationship between HDI and tartaric acid that works well for acid-catalyzing hydrolysis.

In some embodiments, hard segments offer mechanical strength while soft segments contribute to flexibility of the polyester urethane material. The approaches described herein can be adapted to increase the degree of microphase mixing by increasing intermolecular interactions between hard and soft segments, resulting in a less crystalline, softer, more flexible material applicable for soft, biodegradable polymeric coatings. In other embodiments, microphase separation can, in contrast, be increased, leading to distinct regions of crystallinity between hard and soft segments, increasing the mechanical strength of material for applications that require support of prolonged, cyclical use.

In some embodiments, the polyester urethane is loaded with a drug or API for controlled release of the drug or API, with the drug or API being added either during polyester resin formation or during the crosslinking with diisocyanate. Appropriate APIs may include, but are not limited to, dexamethasone, triamcinolone acetonide, fluocinolone acetonide, bimatoprost, risperidone, naproxen, ibuprofen, nepafenac, amfenac, pyrantel pamoate, trenbolone acetate, or a prodrug thereof, non-steroidal anti-inflammatory drugs (NSAIDs), such as, for example, naproxen, ibuprofen, amfenac, or a prodrug thereof, steroids, hormones, prostamides, antibiotics, anti-oxidants, anti-viral drugs, anti-retroviral drugs, anti-psychotic drugs, and other drug classes. Appropriate APIs may include small molecules, peptides, proteins, antibodies, oligonucleotides, ribonucleic acid (RNA), deoxyribonucleic acid (DNA), cytokines, and growth factors.

In some embodiments, a spatial microstructural arrangement creates a delayed-release mechanism through a hydrophobic shielding phenomenon, where a larger concentration of hard segments is present at the exterior of the polyester urethane construct. In some embodiments, a drug release delay mechanism is tuned by concentration or arrangement of hard segments, primarily through selection of appropriate soft and hard segment chemistries.

In terms of degradation behavior, water uptake can be regulated through the chemistry of the hard/soft segments, as well as the spatial arrangement of phase-segregated polyurethane microstructures. As API release may be partially dependent on diffusion of water into the polymer matrix and the restrictive diffusivity-based and/or affinity-based interactions that hold the API within the polymer, these factors also have a significant impact on sustained release trends.

In some embodiments, the mesh size of the polyester urethane crosslinked network dictates the permeability and diffusivity of water molecules, drug molecules, and polymer degradants through the polymer matrix. For example, small molecules and peptides up to approximately 4 to 6 kDa can permeate through a PGSU matrix based on a high molecular weight PGS with 1:1 glycerol:sebacic acid stoichiometric ratio crosslinked with HDI at 1.1:1 to 1:1 isocyanate-to-hydroxyl stoichiometric ratio. However, larger molecules like proteins with 22 kDa, 66 kDa, at 150 kDa cannot permeate as easily through the PGSU matrix, likely due to a mesh size filtration effect. An increased mesh size may increase water infiltration into the polymer network and accelerate degradation. An increased mesh size may also accelerate release kinetics.

In exemplary embodiments, a micro-phase separation of soft segments and hard segments, resulting from chemical differences, such as hydrophilicity/hydrophobicity, increases the duration of drug release. Differences in the polarity of the hard segment and soft segment give rise to hydrophilic versus hydrophobic properties. For example, a discrepancy of hydrophilicity and hydrophobicity between the soft segments and the hard segments led to an increased duration of release of dexamethasone. In one case, three varieties of polyesters were synthesized and made into urethanes with either LDI or HDI (see Example 32). A polyester urethane from a low molecular weight PGS synthesized with a 1:1 glycerol:sebacic acid stoichiometric ratio combined with HDI led to an increased duration of release relative to the same low molecular weight PGS combined with LDI. Similarly, PG:Ad synthesized with a 1.1:1 mol:mol glycerol:adipic acid stoichiometric ratio and combined with HDI led to an increased duration of release relative to the same PG:Ad combined with LDI. Unexpectedly, and in contrast, a polyester urethane from a high molecular weight PGS synthesized with a 1:1 glycerol:sebacic acid stoichiometric ratio and combined with LDI led to an increased duration of release relative to the same high molecular weight PGS combined with HDI, demonstrating an opposite trend. These polyester urethanes were formulated with either LDI or HDI at equivalent isocyanate-to-hydroxyl ratio in the range of 1:1 to 1.1:1. In all cases, LDI helped enable a faster degradation of the polyester urethane network compared to HDI, although in all other cases, the LDI also caused faster and thus a decreased duration of release of the hydrophobic drug dexamethasone.

High molecular weight PGS-LDI was an interesting situation where release of the hydrophobic drug dexamethasone was slowed down, compared to high molecular weight PGS-HDI, breaking from the trend of other observed formulations. This is useful, since it allows formulations that degrade faster but provide an increased duration of release, whereas most other formulations that degrade faster also suffer from a faster release. The improvement in sustained release for the high molecular weight PGS-LDI may be attributed to an increased degree of phase separation between the highly branched, hydrophobic PGS chains and the hydrophilic lysine-terminated LDI. The rate of crosslinking by LDI was slower than with HDI. This may provide more time for phase separation to occur between the soft and hard segments of the polymer matrix, creating larger pockets of hydrophobic PGS regions crosslinked to each other by the isocyanate.

In some embodiments, a polyester urethane is loaded with 60 wt % of an NSAID and molded into a shape such as a disk or a rod for oral, ocular, or subcutaneous delivery. In some embodiments, the molded shapes are coated with unloaded polymer such as conventional PGSU to moderate and control drug release.

In one example, low molecular weight PGS with low branching crosslinked with HDI degraded faster but released dexamethasone more slowly, relative to a conventional PGSU from PGS with high molecular weight and high branching crosslinked with HDI. Without wishing to be bound by theory, it is believed that although the shorter PGS chains had a higher frequency of ester bonds to allow faster degradation, being more linear, they had more pendant hydroxyl groups available for isocyanate attack during urethane crosslinking, forming a network with soft segments largely along the polymer backbone and hard segments largely as orthogonal branches. Keeping the ester bonds largely within the polymer backbone, in a lower branched more linear resin, may allow more water interaction with those ester bonds once formed into the polyester urethane network. Similarly, keeping the urethane bonds largely forming the branches in a lower-branched, more linear resin may allow more drug interaction with those urethane bonds once formed into the polyester urethane network.

In some embodiments, the synthesis/crosslinking reaction rates of resins synthesized with more than one polyacid are tuned to create distinct regions of PG:Ad/PG:Suc/PG:Seb. These alternating or random crystalline soft segment regions including different polyacids can impart physical and chemical properties that can be used to tune mechanical properties and/or API release. For example, a PG:[25:75]Ad:Seb polyester resin (see the beginning of the Examples section for resin nomenclature) formulated to include a 60 wt % dexamethasone-loaded polyester urethane construct imparted sufficient hydrophobic crystalline regions of sebacic acid-rich soft segments that release could be sustained from these regions.

Variation of the polyacid ratios in copolymer synthesis can be used to tune the release kinetics of hydrophobic APIs. Similarly to phenomena observed with degradation rates, the use of a PG:[50:50]Ad:Seb polyester resin in a polyester urethane did not result in a release curve that fell exactly in between that of PG:Seb and PG:Ad alone for every API.

For example, a polymer of glycerol, polyacid, and diisocyanate was loaded at 60 wt % with an API that is a non-steroidal anti-inflammatory drug (NSAID). The hydrophobic NSAID drug had a solubility of about 50 g/mL in PBS and was encapsulated within a polymer matrix of PGAdU. A more rapid drug release profile was observed, with greater than 40% of drug payload released within the first two weeks, compared to conventional PGSU. When the same hydrophobic drug was encapsulated within a polymer matrix of PG:[50:50]Ad:Seb-U, however, a slower release profile was obtained, with only between 10-20% of the drug being released in first two weeks, which was much closer to the release profile observed when the drug was released from a polymer matrix of conventional PGSU, where it released approximately 10% of the API payload in the first two weeks. In another embodiment, however, where the same polymer materials were loaded with the hydrophobic drug dexamethasone, having a solubility of about 75 μg/mL in PBS, the release of the API from PG:[50:50]Ad:Seb-U was much closer to that of its release profile from PG:Ad-U rather than PGSU.

The microstructural arrangement of the PG:Suc, PG:Ad, and/or PG:Seb soft segments not only impacts the frequency of ester bonds and therefore the rate of hydrolysis, but also the crystalline behavior of each segment. If soft segments are combined as block copolymers, the overall polyester urethane may exhibit physical, chemical, mechanical, degradation, and release properties as two separate materials. If the copolymer combinations are synthesized with alternating chains of PG:Suc, PG:Ad, and/or PG:Seb, the neighboring polyacid segments may exhibit unexpected effects on the degradation and release kinetics. For example, contrary to what has been reported in literature for copolymers of polyethylene oxide (PEO) and polycaprolactone (PCL), where incorporation of PEO blocks into PCL-PEO-PCL triblock copolymers or decreasing the length of the PCL segment has been shown to increase hydrolysis and decrease crystallinity, polyester resin microstructure could instead arrange in a way that accelerates hydrolysis while having no effect on or even increasing crystallinity. For example, certain crosslinkers may miscibilize better in certain regions of the polyester resin, leading to an accelerated crosslinking reaction rate.

Different polyols and polyacids can be chosen for increased polarity, hydrophilicity, and consequently increased interaction with water for hydrolysis. Besides the chemical composition of the building blocks, the architecture can also be chosen for increased water access for hydrolysis. The architecture may include, but is not limited to, the degree of branching, the spacing between branch points, the length of the branches, the spacing between ester bonds, and/or the spacing between urethane bonds, among other features.

Partitioning can occur due to a number of factors, including, but not limited to, drug miscibility in the soft segment, drug miscibility in the hard segment, drug solubility in aqueous media, the partition coefficient of between octanol and water (log(P)) of the drug, mobility of the drug, permeability of the drug in the polymer phase, diffusivity of the drug in the polymer phase, affinity of the drug to the soft segment, affinity of the drug to the hard segment, non-covalent interactions with the soft segment, non-covalent interactions with the hard segment, drug dispersion in the soft segment, drug dispersion in the hard segment, drug-drug interactions, temperature during formulation processing, melting, crystallization, and glass transition temperatures of the drug, and/or solubility of the drug in any solvent system used during formulation processing.

Given the potential for partitioning, the order of addition of ingredients during the mixing step is therefore important. The drug may be added directly to the polymer first, the drug may be added directly to the isocyanate first, or the drug may be added to a pre-mixed polymer-isocyanate blend. In a solvated system, the drug may be added to the solvated system first. In a solvated system, the drug may be dissolved in a solvent and then mixed with polyester resin, isocyanate, or pre-mixed polymer-isocyanate blend. The appropriate conditions may help to stabilize the drug in the amorphous state in the PGSU matrix. The presence of solvent may affect the partitioning of the drug and spatial arrangement of the polymer.

In some embodiments, the drug is added to the polymer first, then the isocyanate is added in a second mixing step. In some embodiments, it may be advantageous to allow equilibration between mixing steps, to afford time for any arrangement or rearrangement to occur. In cases where multiple types of polymers are blended together, the order of addition again is important. In some embodiments, it may be advantageous to first mix a hydrophobic drug with a hydrophobic sebacic acid-containing polymer first, then add a hydrophilic polymer into the mix second. This may allow favorable interactions between the hydrophobic polymer and hydrophobic drug, providing a good affinity between the two, good drug dispersion, and good drug entrapment, prior to adding other formulation components. Step-wise addition of drug, resin, or isocyanate may be necessary. In some embodiments, step-wise addition of drugs in multiple mixing steps best incorporates and disperses the drug.

For these mixing considerations, wetting of the drug particles by the formulation components is important for good dispersion and a good interface between the drug particle and the crosslinked polymer network without any air gaps and/or porosity. Both drug-polymer dispersion and drug-polymer interface impact the release kinetics. The drug release kinetics in turn impact polymer degradation rate, due to the porosity left behind by the drug particle. Drug particle size and shape, drug dispersion, or alternatively drug aggregation, has a significant impact on drug release kinetics and polymer degradation kinetics (see, for example, Examples 28 and 29). The surface tension and surface energy of all ingredients should also be considered, as well as the attractive and repulsive forces between ingredients. Various polyester urethane compositions achieve different dispersion and wetting behaviors of the API.

Various polyester urethane compositions achieve different rheological behaviors. The viscosity and rheology of the resin is another important factor driving drug particle distribution and wetting, as well as homogeneous mixing of all ingredients. For example, adipic acid-containing resins and succinic acid-containing resins demonstrated higher viscosity than sebacic acid-containing resins, with succinic acid compositions exhibiting the highest viscosity of all, almost to the point of being unflowable at formulation temperatures in the range of 25° C. to 45° C. This makes adipic acid being a preferred polyacid over succinic acid, among other impacts on degradation. Molecular weight is a common tunable factor linked to viscosity, although it is not always predictable how a branched polymer's viscosity trends with its molecular weight. For example, low molecular weight PGS exhibited a lower viscosity than high molecular PGS, and it was correspondingly easier to mix, formulate, flow, and dispense or to extrude or mold. In another example, low molecular weight PGS synthesized with a 1:1 glycerol:sebacic acid stoichiometric ratio had a similar M_{w} of about 6,000 Da to low molecular weight PGS synthesized with a 1.1:1 glycerol:sebacic acid stoichiometric ratio, the former having high branching of about 15 mol % 1,2,3-triacylglyceride while the latter having low branching of about 11 mol %, the former having a viscosity of about 1.5 Pa-s while the latter having a viscosity of about 1 Pa-s. This comparison highlights that molecular weight cannot unilaterally predict viscosity, but rather branching plays a role as well.

In some embodiments, all formulation components are added step-wise and mixed using high-shear speed mixing. In some embodiments, the speed mixing generates heat. If the temperature of the formulation mixture is above the glass transition temperature of the drug, the mobility of the drug in the amorphous state may be increased. This may lead to increased partitioning in the soft or hard segments, depending on the affinity of the drug to the polymer components.

Since temperature impacts the viscosity and polymer chain mobility of these polyester resins, the temperature during formulation is important to control. The temperature of the resin has a different impact on the orientation of linear polymer chains compared to branched polymer chains. The temperature also affects the mobility of the hard and soft segments of the polymer. This may lead to preferential partitioning of the drug in either segment. In some embodiments, a predetermined temperature is selected to elicit soft segment aggregation and condensing, whereas another temperature may allow the soft segment to intermingle more with other formulation components. Various polyester urethane compositions achieve different miscibility behaviors of the resin and the diisocyanate. This may be due to viscosity differences between the resin and the isocyanate, or it may be due to solubility, polarity, or surface energy differences. For example, diisocyanates did not incorporate and miscibilize as easily into adipic acid-containing resins or succinic acid-containing resins, even under high shear mixing, compared to sebacic acid-containing resins.

In some embodiments, solvating the resin has an impact on the viscosity and thus the drug dispersion. For example, making the soft segment into a solution using acetone and/or propyl acetate at 40 to 60 wt % polymer content reduced the viscosity significantly. Solvated formulations were easier to process, mix, flow, and extrude. In a particular example, P[1:1]G:[50:50]Ad:Seb(8 k)-HDI (14-HDI, see Table 1 for resin abbreviations), made with 1:1 mol:mol G:Ad-S and 50-50 mol % Ad-S, was solvated for formulation. The resulting solvated drug-loaded implants demonstrated sustained release with one particular hydrophobic drug (a non-steroidal anti-inflammatory drug (NSAID) with a log(P) of 1.9 and 50 μg/mL solubility in phosphate-buffered saline (PBS)), with longer duration release compared to a different hydrophobic drug (a corticosteroid with a log(P) of 1.93 and 75 μg/mL solubility in PBS) loaded into a solvent-free version of the same formulation. In both formulations, neither drug exhibited solubility in any formulation component, including polyester resin, isocyanate, and if present, solvent. Unexpectedly, in this case, the solvated formulation released drug more slowly than the solvent-free version, which was opposite of a previously observed trend. Without wishing to be bound by theory, it is believed that solvated formulations have a larger mesh size in the crosslinked network, due to polymer swelling and spacing during crosslinking, which is then followed by solvent evaporation. This typically allows greater drug and water permeability and diffusivity through the polymer network leading to faster release kinetics compared to solvent-free formulations. There is some evidence that solvated formulations degrade faster than solvent-free formulations. There is also evidence that solvated formulations of PGSU release drug faster than solvent-free PGSU, for a wide range of drugs. The favorable extended release kinetics observed with solvated 14-HDI, with 1:1 mol:mol G:Ad-S and 50-50 mol % Ad-S, may have been due to better drug wetting and dispersion arising from the lower viscosity. Another possibility is that the presence of solvent causes a unique interaction between the soft segment and hard segment, causing greater drug partitioning into a given phase, or causing greater phase separation. The presence of solvent may orient soft and/or hard segments of the crosslinked polymer in a way that lowers water infiltration. Alternatively, the increased mesh size left behind due to solvent evaporation may increase tortuosity and hence, surface area available for the drug to interact as it is released. Another possibility is that two different hydrophobic drugs can exhibit different release kinetics from the same soft segment and hard segment compositions, due to any differences in partition coefficient, solubility, polarity, particle size, particle morphology, and interactions with the hard and soft segments.

In support of the notion that different hydrophobic drugs can exhibit different release kinetics from the same soft segment, it was observed that in the release of two different hydrophobic APIs into PBS trended differently when embedded within polymer matrices having different ratios of adipic acid and sebacic acid content. The two hydrophobic APIs were again dexamethasone, having a log(P) of 1.93 and solubility of about 75 μg/mL in PBS, and an NSAID having a log(P) of 1.9 and solubility of 50 μg/mL in PBS. Both drugs released more slowly in polymer matrices where the polyester resin was composed of 1-HDI and much more quickly from polymer matrices where the polyester resin was composed of 7-HDI. However, in matrices where the polyester resin was composed of 14-HDI, where the ratio of adipic acid to sebacic acid was 1:1, dexamethasone released relatively quickly with about 63% of payload being released in two weeks, more similarly to the 7-HDI formulation with about 67% being released in two weeks, as opposed to the 1-HDI with only about 22% being released in two weeks. Conversely, the hydrophobic NSAID released much more slowly from the 14-HDI, with 10-20% release of the drug payload in the first two weeks, than it did from the 7-HDI matrix, with greater than 40% drug release in the first two weeks, almost as slowly as the 1-HDI formulation, with about 10% drug release in the first two weeks. These findings demonstrate that predicting the release kinetics of a given API from a range of soft segments is not as straightforward as simply comparing the hydrophobic to hydrophilic monomer content within the polyester resin.

In some embodiments, the spatial arrangement of soft and hard segments contributes to altering water infiltration and therefore rate of hydrolytic degradation. It is possible to use phase separation to create “shielding” of soft segments based on intermolecular interactions between segmented soft and hard domains. The hard segment domains may be preferentially oriented toward the surface, perhaps as a result of molding, intermolecular interactions between the segments, or thermodynamic phenomena. When exposed to aqueous media, the hard segments may act as a shield at the water-exposed surface, while the soft segments remain in the interior and less accessible by water. This may lead to a slower degradation or shielding of hydrolysis, consequently, when the hard segments are more crystalline, and the soft segments are more amorphous.

In some embodiments, an increased degree of phase separation results in an increased mobility of soft segments, resulting in shifting of the soft segments to more favorable thermodynamic states by minimizing interfacial free energy. The soft segment itself can experience phase separation or self-aggregation as well, where the hydrophilic glycerol-rich hydroxyl-containing domains of the polymer network spatially coordinate, and the hydrophobic sebacic acid-rich carboxyl-containing and aliphatic chain-containing domains of the polymer network coordinate. In this case, the preferential orientation of hydroxyl groups towards the exterior of the soft segment domain results due to interaction with water, while the carboxylic acid groups aggregate interior to the domain and interact with entrapped drug particles. This particular spatial arrangement of soft segment domains may offer the benefit of exposing hydrolyzable chain segments to water, while protecting and retaining API and thus sustaining release. One reason why more hydrolyzable polyester resins, such as, for example, poly(glycerol adipate) (PG:Ad), may not be easily achieved after further crosslinking using urethane chemistry is that the crosslinked end product may lose its more rapid hydrolyzable properties and degradation benefits due to the stable nature of the urethane bonds. This would be a more likely expectation with higher concentrations of diisocyanate crosslinker, such as for an isocyanate-to-hydroxyl stoichiometric ratio in the range of approximately 1:1 to 1.1:1. The library of polyester urethanes described herein, however, show that the higher rates of hydrolysis seen in the pre-polymer is typically carried forward into the post-crosslinking polyester urethane state despite the presence of the “non-degradable” urethane bonds. The degradation profiles of these particular polyester-urethanes are governed by the uptake of water. The higher the ability to take up water into the matrix is, the more likely is the chance of faster hydrolysis. Additionally, the mobility of the degradants assists their permeability through and departure from the bulk network. Shorter chain polyacids like adipic acid and succinic acid, and their corresponding oligomers with polyols and isocyanates, are able to maneuver through the polymer mesh network more easily than sebacic acid and its analogous oligomers. Shorter chain polyacids like adipic and succinic acid may also be able to acid-catalyze the ester hydrolysis to a greater extent than sebacic acid, due to increased maneuverability to access and cleave multiple ester bonds.

In some embodiments, the packing of soft segment polymer chains, based on a low molecular weight of the soft segment, improves sustained drug release. In some embodiments, the low molecular weight is below about 6,500 Da weight average molecular weight and below about 2,500 Da number average molecular weight. For example, a low molecular weight soft segment unexpectedly led to improved dexamethasone sustained release.

In some embodiments, the packing of soft segment polymer chains, based on the low branching of the soft segment, improved sustained drug release. As previously mentioned, low branching refers to below approximately 15% mol 1,2,3-triacylglyceride or below 0.36 degree of branching (DOB) as calculated by DOB=2D/(2D+L), where D represents the mol % proportion of 1,2,3-triacylglyceride, and L represents the combined mol % proportions of 1,2-diacylglyceride and 1,3-diacylglyceride. For example, low branching of the soft segments unexpectedly led to improved dexamethasone sustained release. The soft segments with lower molecular weights had a lower mol % of 1,2,3-triacyleglyceride portions.

In some embodiments, the packing of the soft segment polymer chains, based on the long polyacid component of the soft segments, improves sustained drug release. The higher molecular weight polymers of the long polyacid component here has a carbon chain length of eight or greater and showed better sustained release compared to shorter polyacids. Without wishing to be bound by theory, this may be an effect of the higher branching nature of the polymer chains and the denser collapsed spatial arrangement of the chains. This would enhance the polymer-API hydrophobic interactions and physical entrapment of the API. In addition, this hydrophobicity of the polymer matrix also impacts the infiltration of water molecules into the matrix, which assists in providing a better sustained release of the API. For example, use of sebacic acid with eight carbons and a linear structure improved dexamethasone sustained release over adipic acid or succinic acid with four and two carbons, respectively, also with linear structures. Additionally, as the number of carbons within the polyacid component increases, the polarity and hydrophilicity of the polymer chain decreases. As a result, interactions between hard and soft segments are reduced while interdomain interactions within the soft segment are increased, resulting in more interaction with entrapped hydrophobic API particles.

In some embodiments, the packing of the hard segment polymer chains, based on a long, linear diisocyanate component of the hard segments, improved sustained drug release. A long diisocyanate component here is a carbon chain six or greater in length. For example, the use of HDI with six carbons and a linear structure improved dexamethasone sustained release over LDI with five carbons and a pendant moiety branching off. In one surprising and contrasting example, use of LDI, when the soft segment was purely PGS of any molecular weight or branching, improved dexamethasone sustained release over HDI, perhaps highlighting that the hydrophilic/hydrophobic disparity may be an important mechanism promoting the formation of more packed, crystalline soft segment regions.

In some embodiments, the packing of the hard segment polymer chains, based on a long, linear diisocyanate component of the hard segments, also impacts degradation. For example, the use of 12DI with twelve carbons and a linear structure slowed down degradation even more than HDI with six carbons and a linear structure. LDI with five carbons and a pendant moiety exhibited the fastest degradation of all three. Once again, the increased degree of interdomain interactions within the hard segment and the greater phase separation produced an increased crystallinity that slowed the diffusion of water into the polymer matrix.

In some embodiments, the hydrophobicity of the soft segments, based on sebacic acid, improved the sustained drug release of a hydrophobic drug. For example, the use of hydrophobic sebacic acid improved dexamethasone sustained release over hydrophilic adipic acid or hydrophilic succinic acid. The molar ratio of 0.9:1 glycerol:sebacic acid may be a useful soft segment composition, due to greater sebacic acid content, and hence greater hydrophobicity and less polarity.

In some embodiments, the hydrophobicity of the soft segments, based on glycerol, adipic acid, and/or succinic acid, hastens the degradation rate. For example, the use of hydrophilic adipic acid or succinic acid accelerated degradation speed over hydrophobic sebacic acid. Sebacic acid is considered more hydrophobic than adipic acid or succinic acid due to its longer aliphatic chain. It was unexpectedly observed that poly(glycerol adipate) (PGAd), even with a very high crosslink density, degrades significantly faster than PGS. This effect has been seen with thermoset resins using short chain acids where permeability of the tetrahydrofuran (THF) is low (see Godinho et al.), but the effect seen with the crosslinked version where the resin thermoset properties are mimicked is unexpected. Without wishing to be bound by theory, it is believed that the rate of degradation is governed by the number of ester linkages in the polyester over the urethane crosslinks in the matrix, going from the longer diacid (sebacic acid) to the shorter diacid (adipic succinic acid). As the ester bonds make the system more hydrophilic, the rate of degradation is increased. The chain packing of the resin with the assistance of crosslinking creates a tighter network, thus causing the reduction of the permeability of THF. However, in the presence of water, this effect is diminished possibly due to the increased rate of hydrolysis and permeation of water due to the increased hydrophilicity of the matrix.

In some embodiments, the hydrophilicity of the hard segments, based on LDI, hastens the degradation rate. For example, the use of hydrophilic LDI accelerated the degradation rate over hydrophobic HDI. HDI is considered more hydrophobic than LDI due to its slightly longer aliphatic chain, and LDI is also more hydrophilic due to the ester present in its pendant group.

In one example, 50 mol % of the sebacic acid in the polyester resin was replaced with adipic acid, and the resulting resin was crosslinked with HDI, resulting in degradants that were over twice as large as conventional PGSU degradants. In contrast, switching from adipic acid to succinic acid, an even shorter polyacid, led to only a marginal increase in degradant solubility, around 30%, which was within statistical error of the adipic acid. This points towards a unique range of polyacid length that results in favorable tunable benefits. Specifically and unexpectedly, partially replacing an eight-carbon polyacid with a four-carbon polyacid led to a significant increase in degradant solubility that was not statistically different than the effect observed when replacing the eight-carbon polyacid with a two-carbon polyacid. Polyester urethane polymers made with 50 mol % adipic acid substituted pre-polymer degraded and fully solubilized five times faster than conventional PGSU made with the same crosslinking density. It was observed that succinic acid incorporates at a slower rate and does not incorporate as completely as adipic acid and sebacic acid do during a water-mediated polycondensation reaction with glycerol. Consequently, the chain organization of glycerol adipate-sebacate (GAd:Seb) is likely quite different than the organization of glycerol succinate-sebacate (GSuc:Seb), although difficult to determine or quantify. Relatedly, the biodegradant structure, their repeat unit pattern, and their solubility are different between GAd:Seb and GSuc:Seb. Sebacic acid solubility in water is 0.25 mg/mL, compared to adipic acid solubility of 24 mg/mL and succinic acid solubility of 58 mg/mL. Incorporating shorter diacids yields polyester urethane formulations that produce more soluble degradant monomers and degradant oligomers and increases the average water solubility of the polyacid component. For example, [25:75]Ad:Seb has an average solubility of 6.19 mg/mL, [50:50]Ad:Seb has an average solubility of 12.13 mg/mL, and [50:50]Suc:Seb has an average solubility of 29.13 mg/mL.

In some examples, the resulting polymers including copolymers of two different polyacids displayed degradation kinetics that closely match that of the polymer of the shorter polyacid but displayed softer mechanical properties and better drug release profiles more like the polymers of the longer polyacid than the polymer of the shorter polyacid. In some examples, 95:5, 80:20, 75:25, 50:50, 25:75, 20:80, or 5:95 mol % succinic-sebacic acid (Suc:Seb) or adipic-sebacic acid (Ad:Seb) copolymers behaved more like one acid or the other, instead of like an average or expected weighted average of the two, due to the architecture of the polymer being more of a gradient copolymer and less of a random polymer (see, for reference, Alam et al., “Gradient copolymers—Preparation, properties and practice”, European Polymer Journal, Vol. 116, pp. 394-414, (2019)). By this approach, degradable polyester urethanes that match some of the shelf stability, low extractables and leachables content, high crosslink density, and low mesh size of conventional PGSU surprisingly achieved two to seven times faster degradation rates. In some embodiments, PG:Ad, using 100% adipic acid, provides faster degradation. In some embodiments, PG:[50:50]Ad:Seb and PG:[50:50]Suc:Seb, using 50:50 mol:mol adipic acid:sebacic acid and 50:50 mol:mol succinic acid:sebacic acid, respectively, provide for faster degradation and sustained release of some drugs. In some embodiments, PG:[25:75]Ad-Seb, using 25:75 mol:mol adipic acid:sebacic acid, provides sustained release while still offering faster degradation. In some embodiments, PG:[95:5]Suc:Seb has a very high viscosity that is on the border of workability, though these formulations may degrade the fastest of all, proving the contribution of shorter diacids and greater ester bond frequency for enhanced hydrolysis.

In fact, some of the biodegradable polyester urethanes made of a soft segment composed partially or fully of a polyacid shorter than sebacic acid appeared to be two to four times more crosslinked than conventional PGSU, based on the test results from Flory-Rehner solvent swelling, when they are reacted with the same amount of diisocyanate at the same isocyanate-to-hydroxyl stoichiometric ratio. Surprisingly, however, they degraded between two and seven times faster than conventional PGSU, highlighting the unexpected effect of soft segment hydrolysis on a polymer that is composed of a 20% non-degradable hard segment.

Additionally, pre-polymers with two different polyacids, one short and one long, that were further made into a polyester urethane might be expected to exhibit hydrolysis and degradation profiles that are a linear combination of the expected degradation properties of the individual polyacids alone. In some cases, however, it was found that these polyester urethanes exhibited hydrolysis and degradation profiles that very closely resemble that of the shorter polyacid alone and not the longer polyacid. Thus, they had faster degradation times than the expected average. The presence of the longer polyacid, however, led to lower viscosity resins that were more workable, more favorable softer mechanical properties of the end polyester urethane polymer, and regions of hydrophobicity within the network that enhanced sustained drug release of hydrophobic drugs, relative to polymers made with only the shorter polyacid.

Additionally, pre-polymers including one short and one long polyacid resulted in copolymers with a lower degree of branching than the equivalent molecular weight polymers including the shorter polyacid alone. It was observed in some cases that a soft segment with a lower degree of branching, but a similar molecular weight, resulted in polymers with more favorable sustained dexamethasone release profiles when crosslinked with a diisocyanate. The molar percentage of 1,2,3 triacylglyceride, a measure of branching determined by 13C-NMR, for PG:Ad(8 k) with a weight average molecular weight of about 8,000 Da was 14.1 mol % while PG:[50:50]Ad-Seb(8 k), the copolymer of equimolar sebacic acid and adipic acid of a similar molecular weight, had a lower 1,2,3 triacylglyceride molar percentage of 13.1 mol % (see Example 4).

In some embodiments, two or more polyester resins having different polyacid compositions are blended together as the polyester resin for crosslinking with a diisocyanate to increase the hydrolysis rate of the resulting polyester urethane relative to conventional PGSU. In some embodiments, the physical blending achieves an arrangement of various polyacid regions in the resulting polyester urethane.

In some cases, significantly more viscous, but also more hydrolyzable, pre-polymers that have intrinsic processability issues were blended with less viscous prepolymers, creating a homogenous pre-polymer blend that was more processable and was then successfully crosslinked using diisocyanates to form a degradable polyester urethane with more hydrolyzable network components. In some examples, the resulting polymers had two distinct soft segment components that could provide a wider range of physiochemical and mechanical properties. The polyester resins needed to have a certain degree of miscibility in each other. PG:[95:5]Suc:Seb and PG:Seb separated into two distinct layers if left at room temperature for 10 minutes, and when speed-mixed and crosslinked to form a polyester urethane, the resulting polymer showed a two-phase degradation that resulted in polymer cracking and unpredictable degradation profiles. On the other hand, PG:[95:5]Ad:Seb mixed with PG:S to a clear and homogenous composition whose urethane showed faster degradation kinetics relative to conventional PGSU, resembling a more hydrolyzable network and following the degradation profile of a homogeneous network. Additionally, a copolymer of adipic acid and sebacic acid instead of poly(glycerol adipate) directly in a mixture achieved better mixing and homogeneity in the blend.

In some embodiments, a free acid additive included in the polyester urethane composition acts as a foaming agent through the generation of carbon dioxide gas. Synchronous generation of carbon dioxide gas and crosslinking of the polyester urethane serves to entrap the gas particles to produce a foamed material. The concentration and size of pores can be tuned by controlling the crosslinking and carbon dioxide generation. Alternatively, a physical porogen may be included within the polyester urethane formulation and leached out post-crosslinking to create a porous substrate. Foamed materials may be beneficial in applications that require rapid diffusion and payload release and can promote release and delivery of large molecules that would otherwise remain entrapped within an un-foamed crosslinked substrate. Foamed substrates also promote infiltration of cells and biological material in applications for bioresorbable implants. Additionally, porous polyurethane materials are commonly used in orthopedic applications requiring resistance to compressive stresses, enabled by the coupled viscoelastic behavior of the polyester urethane and water-filled pores.

In addition or alternatively to adjusting the length of the polyacid, the polyol can similarly be adjusted to change the frequency and spacing of ester bonds. Alternative appropriate polyols may include, but are not limited to, polyethylene glycols or polyorthoesters. Polyorthoesters can be incorporated to introduce hydrolysis sites with significantly faster degradation rates. In some embodiments, the polyol component includes a mixture of two or more polyols, such as, for example, glycerol and PEG300. Various combinations of polyols, such as, for example, 5-95 mol %, 10-90 mol %, alternatively 20-80 mol %, alternatively 25-75 mol %, alternatively 50-50 mol %, or any value, range, or sub-range therebetween, may be used.

In some cases, the interactions between some of the parameters used to modulate degradant solubility were also non-obvious and unexpected. For example, a lower molecular weight PG:Seb polyester resin having an Mw of around 6,000 Da in combination with an LDI crosslinker produced polyester urethane degradants with more than double the number average molecular weight (Mn) of the degradants of a higher molecular weight PG:Seb polyester resin having an Mw of around 15,000 Da crosslinked with LDI at the same crosslinking density (see Example 21). Peak 1 degradants of the LDI polyester urethane formed from low Mw PG:Seb as measured by gas permeation chromatography (GPC), showed an Mn of 13,186 Da compared to an Mn of 5917 Da for the Peak 1 degradants of the LDI polyester urethane formed from the higher Mw PG:Seb. The Mw of these largest degradants, however, were more similar at 19,858 Da for the low Mw PG:Seb-LDI polyester urethane compared to 17,540 Da for the higher Mw PG:Seb-LDI polyester urethane. When looking at the entire population of degradants, the low Mw PG:Seb-LDI polyester urethane yielded lower Mw degradants (10,893 Da) than the higher Mw PG:Seb-LDI polyester urethane (13,004 Da). The proportion of large degradants and small degradants was different between these two formulations. Also in this example, for both higher Mw PG:Seb and lower Mw PG:Seb, LDI crosslinking yielded degradants of these large sizes that were completely water soluble. This contrasts with the same higher Mw PG:Seb crosslinked with HDI, whose degradants had to become very small in size in order to be completely water soluble. Specifically, its Peak 1 degradants showed an Mn of 1,482 Da and an Mw of 1,670 Da, and the entire population of degradants was Mw of 545 Da.

Beyond the solubility of the degradants, the acidity of the monomers, oligomers, and degradants can help accelerate degradation, through acid-catalyzed hydrolysis of ester bonds. Succinic acid as a diprotic acid has two pKa values, which are 4.21 and 5.64 at 25° C. The two pKa values of adipic acid are pKa is 4.41 and 5.41. The strongest pKa value of sebacic acid is 4.72. A lower pKa value reflects a stronger acid and greater ability to donate a proton in aqueous solution. This may render succinic acid and adipic acid more acidic and better at acid-catalyzing hydrolysis.

The ability of degradants to leave the polymer network also influences whether the polymer is surface eroding versus bulk degrading. Even without biodegradant solubility, maneuverability, or evacuation, the polymer network still experiences some hydrolytic cleavage, but the dimensions and the mass do not reduce over time. When the polymer network degradation and its degradants are not solubility-limited, dimensional loss and mass loss can occur in a controlled way at a predictable rate. Moreover, lingering degradants can delay degradation by preventing sufficient water interaction for hydrolysis, which may be the case with lingering sebacic acid, given its non-polar, hydrophobic, and water-insoluble nature.

In addition to providing more soluble degradants, LDI has the further advantage of a much more biocompatible diisocyanate crosslinker than HDI, given that HDI converts to hexamethylene diamine (HDA), which has toxicity concerns in vivo, upon degradation whereas LDI degrades into lysine and ethanol. In general, hydrolyzing the amide bond of the urethane component yields a carbamic acid that is inherently unstable and quickly reacts with water to form an amine and carbon dioxide, which are excreted and expired, respectively. When HDI is the crosslinker in PGSU, the HDA that results from hydrolysis can exhibit acute toxicity by oral and inhalation routes of exposure and moderate toxicity by dermal route, although HDA does not induce skin sensitization, is not a developmental or reproductive toxin, and may do only limited systemic damage, and any irritation observed is proportional to HDA exposure concentration. Notably, HDA is rapidly absorbed and metabolized in vivo with little tissue storage. HDA is partially oxidized by diamino oxidases and aldehyde dehydrogenases to 6-aminohexanoic acid, which is excreted in urine, and a small part is HDA also excreted unchanged in urine. Importantly, excretion of HDA by humans is rapid, within 10 hours. In contrast to HDA, lysine and ethanol do not have these same toxicity concerns.

Another non-obvious benefit of using LDI over other diisocyanates is the bio-friendly nature of LDI's degradants, which are lysine and ethanol. Lysine is a naturally-occurring essential amino acid that is a precursor to many proteins, which can interact favorably with the naturally-occurring metabolites sebacic acid, adipic acid, succinic acid, and/or glycerol that makes up the polymer backbone. Sebacic acid is known to reduce hyperglycemia in type 2 diabetic individuals following a meal. Lysine is also known to help with glycemic control. The combination of biodegradants from LDI-containing and sebacic acid-containing polyester urethanes is likely useful in metabolic control and diabetes.

Another non-obvious benefit of using LDI is that its degradant ethanol may assist in solubilizing the oligomers during PGSU network degradation. Ethanol is a good semi-polar solvent for glycerol, sebacic acid, adipic acid, succinic acid, oligomers containing these components, and polymers containing these components. Especially for non-water-soluble PGS and PGSU biodegradants and/or oligomers, which typically lose water solubility at sizes above 1000 Da, a small amount of ethanol resulting from LDI hydrolysis can help bring these degradants into solution. Without wishing to be bound by theory, this may be one mechanism by which LDI overcomes the water-solubility limit of PGS and PGSU biodegradants and oligomers.

Another mechanism by which LDI improves the water-solubility of PGS and PGSU species may be that LDI may allow more solvent penetration into the polymer matrix based on its hydrophilicity. Polymer dissolution occurs in two stages. First, solvent molecules diffuse through the polymer matrix to form a swollen mass known as a gel. Second, the gel breaks up and the molecules are dispersed in a true solution. In one case, LDI allows PGS and PGSU biodegradants to form a true solution in aqueous media, which occurs much earlier in time and with much large molecular weight species within the true solution, compared to HDI. LDI provides a more favorable hydrophilic environment for water molecules to diffuse in to the outer layer of the PGSU network, aiding hydrolysis. LDI also allows more swelling, which further brings in more water molecules, also aiding hydrolysis.

In some embodiments, water uptake and swelling are both important attributes influenced by the hydrophilicity of the network. Drug release and polymer hydrolytic degradation rely on access to water molecules. The water contact angle of a polymer network can be used to understand the hydrophilicity and likelihood of water interactions. It may be desirable to have a contact angle below 60 degrees, alternatively below 45 degrees, alternatively below 30 degrees, or any value, range, or sub-range therebetween, for improved water interaction. For example, the use of hydrophilic adipic acid, succinic acid, and/or LDI improved water uptake and increased aqueous swelling. If a surface erosion mechanism of hydrolytic degradation is desired, water uptake and swelling may need to be minimized. For example, the use of 100% sebacic acid as the polyacid in polyesters crosslinked with HDI or 12DI reduced water uptake and decreased aqueous swelling. For surface erosion, it may be desirable to have a contact angle above 60 degrees, alternatively above 70 degrees, alternatively above 80 degrees, or any value, range, or sub-range therebetween, for reduced water interaction. If a bulk degradation mechanism is suitable, water uptake and swelling can be maximized as a means to accelerate degradation. If a polyester urethane microstructure contains highly phase-separated soft segment and hard segment domains, water infiltration and the corresponding drug release and hydrolytic degradation may follow a different kinetic rate and different physiospatial path through the polymer microstructure compared to a less phase-separated soft segment and hard segment microstructure.

In any formulation, it is important to keep the acid value below 75 or 70 mg KOH/g, to avoid bubbles from side reactions of isocyanate with carboxylic acid off-gassing carbon dioxide during formulation. The acid value of a resin can be controlled during synthesis by having fewer pendant carboxylic acids and/or by having fewer free polyacid monomers and polyacid-containing oligomers. The unreacted monomers and oligomers can also be cleaned up and removed post-synthesis by dialysis.

The functional behavior of these crosslinked polyester urethane polymers was evaluated by looking at an array of quality attributes important for pharmaceutical drug products and medical devices, including degradation kinetics in real time conditions, degradation kinetics in accelerated conditions, drug release kinetics, drug-polymer interactions, drug-polymer spatial organization, crosslinking, water uptake, aqueous swelling, solvent swelling, sol content, extractables and leachables, and mechanical properties.

In some embodiments, polyester urethane formulations include a polyester resin, a crosslinker, and one or more optional additives, such as, for example, chain extenders, free acids, APIs, or combinations thereof, where the polyester resin soft segment is first solubilized into a solution, where acetone and/or propyl acetate may be preferred organic solvents.

In some embodiments, unloaded polyurethane formulations proceeded in a stepwise manner where the crosslinker and catalyst were added to two separate portions of polyester resin to facilitate dual-barrel extrusion, at which point crosslinking was initiated due to mixing of the polyester resin, the crosslinker, and the catalyst. The resin was first added to a speedmixer cup and mixed at 2500 RPM for 2 minutes while a vacuum was pulled on the cup, for both the catalyst (A side) and the crosslinker (B side). The catalyst and crosslinker were then added to their respective cups, along with any additives such as acid additives or chain extenders, with a pin mixer placed into each cup, and once again mixed at 2500 RPM for 2 minutes under vacuum. The respective A and B sides were then loaded into a dual-barrel syringe, which was further mixed at 2000 RPM under vacuum for final degassing. A static mixing tip was attached to the end of the dual-barrel syringe, and a hydraulic piston was used to force the material from both syringe chambers through the mix tip and into the final mold.

Although a number of isocyanates were tested, the polyester resins were all crosslinked at a crosslinking ratio in the range of approximately 1:1 to 1.1:1 isocyanate-to-hydroxyl stoichiometric ratio. Crosslinking was allowed to proceed for 24 hours, before any further sample manipulation was done.

Some unloaded compositions were molded into macro-scale shapes multiple millimeters in thickness. Unloaded compositions were tracked for mass loss throughout degradation under real time (37° C.) or accelerated (70° C.) degradation conditions, in 0.1M PBS pH 7.4 under 180-240 rpm orbital agitation.

In some embodiments, loaded polyurethane formulations proceeded in a stepwise manner where the crosslinker and the catalyst were added to two separate portions of polyester resin mixed with API to facilitate dual-barrel extrusion, at which point reactive chemistry began within the mix tip. First, the polyester resin was weighed out separately into A side and B side cups, and speedmixed at 2500 RPM for 2 minutes while a vacuum was pulled on the cup. The API was then added to each respective side, according to the proportion of resin in each cup, and speedmixed at 2500 RPM with no vacuum or pin mixer to ensure full incorporation of API into resin. The pin mixer was then added, and the resin/API blends in both the A and B sides were mixed once again at 2500 RPM under vacuum for 2 minutes. After API incorporation was confirmed, catalyst and crosslinker were added to the respective A and B sides and mixed at 2500 RPM under vacuum for 2 minutes. Both sides were loaded into a dual-barrel syringe, which was further mixed at 2000 RPM under vacuum for a final degassing. A static mixing tip was attached to the end of the dual-barrel syringe, and a hydraulic piston was used to force the material from both syringe chambers through the mix tip and into the final mold.

Some compositions were loaded with 60 wt % dexamethasone and molded into micro-scale implants having a diameter of 450 micrometers and a length of 10 millimeters. Dexamethasone-loaded compositions were tracked for mass loss and dexamethasone release throughout degradation under real time (37° C.) conditions, in 0.1M PBS pH 7.4 under 50-150 rpm orbital agitation.

In some embodiments, a PGX-based polyurethane formulation in the crosslinking range of 1:1 to 1.1:1 isocyanate-to-hydroxyl stoichiometric ratio was formed with a drug loading from 0 to 80% by weight, where the selected crosslinker, polyester resin, and additive chemistries facilitate a polyurethane microstructure that allows for matched drug release and polymer degradation by surface erosion.

In some embodiments, the microstructure, phase mixing, differential dispersion properties, and/or differential flow properties arrange zones within the 3D spatial volume of a component, such as, for example, a gradient of drug, a zone rich or poor in drug, crosslinker hard segment, or polymer soft segment, such as, for example, zones along the axial direction or zones along the radial direction for influx and travel of water and/or drug.

In some embodiments, the polyester urethane is a poly(glycerol adipate) urethane (PG:Ad-U) with glycerol for the polyol component, adipic acid for the polyacid component, and HDI for the isocyanate component (PG:Ad-HDI). This results in a more hydrophilic soft segment and a more hydrophobic hard segment.

In some embodiments, the polyester urethane is a PG:Ad-LDI with glycerol for the polyol component, adipic acid for the polyacid component, and LDI for the isocyanate component.

In some embodiments, the polyester urethane is a PG:Seb-HDI with glycerol for the polyol component, sebacic acid for the polyacid component, and HDI for the isocyanate component. This results in hydrophobic soft and hard segments.

In some embodiments, the polyester urethane is a P[1.1:1]G:Seb-HDI with glycerol for the polyol component, sebacic acid for the polyacid component, a stoichiometric ratio of polyol:polyacid of 1.1:1, and HDI for the isocyanate component.

In some embodiments, the polyester urethane is a PG:Seb-LDI with glycerol for the polyol component, sebacic acid for the polyacid component, and LDI for the isocyanate component. This results in a more hydrophobic soft segment and more hydrophilic hard segment.

In some embodiments, the polyester urethane is a P[1.1:1]G:Seb-LDI with glycerol for the polyol component, sebacic acid for the polyacid component, a stoichiometric ratio of polyol:polyacid of 1.1:1, and LDI for the isocyanate component.

In some embodiments, the polyester urethane is a poly(glycerol sebacate-co-adipate) urethane (PG:Ad:Seb-HDI) with glycerol for the polyol component, a combination of sebacic acid with adipic acid for the polyacid component, and HDI for the isocyanate component.

In some embodiments, the polyester urethane is a poly(glycerol sebacate-co-adipate) urethane (PG:Ad:Seb-LDI) with glycerol for the polyol component, a combination of sebacic acid with adipic acid for the polyacid component, and LDI for the isocyanate component.

In some embodiments, the polyester urethane is a poly(glycerol sebacate-co-succinate) urethane (PG:Suc:Seb)-HDI) with glycerol for the polyol component, a combination of sebacic acid with succinic acid for the polyacid component, and HDI for the isocyanate component.

In some embodiments, the polyester urethane is a poly(glycerol sebacate-co-succinate) urethane (PG:Suc:Seb)-LDI) with glycerol for the polyol component, a combination of sebacic acid with succinic acid for the polyacid component, and LDI for the isocyanate component. These result in a somewhat hydrophobic soft segment and more hydrophilic hard segment. The LDI helps hasten degradation and the adipic acid or succinic acid also helps hasten degradation. The sebacic acid helps sustain drug release.

In some embodiments, the polyester urethane is a P[1.1:1]G:Ad-HDI with glycerol for the polyol component, adipic acid for the polyacid component, a stoichiometric ratio of polyol:polyacid of 1.1:1, and HDI for the isocyanate component.

In some embodiments, the polyester urethane is a P[1.1:1]G:Ad-LDI with glycerol for the polyol component, adipic acid for the polyacid component, a stoichiometric ratio of polyol:polyacid of 1.1:1, and LDI for the isocyanate component.

In some embodiments, the polyester urethane is a PG:[50:50]Suc:Seb-HDI with glycerol for the polyol component, a combination of 50 mol % sebacic acid and 50 mol % succinic acid for the polyacid component, and HDI for the isocyanate component.

In some embodiments, the polyester urethane is a PG:Seb-LDI with a low M_{w} polyester resin of glycerol and sebacic acid and LDI for the isocyanate component.

In some embodiments, the polyester urethane is a PG:Seb-LDI+0.1TA with a low M_{w} polyester resin of glycerol and sebacic acid, LDI for the isocyanate component, and 0.1 wt % tartaric acid.

In some embodiments, the polyester urethane is a P[1.1:1]G:[50:50]Ad:Seb[D]-HDI with a dialyzed polyester resin of glycerol and a combination of 50 mol % sebacic acid and 50 mol % adipic acid for the polyacid component, a stoichiometric ratio of polyol:polyacid of 1.1:1, and HDI for the isocyanate component.

In some embodiments, the polyester urethane is a P[1.1:1]G:[50:50]Ad:Seb[D]-HDI with a dialyzed polyester resin [D] of glycerol and a combination of 50 mol % sebacic acid and 50 mol % adipic acid for the polyacid component, HDI for the isocyanate component, a stoichiometric ratio of polyol:polyacid of 1.1:1, and 0.1 wt % tartaric acid.

In some embodiments, the polyester urethane is a [50:50 P[1:1]G:[95:5]Ad:Seb)+P[1.1:1]G:Seb]-LDI with a polyester resin blend of 50 wt % of a first polyester resin of glycerol and a combination of 95 mol % adipic acid and 5 mol % sebacic acid at a stoichiometric ratio of polyol:polyacid of 1:1 and 50 wt % of a second polyester blend of glycerol and sebacic acid at a stoichiometric ratio of polyol:polyacid of 1.1:1, with LDI for the isocyanate component.

In some embodiments, the polyester urethane is a P[1:1]G:[25:75]Ad:Seb-HDI with a polyester resin of glycerol and a combination of 75 mol % sebacic acid and 25 mol % adipic acid for the polyacid component, a stoichiometric ratio of polyol:polyacid of 1:1, and HDI for the isocyanate component.

In some embodiments, the polyester urethane is a P[1:1]G:[25:75]Ad:Seb-HDI+0.1TA with a polyester resin of glycerol and a combination of 75 mol % sebacic acid and 25 mol % adipic acid for the polyacid component, a stoichiometric ratio of polyol:polyacid of 1.1:1, HDI for the isocyanate component, and 0.1 wt % tartaric acid.

In some embodiments, the polyester urethane is a P[1:1]G:[25:75]Ad:Seb-LDI with a polyester resin of glycerol and a combination of 75 mol % sebacic acid and 25 mol % adipic acid for the polyacid component, a stoichiometric ratio of polyol:polyacid of 1:1, and LDI for the isocyanate component.

In some embodiments, the polyester urethane is a P[1.1:1]G:Seb-HDI+0.1TA with glycerol for the polyol component, sebacic acid for the polyacid component, a stoichiometric ratio of polyol:polyacid of 1.1:1, HDI for the isocyanate component, and 0.1 wt % tartaric acid.

In some embodiments, the polyester urethane is a PG:Seb-HDI with a low M_{w} polyester resin of glycerol and sebacic acid and HDI for the isocyanate component.

In some embodiments, the polyester urethane is a P[1:1]G:[50:50]Ad:Seb-HDI with glycerol for the polyol component, a combination of 50 mol % sebacic acid and 50 mol % adipic acid for the polyacid component, a stoichiometric ratio of polyol:polyacid of 1:1, and HDI for the isocyanate component.

In some embodiments, a polyester urethane is formed from a polyester resin, having an acid value less than 75 and a hydroxyl value less than 240, crosslinked by a diisocyanate.

In some embodiments, a polyester urethane is formed from a polyester resin, having an acid value less than 60 and a hydroxyl value less than 220, crosslinked by a diisocyanate.

In some embodiments, a polyester urethane is formed from a polyester resin, having an acid value greater than 35 and a hydroxyl value greater than 180, crosslinked by a diisocyanate.

In some embodiments, a polyester urethane is formed from a polyester resin, having a zero-shear viscosity less than 5 Pas and formulated at 60° C. or below, crosslinked by a diisocyanate. In some embodiments, a polyester urethane is formed from a polyester resin, having a zero-shear viscosity less than 3.5 Pas and formulated at 30° C. or below, crosslinked by a diisocyanate. In some embodiments, a polyester urethane is formed from a polyester resin, having a zero-shear viscosity less than 2 Pas and formulated at 30° C. or below, crosslinked by a diisocyanate, and solvent-free with up to an 80% w/w drug loading.

In some embodiments, a polyester resin includes glycerol as the polyol and sebacic acid, adipic acid, or succinic acid as the polyacid.

In some embodiments, a polyester resin includes glycerol as the polyol and a combination of sebacic acid with adipic acid or succinic acid as the polyacid, with a polyacid mol:mol ratio in the range of 95:5 through 5:95.

In some embodiments, a polyester resin includes glycerol as the polyol and a combination of sebacic acid and adipic acid as the polyol, where both polyols incorporate evenly during synthesis.

In some embodiments, a polyester resin includes a polyol:polyacid 1.1:1 stoichiometric ratio.

In some embodiments, a polyester resin includes a polyol:polyacid 0.9:1 mol:mol ratio.

In some embodiments, a polyester resin includes a PGS polyester resin crosslinked by LDI. In some embodiments, the PGS polyester resin is a low M_{w} PGS.

In some embodiments, a polyester resin includes a PGS polyester resin crosslinked by LDI. In some embodiments, the PGS polyester resin is a low M_{w} PGS.

In some embodiments, the low M_{w} PGS polyester resin has a M_{w} less than 6,500 Da, a Mₙ less than 2,000 Da, and a polydispersity less than 5. In some embodiments, the low M_{w} PGS polyester resin has a M_{w} less than 3,000 Da. In some embodiments, the low M_{w} PGS polyester resin has a polydispersity less than 2.

In some embodiments, a polyester resin includes glycerol as the polyol and adipic acid as the polyol, has a low to medium molecular weight, and has a flowable viscosity at room temperature, around 20-25° C., or less than 40° C.

In some embodiments, a polyester urethane is formed from a polyester resin, with glycerol as the polyol and adipic acid as the polyacid, crosslinked by HDI. In some embodiments, the polyol:polyacid stoichiometric ratio is 1.1:1.

In some embodiments, a polyester urethane is formed from a polyester resin, with glycerol as the polyol and adipic acid as the polyacid, crosslinked by LDI.

In some embodiments, a polyester resin includes glycerol as the polyol and a combination of sebacic acid and adipic acid at 50:50 mol % as the polyacid.

In some embodiments, a polyester resin includes glycerol as the polyol and a combination of sebacic acid and adipic acid at 25:75 mol % as the polyacid.

In some embodiments, a polyester resin includes glycerol as the polyol and a combination of sebacic acid and adipic acid at 75:25 mol % as the polyacid.

In some embodiments, a polyester resin includes glycerol as the polyol and a combination of sebacic acid and succinic acid at 50:50 mol % as the polyacid.

In some embodiments, a polyester urethane includes up to 10 w/w % of a chain extender included in the blending step during formulation.

In some embodiments, a polyester urethane includes a chain extender incorporated with the isocyanate prior to the blending step during formulation.

In some embodiments, a polyester urethane is formed from an isocyanate and a polyester resin having an intentional viscosity mismatch.

In some embodiments, a polyester urethane is formed from an isocyanate and a polyester resin having an intentional miscibility mismatch.

In some embodiments, a polyester urethane is formed from an isocyanate and a polyester resin having an intentional hydrophilic-hydrophobic or polarity mismatch.

In some embodiments, a polyester urethane is formed from an isocyanate and a polyester resin in the presence of an API having intentional API particle aggregation.

In some embodiments, a polyester urethane is formed from an isocyanate and a polyester resin in a solvent in the presence of an API insoluble in the solvent to drive API partitioning.

In some embodiments, an order of addition of resin, solvent, API, and isocyanate is selected to obtain a predetermined microstructure of soft segments and hard segments.

In some embodiments, a polyester urethane is formed from an isocyanate and a polyester resin, where the isocyanate has a partition coefficient log(P) of less than 0.8.

In some embodiments, a polyester urethane is formed from an isocyanate and a polyester resin, where the isocyanate has a partition coefficient log(P) of equal to or greater than 0.8.

In some embodiments, a polyester urethane is formed from an isocyanate and a polyester resin in the presence of an API, where the API has a partition coefficient log(P) of greater than 1.

In some embodiments, a polyester urethane is formed from an isocyanate and a polyester resin in the presence of an API, where the API has a partition coefficient log(P) of equal to or less than 1.

In some embodiments, a polyester urethane is formed from an isocyanate and a polyester resin in the presence of an API, where the API has an aqueous solubility less than 100 μg/mL.

In some embodiments, a polyester urethane is formed from an isocyanate and a polyester resin in the presence of an API, where the API has an aqueous solubility equal to or greater than 100 μg/mL.

In some embodiments, a polyester urethane is formed from an isocyanate and a polyester resin in the presence of an API, where the diacid component of the polyester has an average aqueous solubility greater than 0.25 mg/mL.

In some embodiments, a polyester urethane is formed from an isocyanate and a polyester resin in the presence of an API, where the diacid component of the polyester has an average aqueous solubility equal to or less than 0.25 mg/mL.

In some embodiments, a polyester urethane is formed from an isocyanate and a polyester resin in the presence of an API, where the API has a D50 particle size of less than 10 μm.

In some embodiments, a polyester urethane is formed from an isocyanate and a polyester resin in the presence of an API, where the API is hydrophobic.

In some embodiments, a polyester urethane is formed from an isocyanate and a polyester resin in the presence of an API, where the API is a corticosteroid, a steroid, or a hormone.

In some embodiments, a polyester urethane is formed from an isocyanate and a polyester resin in the presence of an API, where the API is an NSAID.

In some embodiments, a polyester urethane is formed from an isocyanate and a polyester resin, where the isocyanate is hydrophilic.

In some embodiments, a polyester urethane is formed from an isocyanate and a polyester resin of a polyol and a polyacid, where the polyacid is hydrophilic.

In some embodiments, a polyester urethane is formed from an isocyanate and a polyester resin of a polyol and a polyacid, where the polyacid is hydrophobic.

In some embodiments, a polyester urethane with an aqueous swelling less than 10 w/w % is formed from an isocyanate and a polyester resin.

In some embodiments, a polyester urethane with sol content less than 3 w/w % is formed from an isocyanate and a polyester resin.

In some embodiments, a polyester urethane is formed from an isocyanate and a polyester resin of a polyol and a polyacid, where the polyacid includes a long hydrocarbon backbone.

In some embodiments, a polyester urethane is formed from an isocyanate and a polyester resin, where the isocyanate includes a long hydrocarbon backbone.

In some embodiments, a polyester urethane is formed from an isocyanate and a polyester resin, where the polyester resin has a radius of gyration in the range of 9 to 35 nm.

In some embodiments, a polyester resin of a polyol and a polyacid has less than 15 mol % 1,2,3-triacylglyceride.

In some embodiments, a polyester resin of a polyol and a polyacid has a more linear polyester backbone with unreacted secondary hydroxyl for later crosslinking by isocyanate.

In some embodiments, a polyester urethane has more frequent isocyanate crosslinked amide bonds along the polymer backbone relative to the frequency imparted by sebacic acid to PGSU.

In some embodiments, a polyester urethane is formed from an isocyanate and a polyester resin having a hydroxyl value greater than 180 for hydroxyl-API interactions.

In some embodiments, a polyester urethane is formed from an isocyanate and a polyester resin having an acid value greater than 30 for carboxylic acid-API interactions.

In some embodiments, a polyester urethane is formed from an isocyanate and a polyester resin, where the isocyanate includes monofunctional isocyanate coupled with difunctional isocyanate.

In some embodiments, a polyester urethane is formed from an isocyanate and a polyester resin, where the isocyanate includes monofunctional isocyanate coupled with difunctional isocyanate where the monofunctional isocyanate contains pendant functionality for API interactions.

Although described primarily for applications in drug delivery for sustained drug release, the polyester urethanes disclosed herein may have microstructures selected by the same principles described for other purposes as well.

In some embodiments, the polyester urethane bioresorbable elastomers described herein are used in biomedical engineering applications that require a bioresorbable implant or implant component. In some embodiments, the polyester urethane bioresorbable elastomers described herein are used as a polymer network for controlled release of drugs and/or biologics or as a structural medical device or a medical device component.

In some embodiments, the microstructures of polyester urethanes described herein are selected for compounding with fillers or additives other than drugs, where the same principles described herein may be applied to compounding such polyester urethane compositions.

In some embodiments, a polyester resin combining one or multiple polyacids can be used as an in situ gel with no urethane crosslinks. In some embodiments, a polyester resin is modified to incorporate reversible crosslinks.

In some embodiments, a polyester urethane bioresorbable elastomer as described herein is used in a biomedical application as a bioresorbable implant or implant component. In some embodiments, a polyester urethane bioresorbable elastomer as described herein is used as a bioresorbable coating, film, fiber, textile, microspheres, nanospheres, microneedles, or scaffold. In some embodiments, a polyester urethane bioresorbable elastomer as described herein is used as a polymer network for controlled release of drugs and/or biologics or as a structural medical device or a medical device component.

In some embodiments, the tunable microstructures of polyester urethanes described herein are selected for use in non-pharmaceutical applications, such as, for example, medical devices and cell therapy, where no drug may be present. In some embodiments, a polyester urethane bioresorbable elastomer as described herein is selected using a variety of polyacids and/or tuning the synthesis of the copolymer to actually catalyze side reactions that produce carbon dioxide, thereby producing a polyester urethane foam useful in applications such as cellular scaffolds.

In some embodiments, the microstructures of polyester urethanes described herein are selected to improve fiber properties, for fiber wet spinning, melt spinning, extrusion, electrospinning, and other processes.

In some embodiments, the controlled microstructures of polyester urethanes described herein are selected for use in film techniques, coating techniques, fiber techniques, additive manufacturing, chemical conjugation/functionalization schemes, and/or cell culture.

In some embodiments, the microstructures of polyester urethanes described herein are selected for use in microparticle formulations and nanoparticle formulations.

In some embodiments, the microstructures of polyester urethanes described herein are selected for use in in situ injectable gel drug depots.

In some embodiments, the microstructures of polyester urethanes described herein are selected for use in oral or injectable sustained release or immediate release formulations.

In some embodiments, the microstructures of polyester urethanes described herein are selected for use in targeted release, such as, for example, to target cancer cells with PGX-Us that degrade and release by acid catalysis or to target fatty cells like adipose tissue with hydrophobic PGX-Us for targeted release.

In some embodiments, the microstructures of polyester urethanes described herein are selected for use in forming larger physical mesh sizes, attributed to incorporation of a crosslinker that can provide lower crosslink densities at equivalent NCO:OH ratio, for sustained release of larger molecules such as, for example, peptides, oligonucleotides, proteins, or monoclonal antibodies, which would otherwise remain largely entrapped within the tightly crosslinked polymer matrix.

In some embodiments, the microstructures of polyester urethanes described herein are selected for use in forming smaller physical mesh sizes, attributed to incorporation of a crosslinker that can provide higher crosslink densities at equivalent NCO:OH ratio, for sustained release of small molecules, which can be challenging to deliver in a sustained manner. For example, very hydrophilic and highly water soluble small molecules can benefit from the described more densely crosslinked polyester urethane network. Further, water infiltration and permeation can be limited using the described more densely crosslinked polyester urethane network, increasing the likely of surface erosion as the degradation mechanism.

Any and all aspects of embodiments described herein may be readily combined to form alternative additional embodiments.

### EXAMPLES

The invention is further described in the context of the following examples which are presented by way of illustration, not of limitation.

The following shorthand is used for referencing polyesters and polyester urethane compositions in the following Examples. For a polyester of a formula such as P[1.1:1]G:[25:75]Suc:Seb[D], the G indicates glycerol, the Suc indicates succinic acid, the Seb indicates sebacic acid, the [1.1:1] indicates the stoichiometric ratio of glycerol:polyacid (1.1:1 in this case), the [25:75]Suc:Seb indicates two polyacids in the polyester (a polyacid of 25 mol % succinic acid and 75 mol % sebacic acid in this case), and the [D] indicates that the polyester was dialyzed after formation. For a polyester of a formula such as P[0.75:0.25:1]G:PEG300:Seb, the PEG300 represents polyethylene glycol having an average molecular weight of about 300 g/mol as a co-polyol with glycerol, in this case at a mole ratio of glycerol:PEG300:sebacic acid of 0.75:0.25:1. For a polyester of a formula such as [50:50 P[1:1]G:Ad(2 k)+P[1:1]G:Ad(8 k)]−HDI+0.1TA, the Ad indicates adipic acid, the [50:50 P[1:1]G:Ad(2 k)+P[1:1]G:Ad(8 k)] indicates a 50:50 mol:mol blend of the two indicated polyesters crosslinked by a polyisocyanate, the G:Ad indicates a single polyol and a single polyacid in the polyesters, the (2 k) and (8 k) indicate the approximate M_{w} of the first and second polyester resins prior to crosslinking (2,000 Da and 8,000 Da in this case), the −HDI indicates a polyisocyanate crosslinker (HDI in this case), and the +0.1TA indicates the presence of an added free acid (in this case 0.1 wt % tartaric acid based on the weight of the polyester urethane). For a polyester urethane of a formula such as P[1:1]G:Ad(8 k)−[2:1]HDI:LDI, the −[2:1]HDI:LDI indicates the polyester was crosslinked with a mixture of more than one polyisocyanate (in this case a 2:1 mol:mol ratio of HDI to LDI. The isocyanate-to-hydroxyl stoichiometric ratio in the Examples was approximately 1:1.

### Example 1

Twenty-seven different polyester resins of a copolymer of glycerol and one or more polyacids were synthesized by a water-mediated polycondensation process in a 30 L reactor. The weight-average molecular weight (M_{w}) and the polydispersity index (PDI) were determined for each resin by GPC RI. The results are shown in Table 1.

TABLE 1

| Resin | Name | M_{w} (g/mol) | PDI |
|---|---|---|---|
| 1 | P[1:1]G:Seb(15k) | 15103 | 8.5 |
| 2 | P[1:1]G:Seb(25k)[D] | 25279 | 4.2 |
| 3 | P[1:1]G:[95:5]Suc:Seb | 2420 | 2.3 |
| 4 | P[1:1]G:[50:50]Suc:Seb | 8442 | 4.8 |
| 5 | P[1:1]G:[25:75]Suc:Seb | 5589 | 4.0 |
| 6 | P[1:1]G:Ad(2k) | 2417 | 2.3 |
| 7 | P[1:1]G:Ad(8k) | 8927 | 5.1 |
| 8 | P[1:1]G:Ad(13k) | 13572 | 7.1 |
| 9 | P[1.1:1]G:Seb(8k) | 8189 | 4.0 |
| 10 | P[1.1:1]G:Seb(5k) | 5583 | 4.8 |
| 11 | P[1.1:1]G:Ad | 5403 | 3.8 |
| 12 | P[1.1:1]G:Ad[D] | 10041 | 2.09 |
| 13 | P[1:1]G:[95:5]Ad:Seb | 6956 | 4.1 |
| 14 | P[1:1]G:[50:50]Ad:Seb | 8187 | 4.4 |
| 15 | P[1:1]G:[25:75]Ad:Seb | 4068 | 3.2 |
| 16 | P[1.1:1]G:[50:50]Ad:Seb | 5783 | 3.7 |
| 17 | P[1.1:1]G:[50:50]Ad:Seb[D] | 5836 | 2.8 |
| 18 | P[1.1:1]G:Seb(6k) | 6241 | 4.2 |
| 19 | P[1:1]G:Seb(6k) | 6458 | 5.0 |
| 20 | P[1:1]G:Seb(12k) | 12155 | 6.96 |
| 21 | P[1:1]G:Seb(1k) | 1170 | 8.3 |
| 22 | P[1.1:1]G:[50:50]Suc:Seb | 5949 | 3.7 |
| 23 | P[1.1:1]G:[50:50]Suc:Seb[D] | 5963 | 3.5 |
| 24 | P[0.75:0.25:1]G:PEG300:Seb | 8111 | 4.3 |
| 25 | P[0.75:0.25:1]G:PEG300:Ad | 10421 | 5.8 |
| 26 | P[0.5:0.5:1]G:PEG300:Seb | 4805 | 2.4 |
| 27 | P[0.5:0.5:1]G:PEG300:Ad | 5423 | 3.2 |

The M_{w} values reported in Table 1 are for un-dried PGS resin. Resin 1 is a polyester resin for a conventional PGSU. Polyester resins 2, 12, 17, and 23 were dialyzed (indicated by [D]) after polycondensation. The measured M_{w} values ranged from 1170 to 25279 g/mol and PDI values ranged from 2.3 to 8.5.

### Example 2

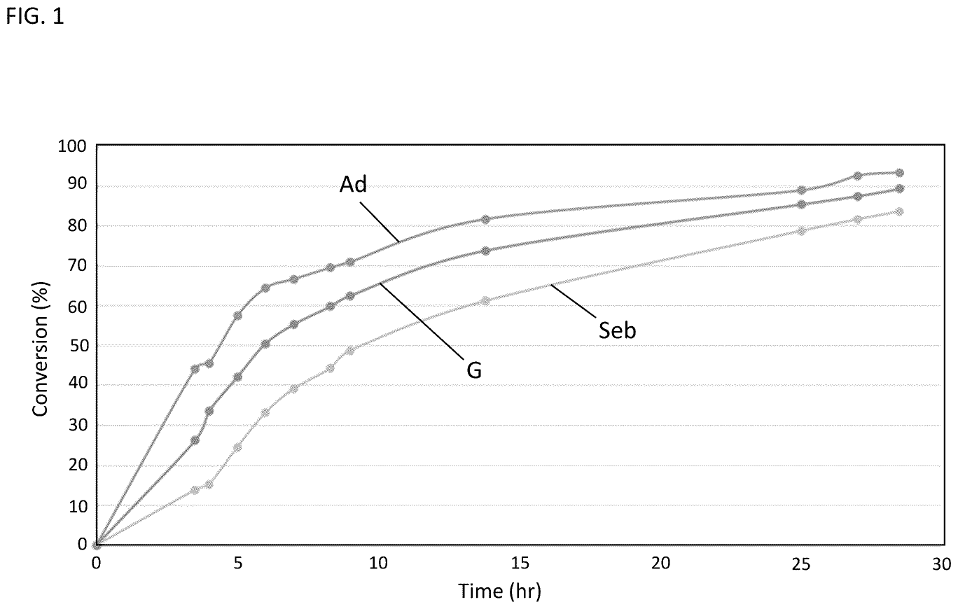

The conversion of glycerol (G), sebacic acid (Seb), and adipic acid (Ad) was monitored during polycondensation to form P[1:1]G:[50:50]Ad:Seb. FIG. 1 shows that the adipic acid reacted with the glycerol significantly faster than the sebacic acid.

### Example 3

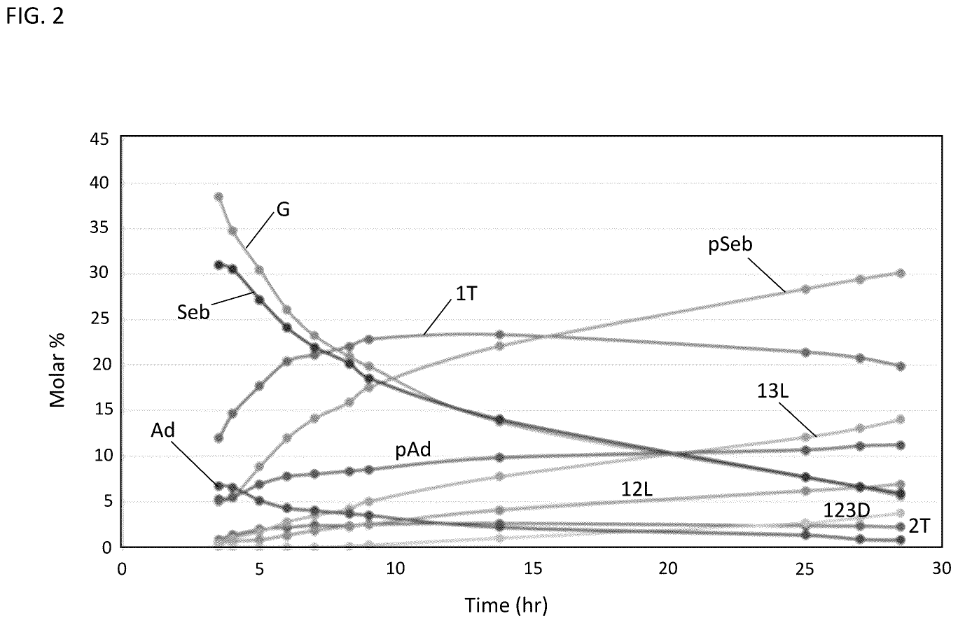

Structural parameters and the relative molar fraction of each glyceridic unit in certain polyester resins were determined during polycondensation. The polyester resin was sampled over time throughout synthesis to capture the progression of polycondensation and microstructure. ¹H-NMR data collected for each set of samples revealed the content of each possible monomer within the glycerol-based polymer over time with sebacic acid, succinic acid, and/or adipic acid as the diacid. FIG. 2 shows the evolution of free glycerol (G), free sebacic acid (Seb), free adipic acid (Ad), reacted sebacic acid (pSeb), reacted adipic acid (pAd), 1-acylglyceride (1T), 2-acylglyceride (2T), 1,3-diacylglyceride (13L), 1,2-diacylglyceride (12L), and 1,2,3-diacylglyceride (123D) over time during polycondensation to form PGSAd.

The structural parameters include the percent of carboxylic acid conversion (p_{COOH}), the glycerol degree of substitution (DS), the percentage of glycerol hydroxyl conversion (p_{OH}), and the number average degree of polymerization (DPₙ). The average molar fraction of each glyceridic species (1-acylglyceride (1T), 2-acylglyceride (2T), 1,3-diacylglyceride (1,3L), 1,2-diacylglyceride (1,2L), and 1,2,3-diacylglyceride (1,2,3D)), including nonreacted glycerol (Xᵢ), and the relative molar fraction of each glyceridic unit in the polymer chain (Yᵢ) were also determined. The results at the last measured time point for each polyester are shown in Table 2.

TABLE 2

| Resin | | 4 | 6 | 8 | 9 | 11 | 14 |
|---|---|---|---|---|---|---|---|
| Time (hr) | | 36 | 24 | 36 | 36 | 36 | 36 |
| p_{COOH1} | | 89.7^{a} | 88.1 | 92.4 | 92.0 | 97.7 | 94.4^{c} |
| p_{COOH2} | | 73.5^{b} | N/A | N/A | N/A | N/A | N/A |
| p_{OH} | | 61.8 | 59.1 | 63.9 | 60.8 | 60.3 | 61.8 |
| DPₙ | | 5.6 | 6.1 | 12.7 | 7.9 | 9.2 | 10.9 |
| Xᵢ | 1T | 24.8 | 28.2 | 22.0 | 25.2 | 25.8 | 25.8 |
| | 2T | 2.7 | 3.5 | 2.9 | 2.8 | 3.4 | 3.2 |
| | 1,2L | 39.3 | 37.5 | 37.3 | 39.3 | 36.7 | 34.5 |
| | 1,3L | 15.9 | 15.6 | 17.7 | 14.7 | 16.1 | 17.6 |
| | 1,2,3D | 13.4 | 10.1 | 16.7 | 11.5 | 11.2 | 15.0 |
| | glycerol | 3.9 | 5.1 | 3.3 | 6.5 | 6.9 | 4.0 |
| Yᵢ | 1T | 25.8 | 29.7 | 22.8 | 26.9 | 27.7 | 26.8 |
| | 2T | 2.8 | 3.6 | 3.0 | 3.0 | 3.6 | 3.3 |
| | 1,2L | 40.9 | 39.5 | 38.6 | 42.0 | 39.5 | 35.9 |
| | 1,3L | 16.6 | 16.4 | 18.3 | 15.7 | 17.2 | 18.3 |
| | 1,2,3D | 13.9 | 10.7 | 17.3 | 12.3 | 12.0 | 15.6 |

^{a}sebacic acid;
^{b}succinate;
^{c}average of the diacids

p_{COOH}, p_{OH}, and DPₙ increased with reaction time. 1T and 2T decreased with reaction time after the initial measurement at 12 hours. 1,3L, 1,2L, and 1,2,3D increased with reaction time.

### Example 4

Table 3 shows the relative molar ratios of microstructures of certain polyester resins from Example 1 as determined by ¹³C NMR spectroscopy. The appropriate instrument settings were used to provide sufficient peak resolution allowing quantitative determination of all peaks related to the acylglyceride moiety. The degree of branching reported in Table 3 was calculated by integrating the peaks in the glyceride region, representing 1T, 2T, 1,2L, 1,3L, and 1,2,3D, and then obtaining the area under each respective peak and determining the percentage of each of the peaks compared to total glyceride peaks in polymer. Samples were prepared on deuterated acetone at a concentration of 500 mg/mL. The degree of branching was calculated from the equation degree of branching=2D/(2D+L), where D is the amount of 1,2,3D and L is the combined amount of 1,2L and 1,3L.

TABLE 3

| | | | | | | Degree of |
|---|---|---|---|---|---|---|
| Resin | 1T | 2T | 1,2L | 1,3L | 1,2,3D | Branching |
| 1 | 27.27 | 3.2 | 15.84 | 37.23 | 16.45 | 0.38 |
| 2 | 23.39 | 2.4 | 16.2 | 40.16 | 17.85 | 0.39 |
| 6 | 34.3 | 4.5 | 14.9 | 29.1 | 9.7 | 0.31 |
| 7 | 29.6 | 3.03 | 16.7 | 32.8 | 14.1 | 0.35 |
| 8 | 25.5 | 3.1 | 18.6 | 33.7 | 15.8 | 0.37 |
| 9 | 34.96 | 3.25 | 17.89 | 34.14 | 9.76 | 0.27 |
| 10 | 39.4 | 4.43 | 14.62 | 31.75 | 9.8 | 0.3 |
| 11 | 33.9 | 3.2 | 14.5 | 30.7 | 9.7 | 0.3 |
| 12 | 21.3 | 2.6 | 20.8 | 37.8 | 17.4 | 0.37 |
| 14 | 31.1 | 4.9 | 18 | 32.8 | 13.1 | 0.34 |
| 18 | 39.42 | 2.92 | 11.68 | 33.58 | 12.41 | 0.35 |
| 19 | 31.93 | 4.2 | 17.65 | 33.61 | 12.61 | 0.33 |

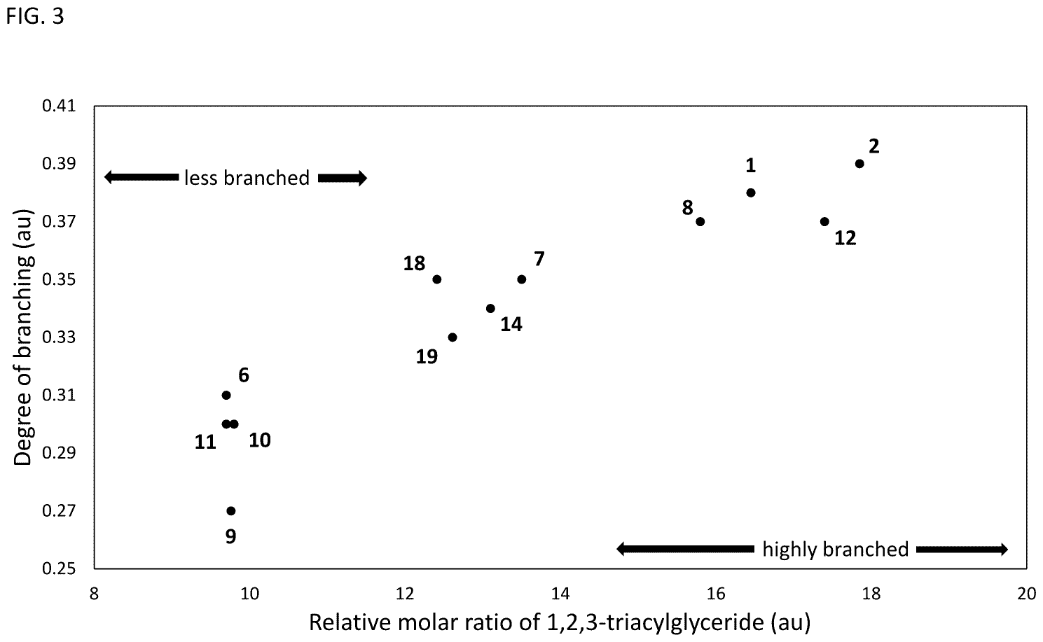

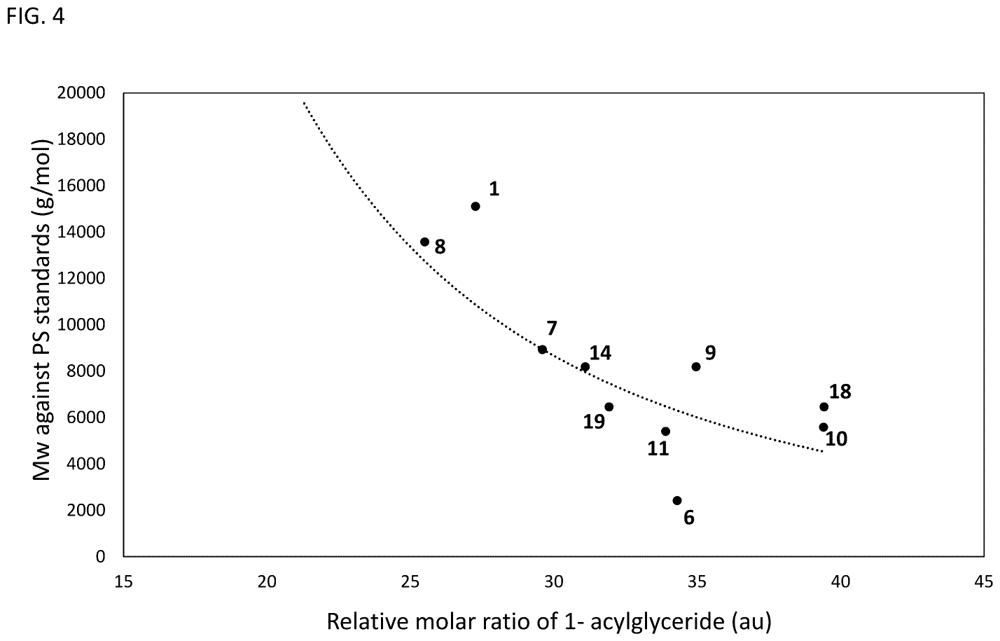

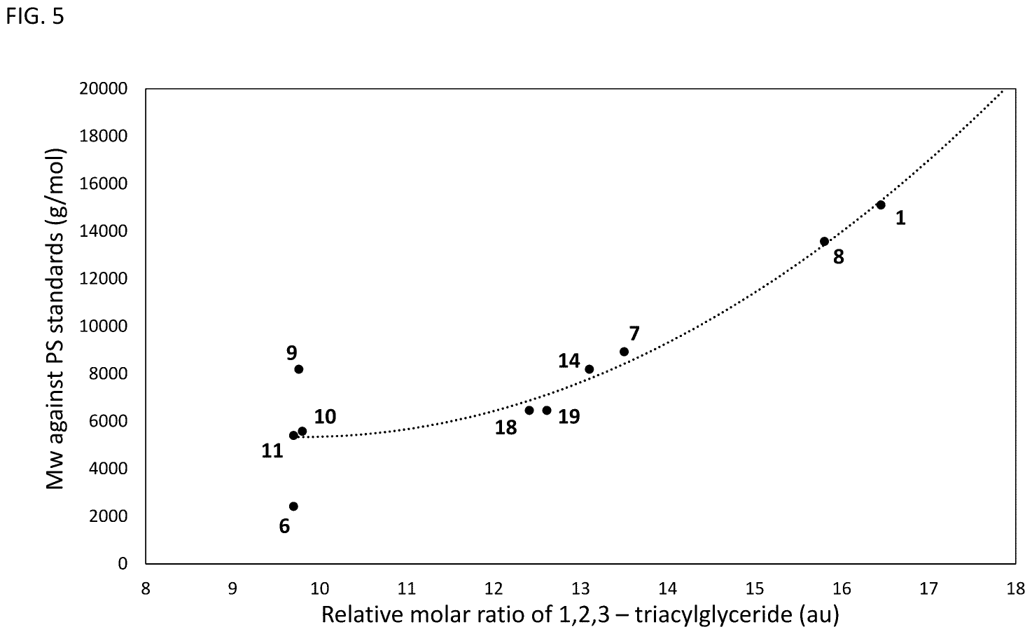

FIG. 3 shows a plot of the degree of branching of polyester resins versus the relative molar ratio of 1,2,3-triacylglyceride. FIG. 1 shows the segregation of different degrees of branched resins being mainly governed by the amount of 1,2,3-triacylglyceride units and molecular weight. FIG. 4 shows a plot of the weight-average molecular weight of polyester resins versus the relative molar ratio of 1-acylglyceride, with the ratio decreasing with increasing molecular weight. FIG. 5 shows a plot of the weight-average molecular weight of polyester resins versus the relative molar ratio of 1,2,3-triacylglyceride, with the ratio decreasing with increasing molecular weight.

### Example 5

Size exclusion chromatography refractive index multiple angle light scattering (SEC-RI-MALS) was used to measure the molecular weight of PG:Seb polyester resins at varying timepoints during polycondensation synthesis and molecular weights were obtained compared to PS standards using the RI detector.

### Example 6

Radar plots were prepared to show nine different physiochemical parameters for various selected polyester urethane formulations. The nine parameters were the M_{w} of the polyester in Da, the polydispersity index of the polyester, the mol % of 1,2,3-acylglyceride of the polyester, the average diacid solubility of the diacid in mg/mL, the average diacid hydrocarbon length (average number of CH₂ groups between carboxylic acids), the hydroxyl value of the polyester in mg KOH/g, the acid value of the polyester in mg KOH/g, the rotational flow viscosity of the polyester in Pa·s, and the log(P) value of the isocyanate.

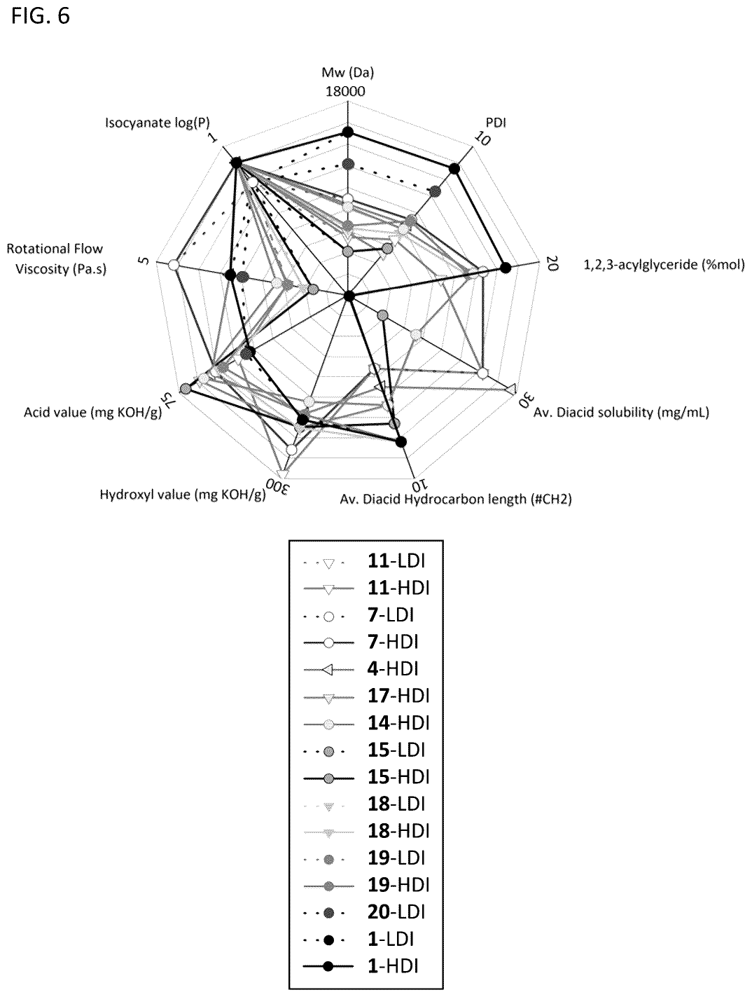

FIG. 6 shows a radar plot of polyester urethanes having polyester components formed from polyesters composed of various molecular weights. Polyester components were composed of a single polyol, specifically glycerol, a single or multiple polyacids, specifically sebacic acid, adipic acid, or copolymers of adipic acid:sebacic acid or succinic acid:sebacic acid. Polyester components were synthesized with either 1:1 or 1.1:1 polyol:polyacid molar ratio. Urethane components were composed of either HDI or LDI. The radar plot shows the physiochemical parameters on each radial axis, with values ranging from 0 at the center to the maximum value indicated for each axis.

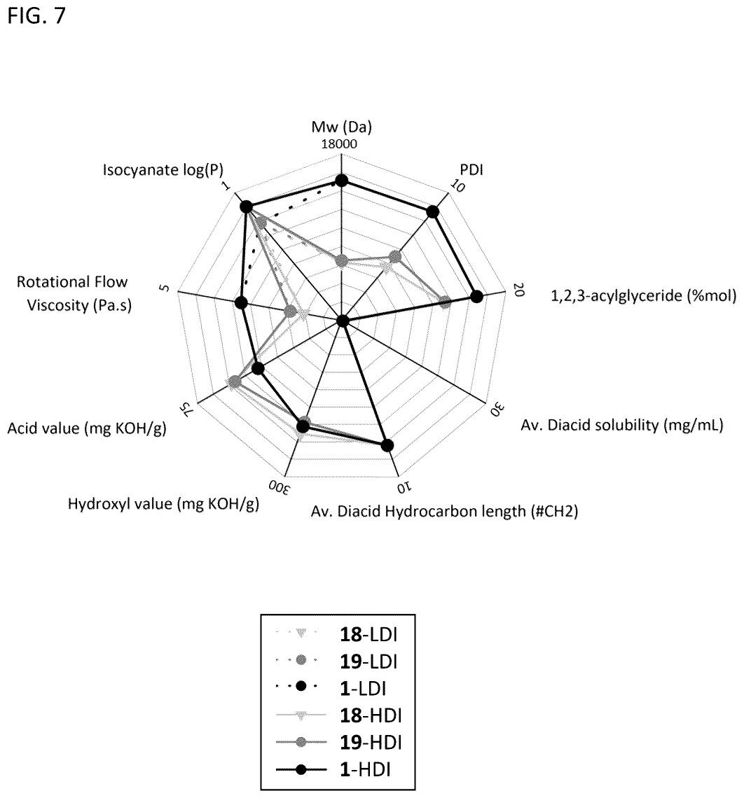

FIG. 7 shows a radar plot of PGSU formulations made using polyesters with either 1:1 (circle) or 1.1:1 (downward triangle) polyol:polyacid molar ratio and with either HDI (solid line) or LDI (dotted line).

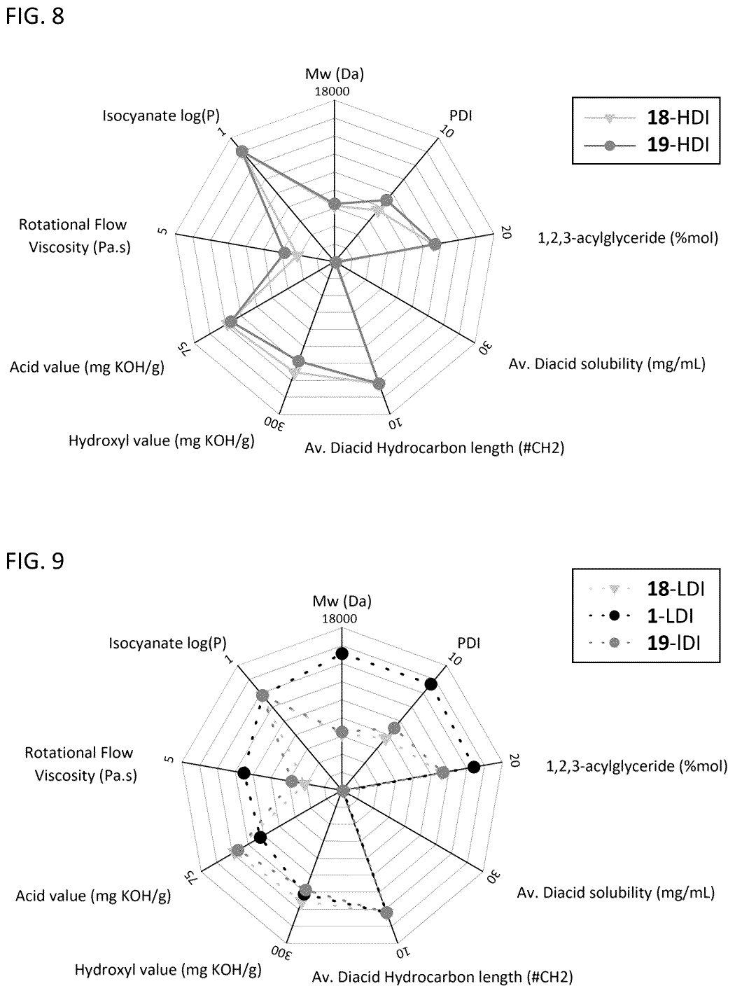

FIG. 8 shows a radar plot of PGSU formulations made using polyesters with either 1:1 (circle) or 1.1:1 (downward triangle) polyol:polyacid molar ratio and with HDI.

FIG. 9 shows a radar plot of PGSU formulations made using polyesters with either 1:1 (circle) or 1.1:1 (downward triangle) polyol:polyacid molar ratio and with LDI.

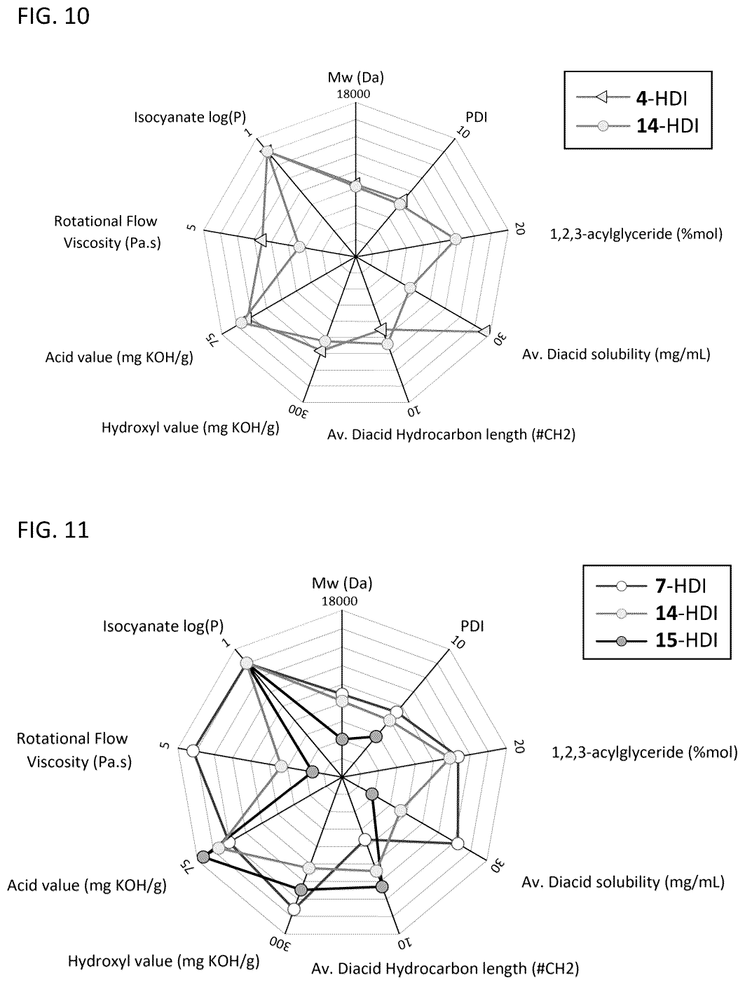

FIG. 10 shows a radar plot of PGSU formulations made using polyesters with either adipic acid (circle) or succinic acid (sideways triangle) with sebacic acid and with HDI. Polyacid copolymers of adipic acid:sebacic acid or succinic acid:sebacic acid were 50:50 molar ratio. Polyesters were synthesized with 1:1 polyol:polyacid molar ratio.

FIG. 11 shows a radar plot of PGSU formulations made using polyesters with either adipic acid (white circle) or polyacid copolymers of adipic acid with sebacic acid at adipic acid:sebacic acid molar ratios of 50:50 (light gray circle) or 25:75 (dark gray circle) and with HDI. Polyesters were synthesized with 1:1 polyol:polyacid molar ratio.

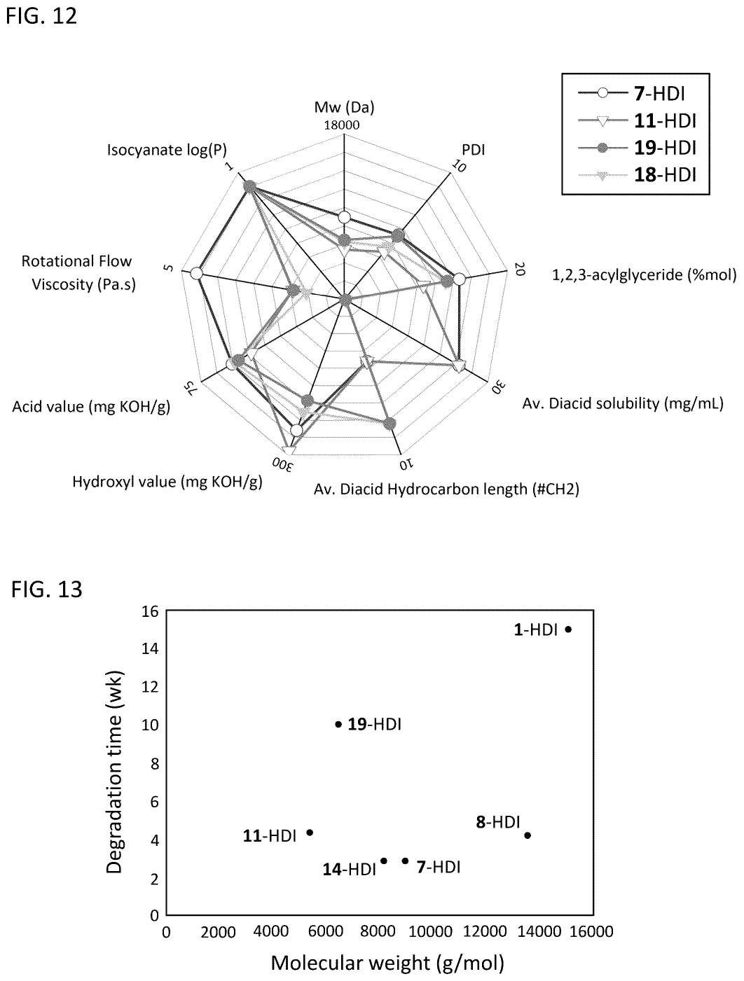

FIG. 12 shows a radar plot of PGSU formulations made using polyesters with either adipic acid (white markers) or sebacic acid (gray markers), either 1:1 (circle) or 1.1:1 (downward triangle) polyol:polyacid molar ratio, and with HDI.

### Example 7

Degradation profiles were obtained for implants of unloaded polyester urethanes crosslinked by HDI, the polyester resins varying in their polyol:polyacid molar ratio, molecular weight, diacid component, and/or present of a free acid, for comparison to conventional PGSU (1-HDI), in 0.1 M PBS at the accelerated degradation condition of 70° C. The implants were cylindrical with a diameter of 5 mm and a height of 5 mm. At each timepoint, three samples of each polyester urethane were washed in de-ionized water and dried at 40° C. under vacuum for 48 hours before being weighed. The results, in mass %, are shown in Table 4, showing that a wide range of degradation rates are possible with HDI as the crosslinker without varying the ratio of crosslinker to polyester.

TABLE 4

| PEU | 0 wks | 2 wks | 3 wks | 4 wks |
|---|---|---|---|---|
| 1-HDI | 0 | 6.2 | N/D | 24 |
| 3-HDI | 0 | N/D | N/D | 100 |
| 4-HDI | 0 | 34.4 | 80.2 | 100 |
| 8-HDI | 0 | N/D | N/D | 93 |
| 11-HDI | 0 | N/D | N/D | 80 |
| 14-HDI | 0 | 35.2 | 100 | — |
| 16-HDI | 0 | N/D | N/D | 55 |
| 16-HDI + 0.1% TA | 0 | N/D | N/D | 75 |
| 18-HDI + 0.1% TA | 0 | N/D | N/D | 65 |
| 22-HDI | 0 | N/D | N/D | 63 |

The polyester urethane containing adipic acid, 14-HDI, degraded fully in just 3.26 weeks, which is five times faster than conventional PGSU. Unexpectedly, the polyester urethane containing succinic acid, 4-HDI, degraded at a slower rate than the adipic-acid containing equivalent despite being a shorter diacid, but this 3.42 weeks was still four times faster than the time for conventional PGSU to degrade completely.

### Example 8

Degradation profiles were obtained for implants of unloaded polyester urethanes crosslinked by HDI, the polyester resins varying in their polyol:polyacid molar ratio, molecular weight, and/or diacid component, under the same experimental procedure as for Example 7. Results for conventional PGSU, 1-HDI are included for reference. The results at four weeks, in mass %, are shown in Table 5, showing that a wide range of degradation rates are possible with LDI as the crosslinker without varying the ratio of crosslinker to polyester.

TABLE 5

| PEU | 4 wks |
|---|---|
| 1-HDI | 24 |
| 1-LDI | 34 |
| 4-LDI | 100 |
| 9-LDI | 35 |
| 11-LDI | 100 |
| 14-LDI | 100 |
| 18-LDI | 96 |

The polyester urethane containing only adipic acid, 11-LDI, degraded fully in just 3.26 weeks, which is five times faster than conventional PGSU. The polyester urethane containing both sebacic and adipic acid, 14-LDI, degraded fully in just 2.86 weeks. The polyester urethane containing both sebacic and succinic acid, 4-LDI, degraded fully in just 2.57 weeks.

### Example 9

Degradation profiles were obtained for implants of unloaded polyester urethanes containing varying proportions of sebacic acid and adipic acid under the same experimental procedure as for Example 7. Results for conventional PGSU are included for reference. The results, in mass %, are shown in Table 6.

TABLE 6

| PEU | 0 wks | 2 wks | 3 wks | 4 wks |
|---|---|---|---|---|
| 1-HDI | 0 | 6.2 | N/D | 24.2 |
| 13-HDI | 0 | 27.1 | N/D | 100^{a} |
| 14-HDI | 0 | 35.2 | 100 | |
| 15-HDI | 0 | 14.8 | N/D | 54.7 |

^{a}13-HDI was 100% degraded at 3.43 weeks

Table 6 shows that the degradation profile of the polymer can be tuned by adjusting the adipic acid content between 0 to 50%. Unexpectedly, constructs with 95% adipic acid degraded slightly slower than equivalent constructs with 50% adipic acid, suggesting an ideal range of tunability.

### Example 10

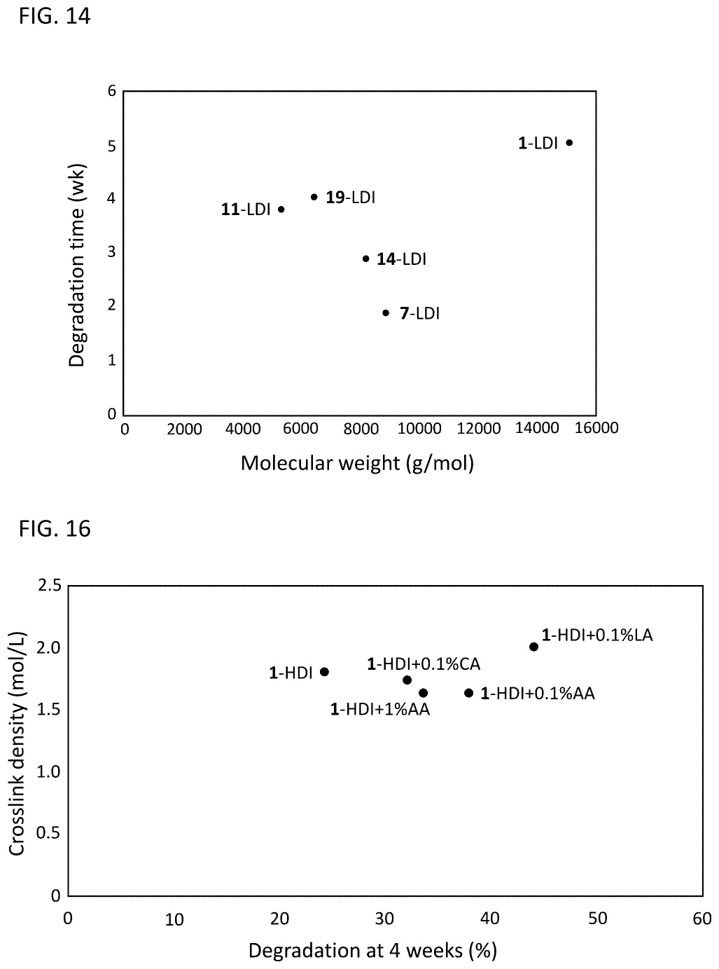

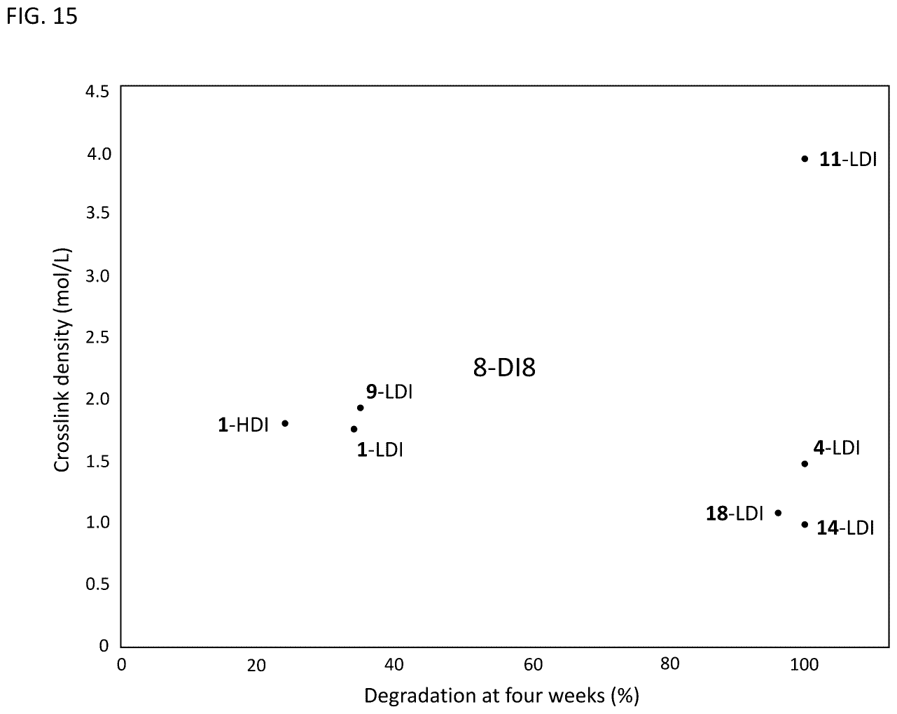

Degradation times at 70° C. were obtained for implants of unloaded polyester urethanes containing varying proportions of sebacic acid and adipic acid under the same experimental procedure as for Example 7. Results for conventional PGSU are included for reference. The results with HDI as the crosslinker are plotted as degradation time in weeks versus molecular weight of the polyester resin in FIG. 13. The results with LDI as the crosslinker are plotted as degradation time in weeks versus molecular weight of the polyester resin in FIG. 14. FIG. 15 shows the degradation at four weeks versus the crosslink density for a variety of LDI-crosslinked polyester urethanes compared to 1-HDI.

### Example 11

Degradation profiles were obtained for implants of polyester urethanes doped with a free acid, either tartaric acid (TA), citric acid (CA), lactic acid (LA), or acetic acid (AA) under the same experimental procedure as for Example 7. Results for conventional PGSU are included for reference. The pre-polymer was mixed with the free acids prior to crosslinking. The amounts of free acid are by weight percent. The results, in mass %, are shown in Table 7. FIG. 16 shows the degradation at four weeks versus the crosslink density of these polyester urethanes.

TABLE 7

| PEU | 0 wks | 2 wks | 4 wks |
|---|---|---|---|
| 1-HDI | 0 | 6.2 | 24.2 |
| 1-HDI + 0.1% TA | 0 | 13.2 | 56.9 |
| 1-HDI + 0.1% CA | 0 | 9.0 | 32.0 |
| 1-HDI + 0.1% LA | 0 | 10.2 | 44.0 |
| 1-HDI + 0.1% AA | 0 | 10.6 | 37.9 |
| 1-HDI + 1% AA | 0 | 9.0 | 33.6 |

Table 7 shows that tartaric acid addition resulted in significantly higher degradation rates compared to other acids at an equivalent wt %. Surprisingly no negative effect to the crosslink density was observed at these amounts, although the addition of the acid enhanced hydrolysis, as expected. A higher wt % of tartaric acid did not result in a higher degradation rate and in some cases led to excessive bubble formation within the implants. Free acids, such as tartaric acid, act as a catalyst for the hydrolysis of ester bonds and lead to a significantly more hydrolyzable polymer network.

### Example 12

Degradation profiles were obtained for implants of polyester urethanes including adipic and sebacic acid and either doped with TA or undoped under the same experimental procedure as for Example 7. Results for conventional PGSU are included for reference. The polyester resin was mixed with the free acids prior to crosslinking. The amount of free acid is by weight percent. The results, in mass %, are shown in Table 8.

TABLE 8

| PEU | 0 wks | 2 wks | 4 wks |
|---|---|---|---|
| 1-HDI | 0 | 6.2 | 24.2 |
| 16-HDI | 0 | 14.2 | 54.9 |
| 16-HDI + 0.1% TA | 0 | 9.0 | 75.3 |

Table 8 shows that the addition of free 0.1% tartaric acid provided a similar significant increase in the degradation rate of polyester urethanes including adipic and sebacic acid as it did for a conventional PGSU.

### Example 13

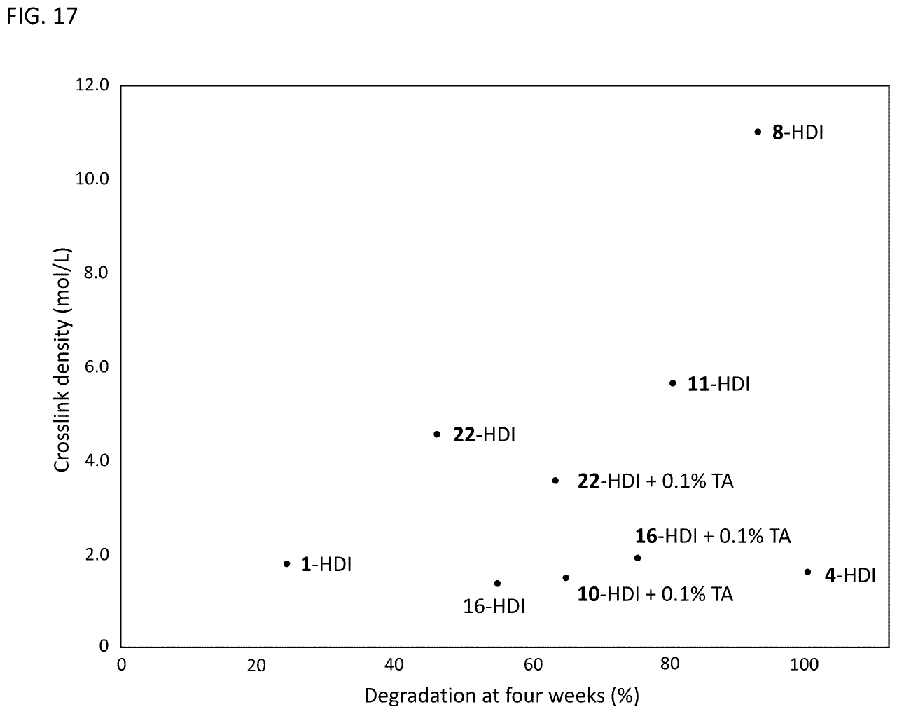

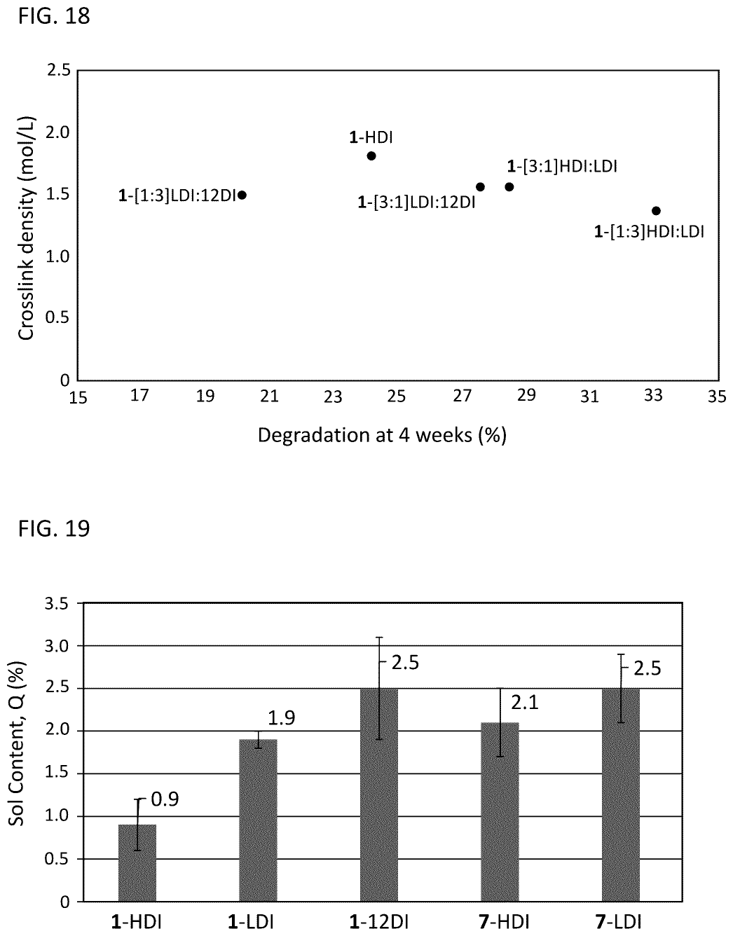

Degradation of unloaded polyester urethanes crosslinked by HDI was measured at four weeks. FIG. 17 shows a plot of crosslink density against degradation percentage after four weeks. Even at a very high crosslink density (8-HDI), PGAdU degraded significantly faster than PGSU (1-HDI), which was unexpected. FIG. 18 shows that a range of degradation rates can be achieved without significantly changing the crosslink density by using a mixture of crosslinkers.

### Example 14

Sol content (Q, %) was determined for certain polyester urethanes from 3-mm by 40-mm rods in THF for 24 hours at room temperature. FIG. 19 shows that the sol content increases for polyester 1 when the crosslinker is changed from HDI to LDI and 12DI. It is suspected that the lowered crosslink densities of the LDI and 12DI formulations cause this effect. A similar effect is seen with formulations made using polyester 7. A low sol content suggests that there are lower levels of extractables in the formulation.

### Example 15

7-HDI broke into pieces during an accelerated 70° C. degradation study, breaking gradually over time into more and more, smaller and smaller pieces. In contrast, 21-HDI, where pre-vac PGS (Resin 21) was low molecular weight and collected prior to the vacuum step during polycondensation, remained in a single piece throughout mass loss and diameter loss over time. The mechanism of degradation can play an important role in product performance and patient acceptability. For example, for intravitreal implants, it would be undesirable to have an implant degrade into multiple pieces, which can cause issues with vision, lymphatic drainage, tear duct drainage, inflammation, and foreign body response, among other negative physiological effects.

### Example 16

Degradation profiles were obtained for implants of polyester urethanes, formed from a blend of two polyester resins, under the same experimental procedure as for Example 7. Results for conventional PGSU are included for reference. The polyester resins were blended together in equal amounts by weight overnight prior to crosslinking. The results, in mass %, are shown in Table 9.

TABLE 9

| PEU | 0 wks | 2 wks | 4 wks |
|---|---|---|---|
| 1-HDI | 0 | 6.2 | 24.2 |
| [1 + 3] − HDI | 0 | 46.8 | 63.8 |
| [13 + 18] − HDI | 0 | 17.0 | 54.3 |
| [13 + 18] − LDI | 0 | 25.3 | 94.2 |

Ad:Seb resins blended with PGS more uniformly and resulted in a urethane elastomer that degraded homogeneously and faster than conventional PGSU, as shown by Table 9. Suc:Seb resins showed some signs of de-mixing and phase separation from PGS resins upon blending and degraded non-homogeneously after urethane crosslinking, as shown by their mass loss profile and degradation topography.

### Example 17

Degradation profiles were obtained for implants of polyester urethanes, formed using different HDI:LDI ratios, under the same experimental procedure as for Example 7. Results for conventional PGSU are included for reference. The results, in mass %, are shown in Table 10.

TABLE 10

| Polyester urethane | 0 wks | 2 wks | 4 wks |
|---|---|---|---|
| 1-HDI | 0 | 6.2 | 24.2 |
| 1-[3:1]HDI:LDI | 0 | 10.8 | 28.4 |
| 1-[1:3]HDI:LDI | 0 | 13.9 | 33.0 |
| 1-[3:1]LDI:12DI | 0 | N/D | 27.6 |
| 1-[1:3]LDI:12DI | 0 | N/D | 20.2 |

Table 10 shows that a more solubilizing crosslinker, such as LDI, can be used in combination with HDI to tune the degradation kinetics of polyester urethanes. This effect is closely related to the difference in molecular weight of the degradants formed using the different crosslinkers.

### Example 18

Degradation profiles were obtained for implants of polyester urethanes, formed using a diacid other than sebacic acid and/or a crosslinker other than HDI, under the same experimental procedure as for Example 7. Results for conventional PGSU are included for reference. The results, in mass %, are shown in Table 11.

TABLE 11

| Polyester urethane | 0 wks | 2 wks | 4 wks |
|---|---|---|---|
| 1-HDI | 0 | 6.2 | 24.2 |
| 7-HDI | 0 | 23.1 | 80.3 |
| 7-LDI | 0 | 23.1 | 100^{a} |

^{a}7-LDI was 100% degraded at 3.29 weeks

Table 11 shows that a more solubilizing crosslinker, such as LDI, can be used to tune the degradation kinetics of polyester urethanes with different diacids, such as adipic acid, and with glycerol:diacid ratios other than the conventional 1:1.

### Example 19

Degradation profiles were obtained for implants of polyester urethanes, formed using a diacid mix of sebacic acid and adipic or succinic acid and a crosslinker of HDI or LDI, under the same experimental procedure as for Example 7. Results for conventional PGSU are included for reference. The results, in mass %, are shown in Table 12.

TABLE 12

| Polyester urethane | 0 wks | 2 wks | 3 wks | 4 wks |
|---|---|---|---|---|
| 1-HDI | 0 | 6.2 | N/D | 24.2 |
| 4-HDI | 0 | 34.4 | 80.2 | 100 |
| 14-HDI | 0 | 35.2 | 100 | |
| 4-LDI | 0 | 54.0 | 100^{a} | |
| 14-LDI | 0 | 39.8 | 100^{b} | |

^{a}4-LDI was 100% degraded at 2.57 weeks;
^{b}14-LDI was fully degraded at 2.86 weeks

Table 12 shows that the degradation advantages of LDI over HDI are greater in some polymer formulations than others depending on the composition of the soft segment. The Ad:Seb polyester urethane had a very similar degradation profile with the two different crosslinker, whereas the Suc:Seb polyester urethane had significantly faster degradation profile with LDI than with HDI.

### Example 20

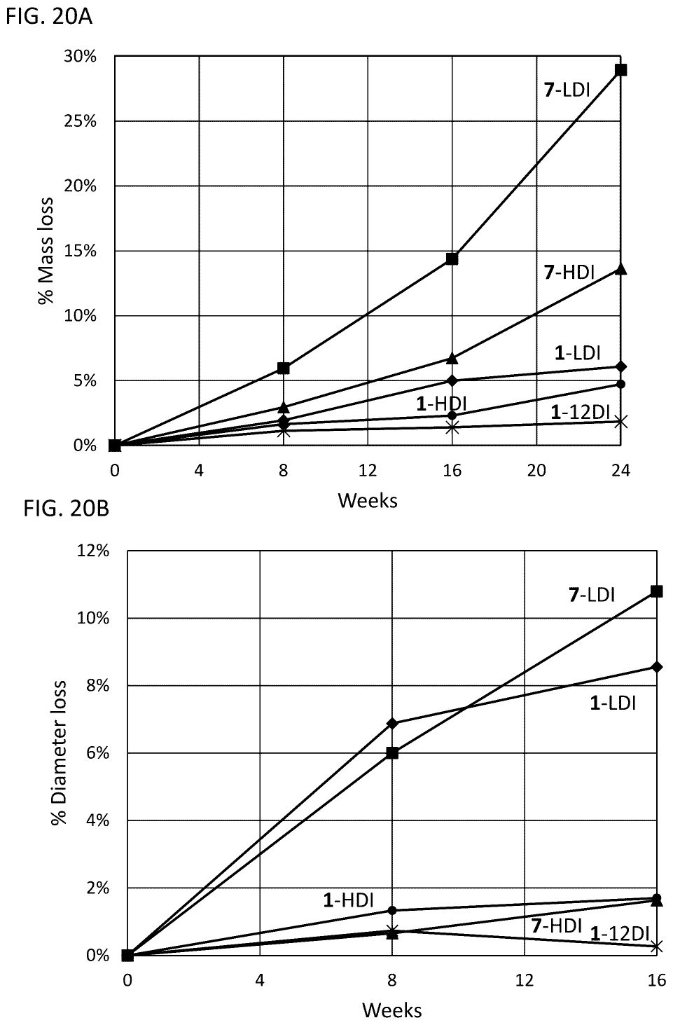

Degradation profiles were obtained for implants of polyester urethanes, formed with sebacic acid or adipic acid as the diacid and a crosslinker of HDI or LDI, under the same experimental procedure as for Example 7 except molded at 3-mm diameter and 10-mm length and degraded at a temperature of 37° C. Additionally, the diameter was measured at eight and 16 weeks and the diameter loss was calculated. Results for conventional PGSU are included for reference. The mass loss results are shown in FIG. 20A. The diameter loss results are shown in FIG. 20B.

7-HDI and 7-LDI exhibited significant swelling during degradation at 37° C., where an originally 3-mm implant swelled to 3.4 mm and 3.9 mm by 16 weeks, respectively. When dried, 7-LDI showed a 340-μm diameter loss, while 7-HDI only showed a 50-μm diameter loss. In contrast, 1-LDI exhibited less swelling than 7-LDI, yet showed similar diameter loss at 8 weeks, approximately 200 μm, and at 16 weeks, approximately 260 μm. At 16 weeks, 7-LDI demonstrated the most mass loss, followed by 7-HDI, then 1-LDI. 1-LDI, however, was the only one where mass loss and diameter loss were better synchronized for surface erosion as the degradation mechanism.

The mass loss data and dimensional loss data at 37° C. confirm the findings at 70° C., showing that the incorporation of adipic acid increases the degradation rate of polyester urethanes compared to conventional PGSU. The degradation rate can also be tune by changing the hydrophilicity of the crosslinking from the most hydrophobic, 12DI, to the most hydrophilic, LDI.

### Example 21

P[1.1:1]G:[50:50]Suc:Seb (Resin 22) was also dialyzed to P[1.1:1]G:[50:50]Suc:Seb[D] (Resin 23) for similar purposes. The hydroxyl value decreased from 262 to 215 mg KOH/g. Interestingly, the acid value remained the same at 55 mg KOH/g. Yield following dialysis was approximately 70 to 85 wt %. With the intentionally reduced hydroxyl value following hydrolysis, not as much isocyanate content needed to be added to the formulation to maintain equivalent isocyanate-to-hydroxyl stoichiometric equivalence, in contrast to the P[1.1:1]G:Ad-HDI examples. Even still, the dialyzed 17-HDI formulations degraded more slowly than 14-HDI formulations despite having only a slightly higher crosslink density by Flory-Rehner swelling of 1.725 mol/L vs. 1.377 mol/L. Also, the dialyzed 17-HDI degraded faster than non-dialyzed 16-HDI despite having a higher resin molecular weight, although it did have a lower crosslink density of 1.725 mol/L versus 2.516 mol/L. Moreover, dialyzed 17-HDI sustained release better than 14-HDI. These results together speak to the importance of 1:1 versus 1.1:1 polyol:polyacid stoichiometric ratio, as well as the impact of hydroxyl groups, carboxylate groups, and monomers and oligomers in the starting polymer resin.

### Example 22

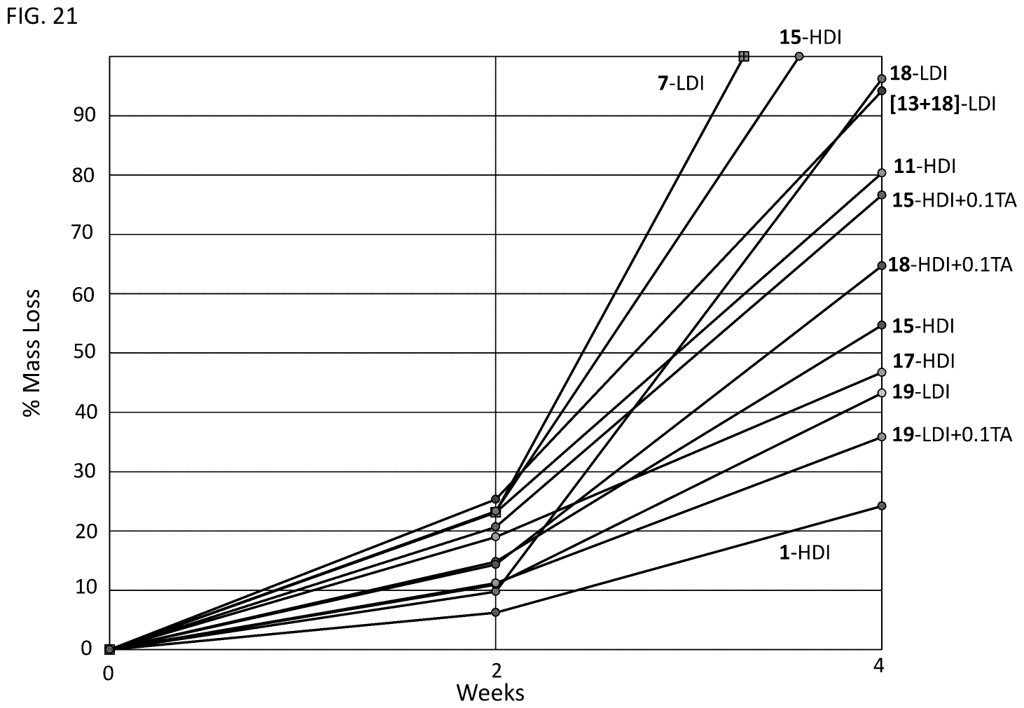

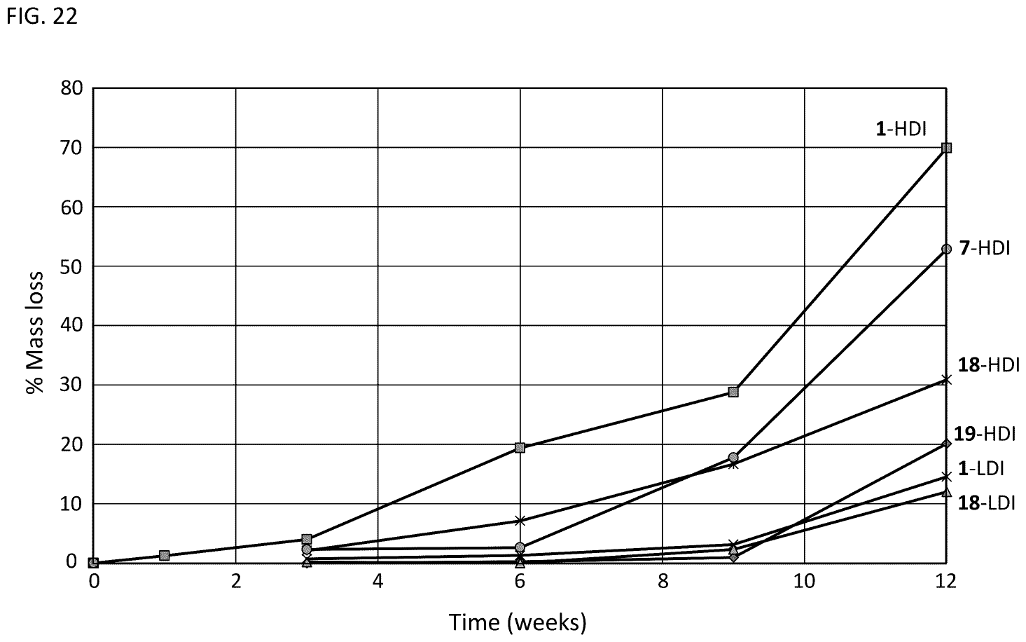

FIG. 21 shows degradation profiles for tested implants of polyester urethanes of interest. FIG. 22 shows degradation profiles for tested implants of additional polyester urethanes of interest. Results for conventional PGSU are included for reference. This group includes urethanes from various polyester resins including a range of molecular weights, sebacic acid, adipic acid, or a combination of the two as the diacid, and a polyester resin blend, with HDI or LDI as the crosslinker, and several doped with a free acid.

This range of polymers highlights the impact of using different diacids, diacid to glycerol ratio, crosslinker, and molecular weight of pre-polymer in tuning the degradation profile of polyester urethanes over a large range of timeframes.

### Example 23

Polyester resins of Example 1 were crosslinked with either HDI or LDI. The resulting polyester urethane implants were subjected to degradation, with comparison to conventional PGSU (1-HDI), in 0.1 M PBS at the accelerated degradation condition of 70° C. The implants were cylindrical with a diameter of 5 mm and a height of 5 mm. SEC spectra of the degradation products were used to determine their weight-average and number average molecular weight. The M_{w}, Mₙ, and PDI values, as determined by GPC RI, are given in Table 13.

TABLE 13

| PEU | Crosslinker | M_{w} (g/mol) | Mₙ (g/mol) | PDI |
|---|---|---|---|---|
| 1 | HDI | 545 | 387 | 1.406 |
| 4 | HDI | 1690 | 539 | 3.133 |
| 13 | HDI | 1470 | 161 | 9.148 |
| 14 | HDI | 421 | 100 | 4.201 |
| 18 | HDI | 1691 | 726 | 2.33 |
| 1 | LDI | 13004 | 1455 | 8.94 |
| 4 | LDI | 36428 | 1449 | 25.145 |
| 11 | LDI | 1548 | 222 | 6.962 |
| 14 | LDI | 28606 | 1941 | 14.741 |
| 18 | LDI | 10893 | 329 | 33.1 |

The polyester urethanes with LDI as the crosslinker had significantly higher M_{w} values than those with HDI as the crosslinker, with the exception of LDI when adipic acid was the only diacid. In that case the M_{w} value was similar to the polyester urethanes with HDI as the crosslinker. The increasing M_{w}, Mₙ, and PDI values are indicative of increased solubility of larger degradants, and a significant amount and range of small-to-medium sized soluble degradants are still present when large soluble degradants are present.

### Example 24

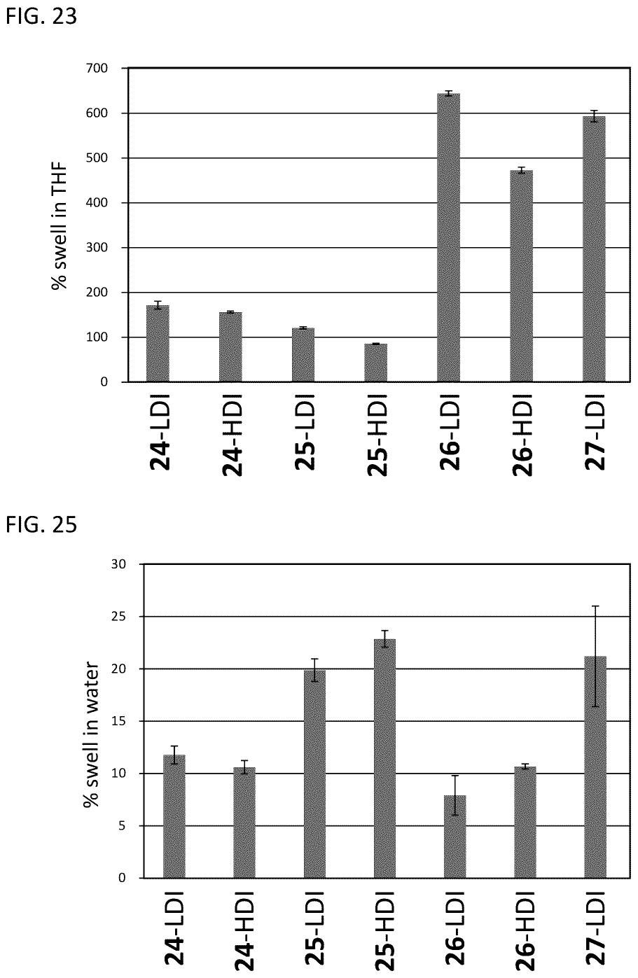

The percentage of swelling of certain unloaded polyester urethanes was determined in both THF and water to evaluate their hydrophilicity. These polymers included polyester resins that included both glycerol and polyethylene glycol of 300 g/mol (PEG300) as polyols, which allowed modulation of the hydrophilicity of the polymer. The results are shown in FIG. 23 for THF after 24 hours at room temperature and FIG. 25 for water after seven days at room temperature. Polyester urethanes including PEG300 showed a higher uptake of water over seven days compared to the base glycerol polymer formulations. Additionally, going from P[0.75:0.25:1]G:PEG300:Seb to P[0.75:0.25:1]G:PEG300:Ad, there was a significant increase in the water uptake, which shows that the type of diacid used in the polyester resin also has a strong effect on the uptake of water.

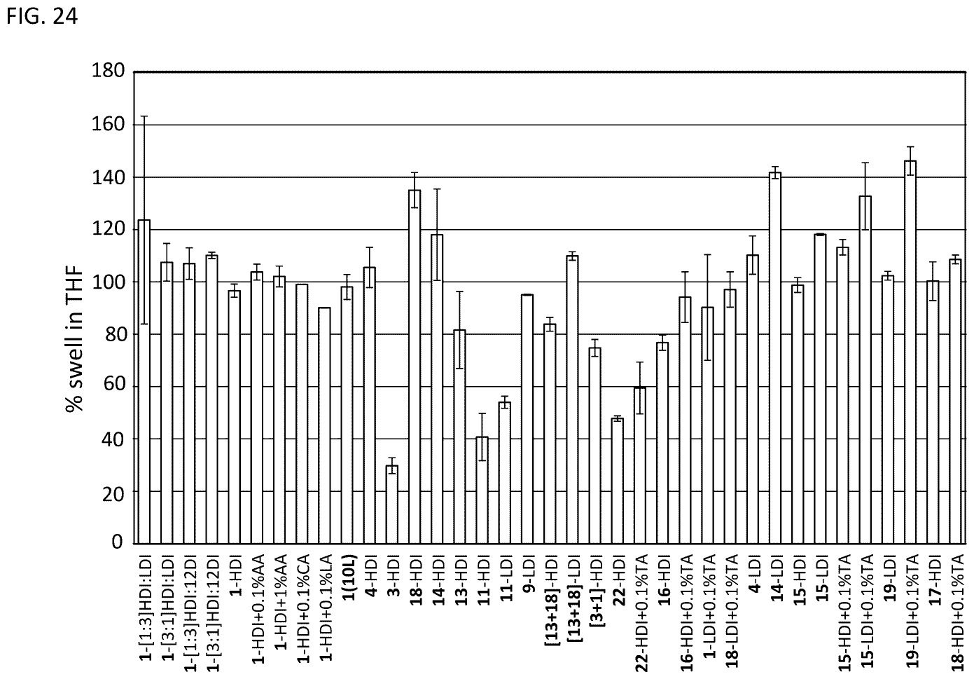

Swelling results in THF for additional polyester urethanes are shown after 24 hours at room temperature in Table 14 and FIG. 24.

TABLE 14

| Polyester urethane | Swelling in THF (%) |
|---|---|
| 1-HDI | 87.6 |
| 1-LDI | 119.4 |
| 1-12DI | 97.9 |
| 7-HDI | 73.3 |
| 7-LDI | 98.9 |

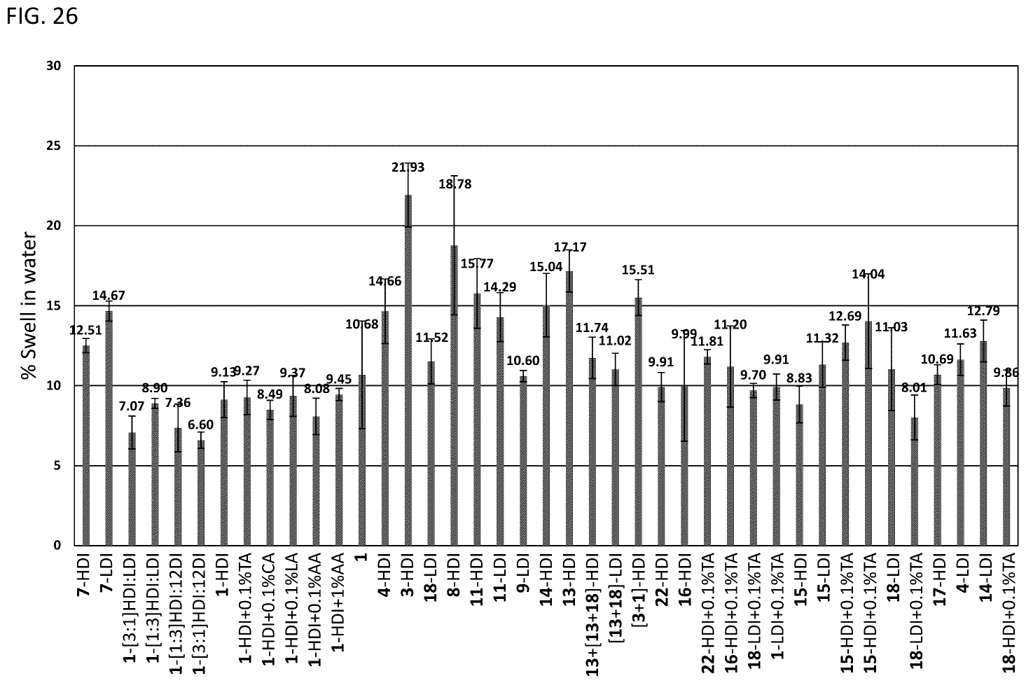

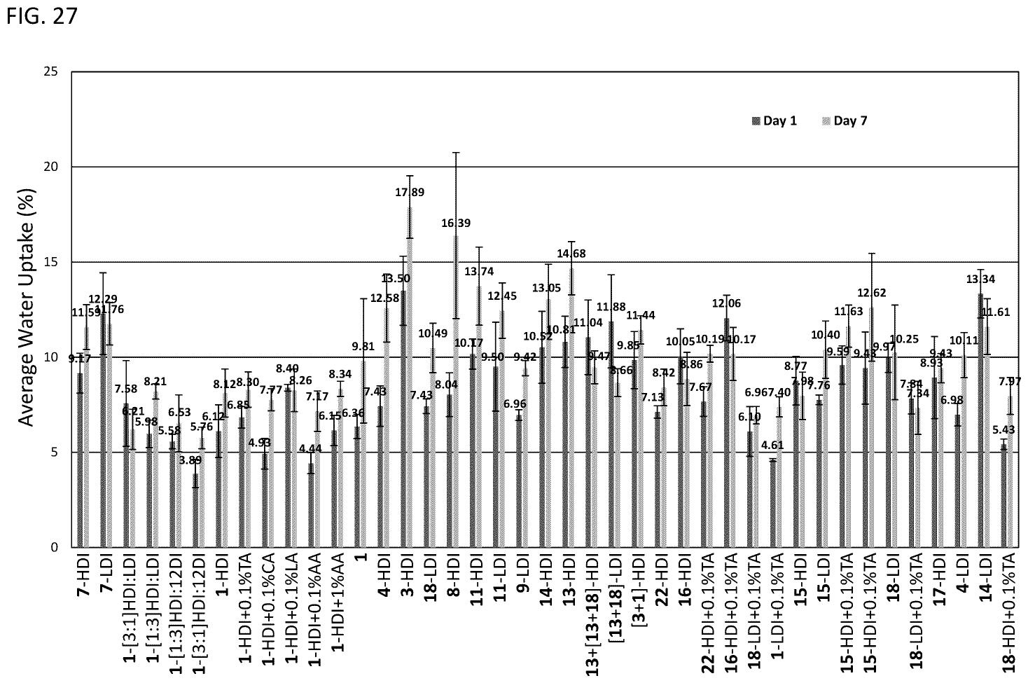

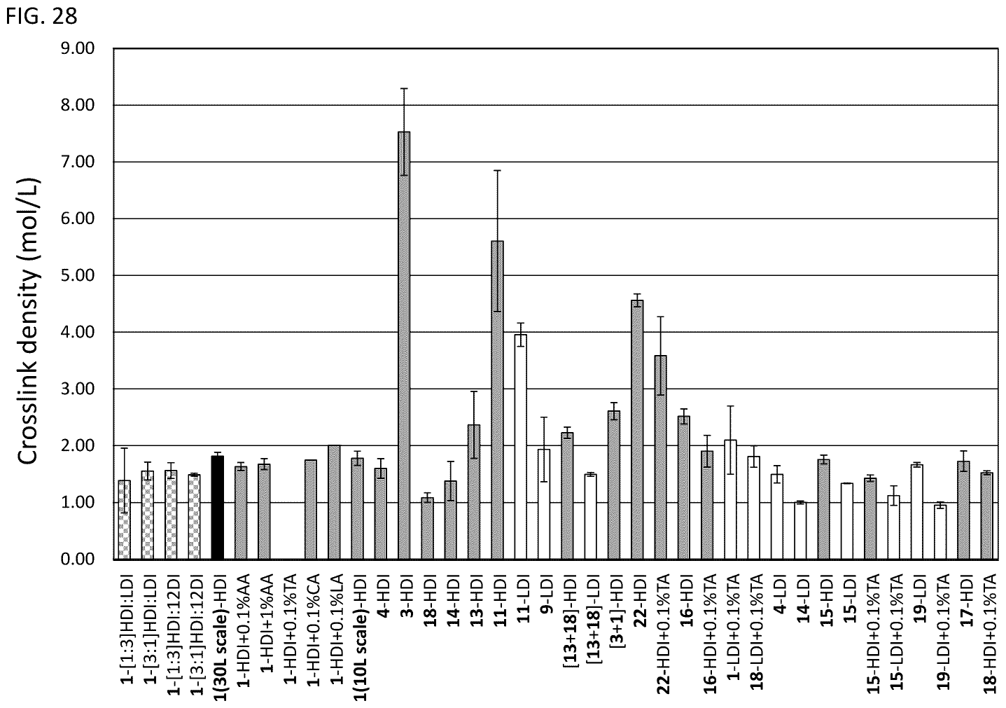

Swelling results in water after seven days at room temperature for additional polyester urethanes are shown in FIG. 26. Water uptake after one day and after seven days at room temperature for certain polyester urethanes is shown in FIG. 27.

The crosslink density of polyester urethanes was determined by Flory-Rehner solvent swelling in THF. The bar for conventional PGSU (1(30L scale)-HDI from a Resin 1 made on a 30-L scale) is black. The bars for HDI-crosslinked polymers are gray, including a 1(10L scale)-HDI from a Resin 1 made on a 10-L scale with a similar crosslink density to the 1(30L scale)-HDI. The bars for LDI-crosslinked polymers are white. The bars for combinations of crosslinkers are checkered. These included combinations of HDI, LDI, or 12DI. Some polyester urethanes included acid additives at either 0.1% or 1%. These additives included acetic acid (AA), tartaric acid (TA), citric acid (CA), and lactic acid (LA). The results are shown in FIG. 28.

An equivalent isocyanate-to-hydroxyl ratio of 1:1 to 1.1:1 was applied to 1-LDI formulations as well as 1-HDI formulations. Unexpectedly, the 1-LDI had a lower crosslink density of the bulk network, as tested by Flory-Rechner solvent swelling, of about 1 mol/L compared to 1-HDI which was about 2 mol/L. Implant samples tested were the same batch as those described in Example 20.

P[1.1:1]G:Ad(5 k)-HDI (polyester resin 11) synthesized with a 1.1:1 glycerol:adipic acid stoichiometric ratio and approximately 5,000 Da M_{w} was formulated with either LDI or HDI at equivalent isocyanate-to-hydroxyl ratio of 1:1 to 1.1:1. The 11-LDI had a higher crosslink density of the bulk network, as tested by Flory-Rehner solvent swelling, compared to 1-LDI, 1-HDI, or 18-HDI. The 11-HDI had the highest crosslink density of all, even higher crosslink density than when crosslinked with LDI, as tested by Flory-Rechner solvent swelling. The crosslink density as empirically measured by Flory-Rechner solvent swelling does not predict degradation time in these formulations. Polyester resin 11 formulated with either LDI or HDI demonstrated more than 2.5-times or 3.5-times higher crosslink density, respectively, per solvent swelling, yet degraded significantly faster than 1-HDI.

The high hydroxyl value of polyester resin 11 was matched with an increase in isocyanate during formulation to maintain the stoichiometric equivalent ratio. While this led to a very dense crosslinked network that was not swellable, it unexpectedly degraded more quickly due to the soft segment polyacid composition. This may be the effect of polyglycerol adipate regions being more impermeable to THF, which is the solvent in which the swell experiments are done (see also Godinho et al.). This was consistent with what was observed with polymers made of succinic acid as well. However, the rate of degradation in water-based media was unexpected and suggests that swell and crosslinking in THF, a semipolar solvent, does not necessarily correlate to the polymer behavior in water. It is very interesting that the effect expected with thermoset resin can be mimicked even when the resin is crosslinked with isocyanates.

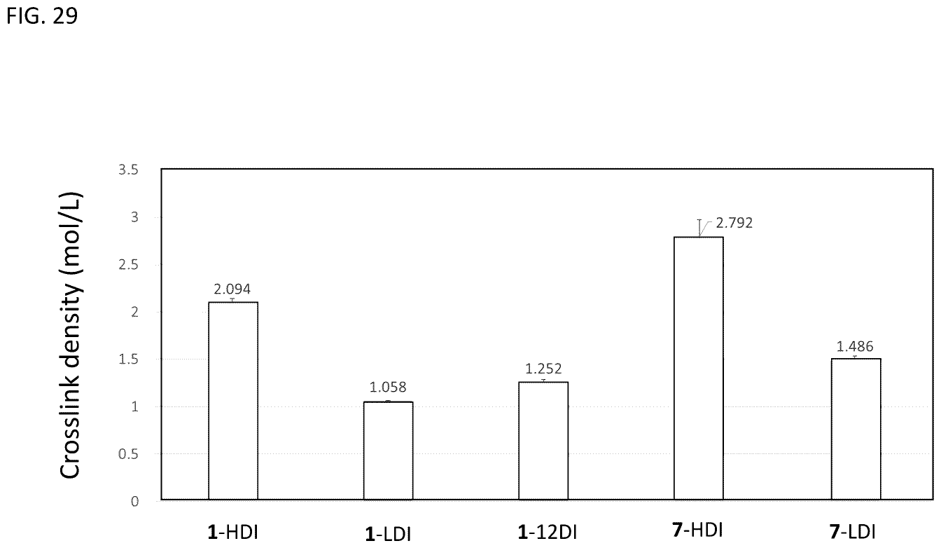

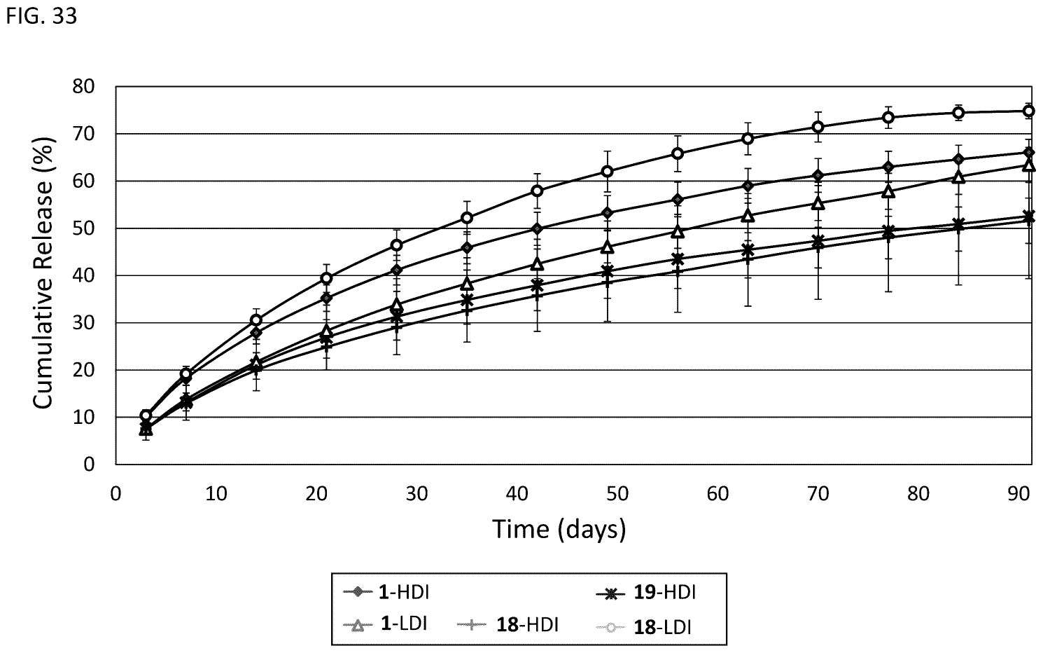

Moreover, release of dexamethasone loaded in the polyester urethane was unexpectedly faster than other formulations with lower crosslink density, again due to the soft segment polyacid composition. FIG. 29 shows that 1-LDI demonstrated lower crosslinking than 1-HDI, so a faster degradation would be expected, but its more sustained release of dexamethasone was unexpected (see Example 32 and FIG. 33). These results speak to the importance of the microstructure, which is more impactful than solely looking at isocyanate-to-hydroxyl ratio or crosslink density to predict performance.

### Example 25

10% 1,4-butanediol was added as a chain extender to 30 wt % hydrophilic 2′-deoxyadenosine loaded 1-HDI. 100% of the 2′-deoxyadenosine was released within 14 days in simulated gastric fluid (SGF) at a pH of 1.2 at 37° C. as compared to 28 days for 100% release of 2′-deoxyadenosine under the same conditions from solvent-free 1-HDI without chain extenders. 1-HDI with 10% 1,4-butanediol lost 38% polymer mass and all mechanical integrity and turned into a resinous material in 2 weeks at a pH of 1.2 and 37° C. as opposed to solvent-free 1-HDI which took greater than 12 weeks.

### Example 26

In one example, a 15 mass % loss was observed from a 30-40% hydrophilic-API loaded 1-HDI with 10% 1,4-butanediol in 21 days as opposed to negligible mass loss in solvent-free 1-HDI.

### Example 27

In one example, 30 wt % 2′-deoxyadenosine loaded 1-HDI with 5 wt %, 7.5 wt %, and 10 wt % 1,5-pentanediol, showed 6%, 10%, and 10% polymer mass loss by day 7 in SGF at pH 1.2 and 37° C. 30 wt % 2′-deoxyadenosine loaded 1-HDI with 5 wt %, 7.5 wt %, and 10 wt % 1,5-pentanediol, showed 9%, 12%, and 13% polymer mass loss by day 21 in SGF at pH 1.2 and 37° C. The polymer mass loss plateaued after day 21. It is possible that since the chain extender is added to the polyester resin followed by addition of the isocyanate, there is reaction between the isocyanate and chain extender. This decreases the crosslinking of the polyester resin by the isocyanate and the final PGSU may have phase-separated regions with chain extender-isocyanate repeating units. When exposed to aqueous release media like SGF, this isocyanate-chain extender unit may be lost rapidly which coincides with the rapid mass loss for the first few weeks. After this rapid initial loss, the PGSU left behind has a longer degradation time.

### Example 28

Microdevices of polyester urethanes loaded with 60 wt % dexamethasone were analyzed for mass loss by extraction at one month, three months, 6 months, 9 months, and 12 months in a 37° C., 50 RPM release/degradation study in PBS/saline. The microdevices were cylindrical in shape with a 450-μm diameter and a 10-mm length. For extraction, samples were extracted in dimethyl sulfoxide for 4 hours/400 RPM at room temperature. Extraction media was diluted 1:10 in 50:50 acetonitrile:Type 1 water and processed by HPLC through an Agilent Zorbax C18 reverse-phase column for detection of dexamethasone, along with a DMSO blank and a 0.5-mg/ml dexamethasone control. Dexamethasone concentrations were calculated based on a dexamethasone standard curve (R2-0.9999) and used to verify initial dexamethasone loading and quantify total drug release. The post-release masses of washed and lyophilized microrods were used to calculate the mass % loss attributed to polymer degradation only by subtracting the amount of dexamethasone release. The mass loss was attributed only to polyester urethane component. The faster hydrolysis of the shorter diacids is observed even in the presence of dexamethasone. The results are shown in Table 15.

TABLE 15

| Polyester | 1 month | 3 months | 6 months | 9 months | 12 months |
|---|---|---|---|---|---|
| urethane | (mass %) | (mass %) | (mass %) | (mass %) | (mass %) |
| 7-HDI | 1.24 | 3.99 | 19.37 | 28.76 | 69.90 |
| 18-HDI | N/D | 0.17 | 0.00 | 2.31 | 12.00 |
| 1-LDI | N/D | 2.08 | 7.13 | 16.68 | 30.89 |
| 19-HDI | N/D | 0.70 | 1.30 | 3.12 | 14.53 |
| 18-LDI | N/D | 2.28 | 2.61 | 17.88 | 52.84 |
| 1-HDI | N/D | 0.00 | 0.23 | 0.95 | 20.13 |

Table 15 shows that polyester urethanes with adipic acid as the polyacid degraded faster than polyester urethanes with sebacic acid, highlighting the effect of frequency of esterification within the polyol despite equivalent isocyanate-to-hydroxyl stoichiometric ratios. Polyester urethanes with LDI as the isocyanate degraded faster than polyester urethanes with HDI. Furthermore, sebacic acid based polyols with a low molecular weight, when formulated into polyester urethanes, such as 18-HDI and 19-HDI, exhibited faster initial mass loss between 3-9 months due to solubilization of shorter chains. However, long-term degradation at 12 months revealed that formulations with higher molecular weight polyols overtook the lower molecular weight polyols in terms of mass loss. This effect is attributed to the microstructural organization of the lower molecular weight polyol chains, compared to the amorphous organization of the higher molecular weight polyol. The hydrophobic HDI crosslinker is then able to stabilize the polyol microstructure through formation of urethane linkages, resulting in the unique degradation behavior that was observed.

### Example 29

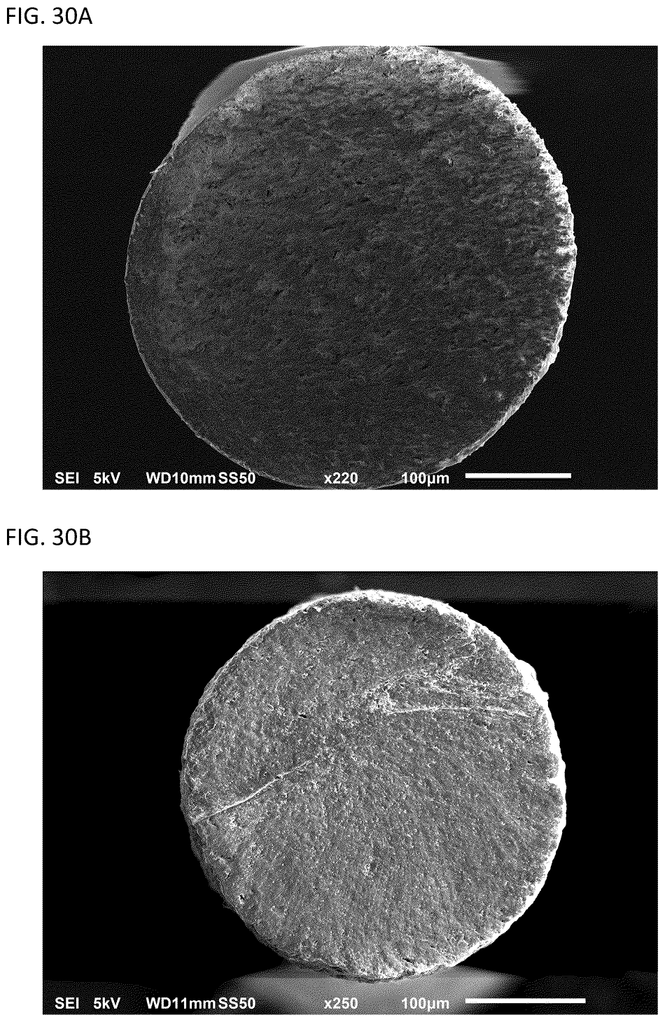

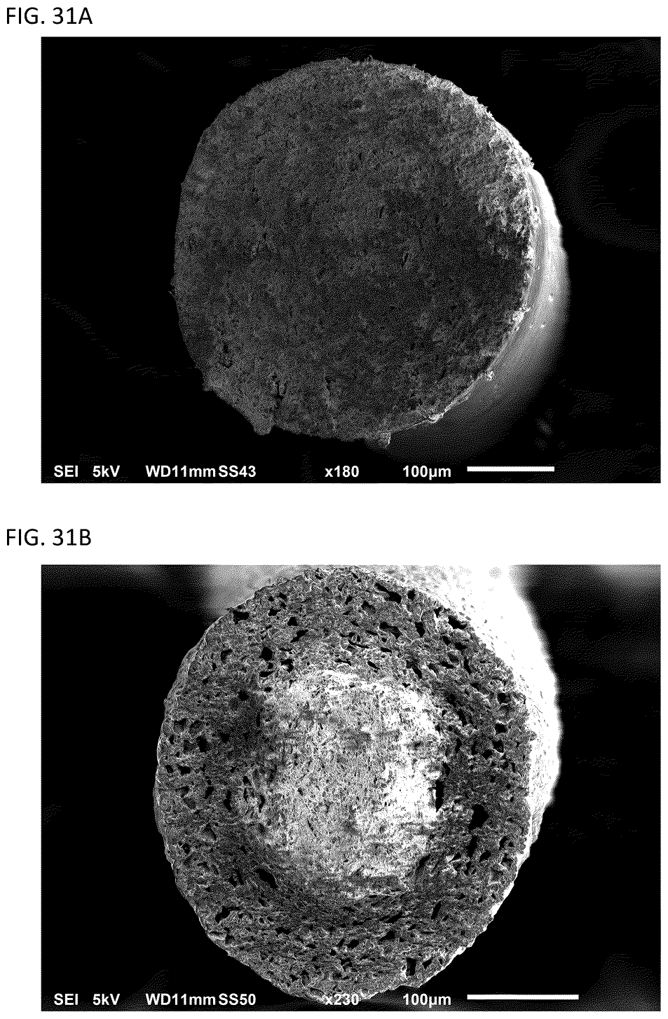

Two of the microdevices of Example 28 were imaged by SEM prior to the degradation study and after the three-month degradation study after drying by lyophilization. FIG. 30A shows 7-HDI before the degradation study. FIG. 30B shows 7-HDI after the degradation study and lyophilization. FIG. 31A shows 18-HDI before the degradation study. FIG. 31B shows 18-HDI after the degradation study and lyophilization.

Larger pores in the 18-HDI formulation after three months may be attributed to high degree of dexamethasone aggregation during the formulation/curing process. Although there are differences between the two formulations, the lyophilization may have caused pore collapse with more degradation or softer mechanical properties of the polyester urethane.

### Example 30

Dexamethasone was well-distributed in PGSU (1-HDI) at t=0, and as drug released at 37° C., no large pores were observed as the drug evacuated. In contrast, dexamethasone acetate loaded in 1-HDI left behind large pores as the drug evacuated. This may be due to dexamethasone acetate being more hydrophobic and having a partition coefficient log(P) of 2.6 compared to dexamethasone log(P) of 1.93. Dexamethasone solubility is around 75 μg/mL, whereas dexamethasone acetate has a worse aqueous solubility around 5 μg/mL, in PBS at 25° C.

### Example 31

In another example, dexamethasone was well-distributed in 1-LDI at t=0, and as drug released at 37° C., no large pores were observed as the drug evacuated. In contrast, dexamethasone loaded in low molecular weight, low branching 1-HDI left behind large pores as the drug evacuated. This may be due to dexamethasone behavior differences in the two different soft segments and/or the two different hard segments. The log(P) of HDI is 0.89, while the log(P) of LDI is 0.76. Drug release, mass loss, diameter loss, and surface roughness were mostly similar between the two formulations at the 3-month timepoint, although later timepoints may illustrate differences. Both formulations demonstrated 100-μm diameter loss from their original diameter, by measuring the samples at 3 months after drying, although 1-LDI demonstrated a larger swollen diameter at 3 months.

### Example 32

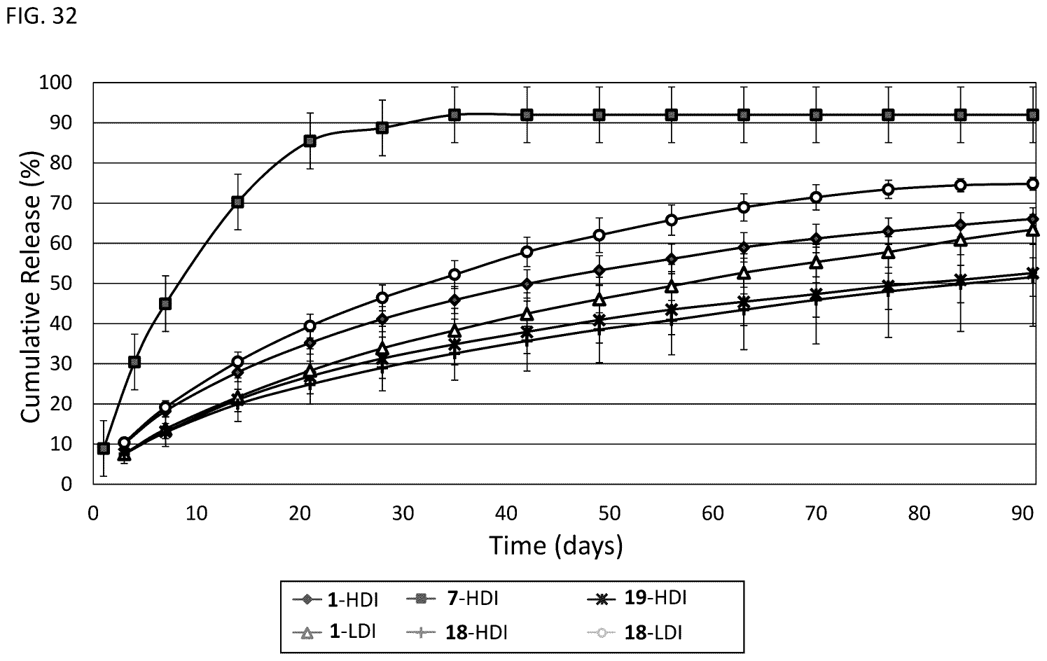

Cumulative release profiles were measured for polyester urethane microdevices loaded with 60 wt % dexamethasone similar to those of Example 28 (n=9 for each formulation). Release of dexamethasone was evaluated in 100 mL (3× sink condition) of PBS or saline at 37° C./50 RPM, sampled at 3 or 4 days and then weekly thereafter with complete media changes. Release media was analyzed for dexamethasone by HPLC on an Agilent 1260 Infinity II system with an Agilent Zorbax C18 reverse-phase column, calculating concentration with a dexamethasone standard curve (R2-0.9999). The mobile phase includes a gradient of Type 1 with 0.1% formic acid and HPLC-grade acetonitrile with 0.1% formic acid at a flow rate of 1 mL/min. Cumulative release was calculated by converting release concentration to mass released and dividing by the total initial mass of dexamethasone. FIG. 32 shows the resulting release profiles. 7-HDI provided the fastest release, significantly faster than the other five polyester urethanes with sebacic acid as the polyacid.

FIG. 33 shows only the five polyester urethanes with sebacic acid. The use of LDI as the crosslinker with high molecular weight PGS resin resulted in slower release kinetics, as seen by comparing the 1-HDI and 1-LDI curves. When HDI was replaced with LDI for a low molecular weight PGS resin, the release kinetics were instead accelerated, as seen by comparing 18-HDI or 19-HDI with 18-LDI. Surprisingly, this effect was not observed when a different form of dexamethasone having a smaller particle size and smaller agglomerations was loaded, where the HDI versions degraded/released faster than the LDI versions, showing a dependency on the morphology and arrangement of the API within the polymer.

### Example 33

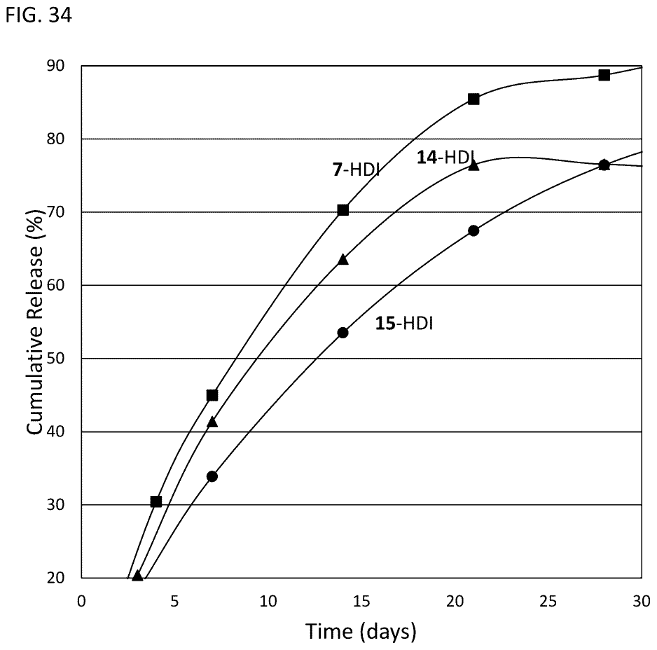

Cumulative release profiles were measured for polyester urethane microdevices loaded with 60 wt % dexamethasone similar to those of Example 32 following a similar procedure as Example 32. FIG. 34 shows the resulting release profiles. These examples highlight the ability to tune the release rate of hydrophobic molecules using adipic acid:sebacic acid ratio.

All above-mentioned references are hereby incorporated by reference herein.

While the invention has been described with reference to one or more exemplary embodiments, it will be understood by those skilled in the art that various changes may be made and equivalents may be substituted for elements thereof without departing from the scope of the invention. In addition, many modifications may be made to adapt a particular situation or material to the teachings of the invention without departing from the essential scope thereof. Therefore, it is intended that the invention not be limited to the particular embodiment disclosed as the best mode contemplated for carrying out this invention but that the invention will include all embodiments falling within the scope of the appended claims. In addition, all numerical values identified in the detailed description shall be interpreted as though the precise and approximate values are both expressly identified.

What is claimed is:

1. A composition comprising a polyester urethane, wherein the polyester urethane comprises a crosslinker based on a diisocyanate and an alternating copolymer resin of at least one polyol monomer and at least one polyacid monomer, the crosslinker crosslinking the alternating copolymer resin, the alternating copolymer resin having a degree of branching, a weight average molecular weight, a polydispersity index, and a viscosity prior to the crosslinking, the polyester urethane having a first stoichiometric ratio of the at least one polyol to the at least one polyacid, the polyester urethane having a second stoichiometric ratio of isocyanate-to-hydroxyl, the polyester urethane having a degree of phase-separated microstructure between hard segments of the crosslinker and soft segments of the alternating copolymer resin, the crosslinking stabilizing the degree of phase-separated microstructure, the degree of phase-separated microstructure being based on a selected combination of the at least one polyol monomer, the at least one polyacid monomer, the degree of branching, the weight average molecular weight, the polydispersity index, the viscosity, the diisocyanate, the first stoichiometric ratio, and the second stoichiometric ratio, the degree of phase-separated microstructure providing a predetermined property to the composition selected from the group consisting of a degradation rate in an aqueous environment, a release rate of a drug loaded in the polyester urethane in the aqueous environment, a solubility of the drug, and combinations thereof.

2. The composition of claim 1, wherein the at least one polyol monomer comprises glycerol.

3. The composition of claim 1, wherein the at least one polyacid monomer is selected from the group consisting of sebacic acid, suberic acid, adipic acid, succinic acid, and combinations thereof.

4. The composition of claim 1, wherein the diisocyanate is selected from the group consisting of hexamethylene diisocyanate and lysine diisocyanate.

5. The composition of claim 4, wherein the weight average molecular weight of the alternating copolymer resin is less than 6,500 Da.

6. The composition of claim 4, wherein the degree of branching is such that the polyester resin has a 1,2,3-triacylglyceride content of less than 15 mol %.

7. The composition of claim 4, wherein the degree of branching is such that the polyester resin has a 1,2,3-triacylglyceride content of more than or equal to 15 mol %, and the isocyanate comprises lysine diisocyanate.

8. The composition of claim 1, wherein the at least one polyol monomer comprises glycerol, the at least one polyacid comprises sebacic acid, and the isocyanate comprises lysine diisocyanate.

9. The composition of claim 8, wherein the at least one polyacid monomer further comprises adipic acid.

10. The composition of claim 1 further comprising a chain extender selected from the group consisting of 1,4-butanediol, 1,5-pentanediol, 1,6-hexanediol, isopentyldiol, 2-methy1,3-propanediol, bis(2-hydroxyethyl) terephthalate, ethylene glycol, diethylene glycol, propylene glycol, dipropylene glycol, ethylene diamine, 2-hydroxyethyl 2-hydroxypropanoate, and 2,2-bis(hydroxymethyl) propionic acid.

11. The composition of claim 10, wherein the chain extender is added in an amount of about 5 to 15 wt % with respect to a weight of the flowable polyester resin.

12. The composition of claim 1 further comprising the drug.

13. A process of forming a polyester urethane, the process comprising:
selecting at least one polyol monomer, at least one polyacid monomer, and a diisocyanate;
combining the at least one polyol monomer and an aqueous liquid in a vessel;
selecting a first stoichiometric ratio of the at least one polyol monomer to the at least one polyacid monomer and adding the at least one polyacid monomer to the vessel at the first stoichiometric ratio;
removing water from the vessel;
producing an alternating copolymer resin of the at least one polyol monomer and the at least one polyacid monomer, the alternating copolymer resin having a degree of branching, a weight average molecular weight, a polydispersity index, and a viscosity; and
selecting a second stoichiometric ratio of isocyanate-to-hydroxyl and homogeneously combining a flowable blend comprising the alternating copolymer resin with the diisocyanate at the second stoichiometric ratio to form the polyester urethane;
wherein the at least one polyol monomer, the at least one polyacid monomer, the diisocyanate, the first stoichiometric ratio, the second stoichiometric ratio, the degree of branching, the weight average molecular weight, the polydispersity index, and the viscosity are selected to form a degree of phase-separated microstructure between hard segments of the crosslinker and soft segments of the alternating copolymer resin, the degree of phase-separated microstructure providing a predetermined property selected from the group consisting of a degradation rate in an aqueous environment, a release rate of a drug loaded in the polyester urethane in the aqueous environment, a solubility of the drug, and combinations thereof.

14. The process of claim 13 further comprising selecting a drug and selecting a time point of adding the drug selected from the group consisting of adding to the alternating copolymer resin, adding to the flowable blend, and adding to the diisocyanate, wherein the at least one polyol monomer, the at least one polyacid monomer, the diisocyanate, the first stoichiometric ratio, the second stoichiometric ratio, and the time point of adding are selected to form the polyester urethane loaded with the drug having the release rate of the drug in the aqueous environment.

15. The process of claim 13, wherein the at least one polyol monomer comprises glycerol.

16. The process of claim 13, wherein the at least one polyacid monomer is selected from the group consisting of sebacic acid, suberic acid, adipic acid, succinic acid, and combinations thereof.

17. The process of claim 13, wherein the diisocyanate is selected from the group consisting of hexamethylene diisocyanate and lysine diisocyanate.

18. The process of claim 17, wherein the weight average molecular weight of the alternating copolymer resin is less than 6,500 Da.

19. The process of claim 17, wherein the degree of branching is such that the polyester resin has a 1,2,3-triacylglyceride content of less than 15 mol %.

20. The process of claim 17, wherein the degree of branching is such that the polyester resin has a 1,2,3-triacylglyceride content of more than or equal to 15 mol %, and the isocyanate comprises lysine diisocyanate.

21. The process of claim 13, wherein the at least one polyol monomer comprises glycerol, the at least one polyacid comprises sebacic acid, and the isocyanate comprises lysine diisocyanate.

22. The process of claim 21, wherein the at least one polyacid monomer further comprises adipic acid.

23. The process of claim 13 further comprising adding a chain extender selected from the group consisting of 1,4-butanediol, 1,5-pentanediol, 1,6-hexanediol, isopentyldiol, 2-methy1,3-propanediol, bis(2-hydroxyethyl) terephthalate, ethylene glycol, diethylene glycol, propylene glycol, dipropylene glycol, ethylene diamine, 2-hydroxyethyl 2-hydroxypropanoate, and 2,2-bis(hydroxymethyl) propionic acid.

24. The process of claim 23, wherein the chain extender is added in an amount of about 5 to 15 wt % with respect to a weight of the flowable polyester resin.

25. An implantable product comprising a drug and a polyester urethane, wherein the polyester urethane comprises a crosslinker based on a diisocyanate and an alternating copolymer resin of at least one polyol monomer and at least one polyacid monomer, the crosslinker crosslinking the alternating copolymer resin, the alternating copolymer resin having a degree of branching, a weight average molecular weight, a polydispersity index, and a viscosity prior to the crosslinking, the polyester urethane having a first stoichiometric ratio of the at least one polyol to the at least one polyacid, the polyester urethane having a second stoichiometric ratio of isocyanate-to-hydroxyl, the polyester urethane having a degree of phase-separated microstructure between hard segments of the crosslinker and soft segments of the alternating copolymer resin, the crosslinking stabilizing the degree of phase-separated microstructure, the degree of phase-separated microstructure being based on a selected combination of the at least one polyol monomer, the at least one polyacid monomer, the degree of branching, the weight average molecular weight, the polydispersity index, the viscosity, the diisocyanate, the first stoichiometric ratio, and the second stoichiometric ratio, the degree of phase-separated microstructure providing a predetermined property to the composition selected from the group consisting of a degradation rate in an aqueous environment, a release rate of the drug loaded in the polyester urethane in the aqueous environment, a solubility of the drug, and combinations thereof.

26. The implantable product of claim 25, wherein the at least one polyol monomer comprises glycerol.

27. The implantable product of claim 25, wherein the at least one polyacid monomer is selected from the group consisting of sebacic acid, suberic acid, adipic acid, succinic acid, and combinations thereof.

28. The implantable product of claim 25, wherein the diisocyanate is selected from the group consisting of hexamethylene diisocyanate and lysine diisocyanate.

29. The implantable product of claim 28, wherein the weight average molecular weight of the alternating copolymer resin is less than 6,500 Da.

30. The implantable product of claim 28, wherein the degree of branching is such that the polyester resin has a 1,2,3-triacylglyceride content of less than 15 mol %.

31. The implantable product of claim 28, wherein the degree of branching is such that the polyester resin has a 1,2,3-triacylglyceride content of more than or equal to 15 mol %, and the isocyanate comprises lysine diisocyanate.

32. The implantable product of claim 25, wherein the at least one polyol monomer comprises glycerol, the at least one polyacid comprises sebacic acid, and the isocyanate comprises lysine diisocyanate.

33. The implantable product of claim 32, wherein the at least one polyacid monomer further comprises adipic acid.

34. The implantable product of claim 25 further comprising a chain extender selected from the group consisting of 1,4-butanediol, 1,5-pentanediol, 1,6-hexanediol, isopentyldiol, 2-methy1,3-propanediol, bis(2-hydroxyethyl) terephthalate, ethylene glycol, diethylene glycol, propylene glycol, dipropylene glycol, ethylene diamine, 2-hydroxyethyl 2-hydroxypropanoate, and 2,2-bis(hydroxymethyl) propionic acid.

35. The implantable product of claim 34, wherein the chain extender is added in an amount of about 5 to 15 wt % with respect to a weight of the flowable polyester resin.
